(19) United States
(12) Patent Application Publication
(10) Pub. No.: US 2023/0386603 A1
(43) Pub. Date: Nov. 30, 2023

(54) POISSON SIGNAL RECOVERY FROM MULTIPLE MEASUREMENTS

(71) Applicant: William Marsh Rice University, Houston, TX (US)

(72) Inventors: Pavan K. KOTA, Overland Park, KS (US); Richard G. BARANIUK, Houston, TX (US); Daniel E. LEJEUNE, Houston, TX (US); Rebekah A. DREZEK, Spring, TX (US); Hoang-Anh VU, Houston, TX (US)

(73) Assignee: William Marsh Rice University, Houston, TX (US)

(21) Appl. No.: 18/055,793

(22) Filed: Nov. 15, 2022

Related U.S. Application Data
(60) Provisional application No. 63/279,484, filed on Nov. 15, 2021.

(51) Int. Cl. G16B 5/20; G16B 40/10

(52) U.S. Cl. CPC G16B 5/20; G16B 40/10

(57) ABSTRACT

The present disclosure provides methods for quantifying target analytes in sample by providing framework for expanded multiplexing through asynchronous fingerprinting.

### PRIORITY CLAIM

This application claims benefit of priority to U.S. Provisional Application Ser. No. 63/279,484, filed Nov. 15, 2021, the entire contents of which are hereby incorporated by reference.

### STATEMENT REGARDING FEDERALLY SPONSORED RESEARCH

This invention was made with government support under Grant No. T15LM007093 awarded by the National Institutes of Health, Grant No. CBET 2017712 awarded by the National Science Foundation, and Grant No. N00014-18-1-2047 awarded by the Department of Defense and Office of Naval Research. The government has certain rights in the invention.

This application contains a Sequence Listing XML, which has been submitted electronically and is hereby incorporated by reference in its entirety. Said XML Sequence Listing, created on May 7, 2023, is named RICEP0100US.xml and is 7,148 bytes in size.

### BACKGROUND

### 1. Field

The disclosure relates generally to the field of biology and signal processing. More particularly, it concerns methods of compressed sensing methods and systems.

### 2. Related Art

As data increasingly informs critical decision-making, efficient signal acquisition frameworks must keep pace. Modern signals of interest are often high-dimensional but can be efficiently recovered by exploiting their underlying structure through signal processing. The field of compressed sensing (CS), reviewed in (Baraniuk, 2007; Eldar & Kutyniok, 2012), focuses on the recovery of sparse signals from fewer measurements than the signal dimension. Concretely, an N-dimensional signal x* with at most k nonzero entries (in which case x* is said to be k-sparse) can be recovered from a measurement vector y acquired by M<N sensors. The sensors' linear responses to entries of x* define a sensing matrix Φ such that, compactly, y=Φx*. Recovering x* from y is known as the single measurement vector (SMV) problem (Chen et al., 1998; Donoho, 2006; Candes & Tao, 2005; Cohen et al., 2009). In the multiple measurement vector (MMV) problem (Chen & Huo, 2005; Chen & Huo, 2006; Tropp et al., 2006), D measurements are captured in an M×D matrix Y in order to recover X*, an N×D signal matrix. X* is jointly sparse such that only k rows contain at least some nonzero elements. CS has been applied extensively in imaging (Duarte et al., 2008; Willett et al., 2011; Lustig et al., 2008) and communications (Li et al., 2013; Zhu & Giannakis, 2011; Ragheb et al., 2008) and only recently in biosensing (Cleary et al., 2017; Koslicki et al., 2014; Aghazadeh et al., 2016; Ghosh et al., 2020).

Emerging microfluidics technologies in the field of biosensing motivate a new MMV framework. With microfluidics, a single sample can be split into D small-volume partitions such as droplets or nanowells with D on the order of 10³ to 10⁷ (Guo et al., 2012). Microfluidic partitioning captures individual analytes (e.g., cells (Hosokawa et al., 2017; Mazutis et al., 2013); genes (Vogelstein & Kinzler, 1999); proteins (Kim et al., 2012; Yelleswarapu et al., 2019); etc.) in partitions, and analyte quantities across partitions are known to follow a Poisson distribution (Basu, 2017; Moon et al., 2011). The common method to detect a library of analytes with large N is to either dilute samples or split samples into more partitions such that the Poisson distributions reduce to either empty or single-analyte capture, i.e., that columns of X* satisfy ∥x*∥∈{0,1}(Mazutis et al., 2013; Velez et al., 2017). This assumption motivates a straightforward N-class classification problem for each non-empty partition, but it necessitates clear separation between classes even under noise, some prior knowledge of sample concentration, and the generation of many wasteful, empty partitions.

The advent of microfluidics in biosensing has led to portable, cost-effective, and automated assays to be performed on simple chips manufactured with the same platforms that spurred the computing revolution (Luka et al., 2015; Bashir, 2004). However, the core methods of biosensing have largely rested on the paradigm of designing sensors that are each specific to a target analyte. For situations in which many target analytes must be considered, this one-to-one principle scales poorly. Many sensors must be embedded on a single device, samples must be concentrated enough such that a representative subsample can be applied to each sensor, and cross-reactivity of sensors and analytes scales combinatorically (Palka-Santini et al., 2009; MacBeath, 2002). For example, bacterial and fungal infection diagnostics comprise dozens to hundreds of pathogens that may be responsible for a patients' generic symptoms, but samples may exhibit very low concentrations (Sinha et al., 2018; Opota et al., 2015).

Nonspecific or target-agnostic sensing modalities are needed for scalable coverage of many analytes. For example, metagenomic sequencing reads the contents of virtually any sample. Amplicon sequencing is an alternative for microbial diagnostics in both microbiome analysis and infections (Schlaberg, 2020; Wellinghausen et al., 2009). These approaches conduct PCR on ribosomal RNA genes (e.g., 16S for bacteria, 18S or 28S for fungal, among others), that are flanked by conserved regions for priming and exhibit internal sequence differences for taxonomic discrimination (Schlaberg, 2020; Johnson et al., 2019). While sequencing has made strides in clinical practice, its expense and required expertise have hindered its routine use (Schlaberg et al., 2017; Gardy & Loman, 2018), and its low sensitivity is insufficient for some clinical presentations such as bloodstream infections (Moragues-Solanas et al., 2021).

Another category of nonspecific sensing involves “fingerprinting” where a general sensing modality assigns unique signatures to target analytes such that an unknown sample can be read and matched against a database of analytes. Spectroscopic methods are common in this class, profiling wide ranges of proteins, metabolites, and cells. In clinical infections, mass spectrometry has been applied to rRNA amplicons (Ecker et al., 2008) although its use to identify clinical isolates from positive culture is gaining much more traction (Singhal et al., 2015, Ozenci et al., 2018). However, a major limitation of these approaches is the difficulty in analyzing mixtures of analytes (Zhang et al., 2019; Alseekh et al., 2021). For mass spectrometry, this limitation manifests as a need to analyze clinical isolates from polymicrobial samples one at a time.

Microfluidic partitioning technologies offer an avenue for the high throughput, sensitive, and quantitative characterization of heterogenous samples via a fingerprinting approach (Zhang et al., 2019). These systems split an initial sample into thousands or millions of partitions such as droplets or nanowells (Guo et al., 2012). If the analytes are at a limiting dilution, most partitions will be empty with an occasional analyte isolated in its own partition. Formally, analytes are captured according to a Poisson distribution and the Poisson rate parameter λₙ for analyte n dictates the probability of capturing nonnegative integer copies (xₙ) of the analyte such that P(xₙ)=λₙ^{x}^{ₙ}e^{-λ}^{ₙ}/x_{n!} (Basu, 2017; Moon et al., 2011). Among N total analytes indexed by n that are distributed independently among the partitions, single-analyte capture is very likely if Σₙλₙ<0.1 with most partitions remaining empty. This “digital microfluidics” approach guides much of the research in single-cell and single-molecule analysis.

Given the probabilistic isolation of individual analytes, nonempty partitions can be classified one at a time against a database. Researchers have demonstrated classification with high resolution melt (HRM) curve analysis of individual 16S gene amplicons captured in droplets (Velez et al., 2017). Also, surface enhanced Raman spectroscopy (SERS) of isolated bacterial cells (Dina et al., 2017) can assign unique spectra to species, and digital SERS with microfluidic capture has been proposed (Zhang et al., 2019). However, all microfluidic partitioning technologies rest on the assumption that droplets must be individually classified. From a data perspective, N analyte classes must have reliable decision boundaries, making these systems highly sensitive to measurement noise (Langouche et al., 2020). Acquiring enough information from each partition for reliable classification limits the throughput of acquiring partition measurements, and therefore, the volume of sample that can be analyzed (Zhu & Fang, 2013). Moreover, in diagnostics, sample concentrations are rarely known a priori. Samples with higher concentrations can result in multianalyte capture in the same partition, causing errors in classification approaches that assume single-analyte capture. Thus, there is an unmet need for CS methods that can generalize the signal recovery strategy when samples are sparse, a common characteristic of biological samples. For example, samples may contain only a few microbes or mutations of interest among many possibilities (Kota et al., 2022; Quan et al., 2018).

### SUMMARY

In certain embodiments, the present disclosure provides methods for a broad-range sensing method for detecting multiple target analytes in a sample comprising:
(a) assigning fingerprints comprising a unique signature to each of the target analytes with sensors;
(b) splitting the sample into multiple subsamples;
(c) splitting each subsample into multiple partitions;
(d) performing asynchronous fingerprinting by contacting the partitions in each subsample with a subset of the sensors; and
(e) detecting the multiple target analytes through statistical estimation using a reference database of analyte fingerprints.

In particular aspects, the sensors are nonspecific sensors. In some aspects, the method further comprises the use of specific sensors, such as one or two specific sensors in addition to the nonspecific sensors.

In some aspects, statistical estimation comprises defining an objective function based on a modeled distribution and optimizing the objective by adjusting the inferred analyte quantities or concentrations. These approaches could involve maximum likelihood estimation (MLE) or maximum a posteriori estimation (MAP). In some aspects, the quantity of each analyte in each partition is an integer and follows a Poisson distribution with mean given by its true concentration in the sample, wherein the objective function comprises maximum likelihood estimation under the modeled probability distribution and maximum likelihood estimation comprises applying a gradient ascent or descent algorithm. Analyte quantities or concentrations may have a closed form solution, but if not, they can be iteratively solved for by applying a gradient ascent or descent algorithm, such as a SPoRe algorithm comprising:
(a) initializing a value of λ;
(b) computing the gradient based on the modeled probability distribution over a subset of the partition measurements or over the entire set of available measurements, and optionally approximating the gradients with Monte Carlo approximations;
(c) updating λ based on the gradient; and
(d) repeating steps (b)-(c) until convergence of λ.

In some aspects, the partitions are microfluidic partitions.

In some aspects, the method further comprises reading partial or full fingerprints and matching the fingerprints against a database of analytes. In certain aspects, the nonspecific sensors are nucleic acids, primers, probes, antibodies, mass spectra, optical spectra, or phenotypic indicators in various growth conditions. In particular aspects, the nonspecific sensors are nucleotide probes comprising 8-11 nucleotides.

In some aspects, detecting comprises measuring the concentration of said target analytes in said sample. In certain aspects, detecting comprises quantifying the microbial content of the sample.

In certain aspects, the microfluidic partitions comprise droplets, chambers or nanowells. In some aspects, the multiple target analytes in the sample are sparse. In some aspects, the method comprises fewer total sensors than the number of target analytes. For example, in the present studies, five nonspecific probes were used to quantify nine types of bacteria. In certain aspects, the partitions are less than 10 μL in volume. In some aspects, the target analytes are captured in the small volume partitions according to a Poisson distribution with the true Poisson rates of all target analytes totaling to less than 20, i.e. Σₙλₙ*<20.

In some aspects, the target analytes in the sample are sparse. In certain aspects, the sample is split into 50 to 10⁷ partitions.

In certain aspects, the target analytes comprise whole cells, genes, DNA, RNA, proteins, or metabolites. In some aspects, the partitions initially capture whole cells that are subsequently lysed. In certain aspects, the multiple target analytes comprise microbes or mutations of interest.

In some aspects, the microbes comprise bacterial pathogens or fungal pathogens.

In some aspects, the target analytes comprise ribosomal RNA genes selected from 16S, 18S, 23S, or 28S ribosomal RNA genes or other marker regions such as internal transcribed spacer (ITS) and interspace (IS) regions. In certain aspects, the nonspecific probes are nonspecific hydrolysis probes which react with the ribosomal RNA genes and assign binary barcodes to said ribosomal RNA genes. In some aspects, the nonspecific hydrolysis probes comprise 8-11 nucleotides with locked nucleic acid that bind to the ribosomal RNA genes. In some aspects, each ribosomal RNA gene elicits a binary barcode response to the nonspecific hydrolysis probes based on the presence or absence of the probe sequences in the gene.

In certain aspects, the method comprises splitting a sample into microfluidic partitions in a digital PCR (dPCR) system and reading signatures from each partition after performing PCR to detect the presence or absence of target nucleic acids.

In some aspects, the digital PCR readout is the continuously valued, raw fluorescence measurements are used to characterize the intensity of the response of the nucleic acid fragments in the partition to the sensors. In certain aspects, the digital PCR reaction completes and a melt curve is evaluated from each partition to characterize the dissociation of nonspecific probes, including unstructured and structured probe types such as molecular beacons and hairpins, from the PCR products.

In some aspects, the target analytes comprise microbes associated with urinary tract infection, bacterial biofilms in chronic wounds, sepsis, meningitis, or a human microbiome. For example, the bacteria may comprise E. coli, S. aureus, K. pneumoniae, S. pneumoniae, A. baumannii, or Lactobacillus spp. In some aspects, the target analytes are the set of all relevant bacteria and fungi that may appear in a sample for the application such as the relevant organisms in the human gut, vaginal, oral, or other microbiota. In certain aspects, the target analytes include a priority set of bacteria and fungi that exhibit fingerprints that are different from other application-relevant bacteria and fungi, and in which samples containing bacteria and fungi beyond the priority set are flagged as indeterminate.

A further embodiment provides a method for evaluating a solution (e.g., an abundance profile of analytes) in a sample and evaluating a pass/fail criteria for the abundance results comprising:
(a) defining a forward model, probabilistic or deterministic, that maps the analyte content of a partition to the measurement(s) that would be acquired from the partition;
(b) evaluating the expected distribution of measurements based on the forward model and the given abundance profile of analytes;
(c) comparing the expected distribution of measurements against the observed distribution of measurements; and
(d) flagging the solution as “indeterminate” or “failed” based on a tolerance threshold of the difference between the expected and observed distributions.

An additional embodiment provides method for evaluating a candidate solution for the target analyte quantities or concentrations in a sample that has been split into multiple partitions and reporting whether the sample contains an exogenous analyte beyond the set of target analytes comprising:
(a) a modeled probability distribution for the measurements in each partition that are conditional on the analyte quantities the sample and the subset of sensors applied in the partition;
(b) evaluating the expected distribution of measurements based on the modeled probability distribution and the candidate solution;
(c) comparing the expected distribution of measurements against the distribution of observed measurements obtained from the partitions, wherein a sufficient difference in the expected and observed distributions identifies the candidate solution as containing an exogenous analyte.

In some aspects, the comparison of distributions is based on a z square goodness of fit test.

In certain aspects, all individual measurements can be adequately explained by the given panel of potential analytes, but the observed distribution may be sufficiently different from the expected distribution based on the forward model. In some aspects, the distributions are compared using a χ-square goodness of fit test.

It is contemplated that any method or composition described herein can be implemented with respect to any other method or composition described herein. For example, a compound synthesized by one method may be used in the preparation of a final compound according to a different method.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1B: (FIG. 1A) Overview of CS SMV formulation. An N-dimensional signal (x) is acquired with M compressive, linear measurements (y) with M<<N. Given that x is sparse with respect to some library (Φ), algorithms can recover x from the low dimensional measurements. (FIG. 1B) Overview of CS MMV Poisson formulation. The signal and measurements are distributed over D vectors each in X and Y respectively. The signal X is jointly sparse such that most rows are entirely zero, and all values are restricted to nonnegative integers and follow Poisson statistics.

FIGS. 2A-2C: Overview of compressed sensing with microfluidics. (FIG. 2A) Measurements are collected from every droplet containing an integer quantity of biomarkers (e.g., microbial cells or genes). The distribution of the biomarker quantities is Poisson with parameter vector λ which represents the average content per droplet of each biomarker. (FIG. 2B) Graphical representation of splitting an original “bulk” sample into many droplets. (FIG. 2C) Comparison of conventional microfluidics-based diagnostics versus SPoRe.

FIG. 3: Each row of the signal matrix Xₙ follows a Poisson distribution with rate λₙ. In this example, λ₂=2 and A=1.

FIG. 4: (Left) Example heatmap results of true versus recovered bacterial content in samples with 4 random bacteria (k=4). (Right) Recovery performance as a function of k and concentration. SPoRe tolerates multi-bacterial capture in droplets, maintaining the concentration range of standard ddPCR while dramatically increasing the number of targets that can be distinguished (here, N=9).

FIG. 5: The Multiple Measurement Vector (MMV) problem with Poisson signals (MMVP) with one sensor group and noiseless measurements. White squares are zeroes and darker colors represent larger values. Each column x*_{d} of X* is drawn from a Poisson distribution governed by the 2-sparse λ* (i.e., x*_{d}˜Poisson(λ*), i.i.d.).

FIG. 6: Example of MMVP and Sparse Poisson Recovery (SPoRe) with M<k<N: Φ=[1,2,3], λ*=[0.5,0,0.5], and D=1000 measurements under additive white Gaussian noise (AWGN) b˜N(0,σ^{r}) with σ²=0.02. SPoRe attempts to fit the distribution of measurements directly and finds \hat{λ}≈[0.45,0.03,0.44]. For comparison, the [symbol] ₁-Oracle minimizes the measurement error \hat{x}=argmin x∥Y−Φ∥_{F} with X≥0 and Σ_{n,d}x_{n,d}=Σ_{n,d}x*_{n,d} as affine constraints. The estimate λ for the [symbol] ₁-Oracle is then set to the average of the columns of \hat{x}, and in this example, λ≈[0.33,0.31,0.31]. The distributions p(y∥\hat{λ}) for each estimation method are compared against the true distribution p(y|λ*) and the empirical histogram of the D observations.

FIGS. 7A-7C: Performance of SPoRe vs. compressed sensing baseline algorithms over 50 trials. Common settings unless otherwise specified were M=10, N=20, G=1, k=3, D=100, Σζ*=2. (FIG. 7A) Performance as a function of M, with σ² 10⁻⁶ for comparison in an effectively noiseless setting. (FIG. 7B) Performance as a function of AWGN variance with M=10. (FIG. 7C) Performance as a function of Σλ*, with σ²=10⁻² and M=10.

FIG. 8: AWGN tolerance of integer-restricted algorithms over 50 trials with M=2, N=10, G=1, k=3, D=100, Σλ*=2.

FIGS. 9A-9C: SPoRe's performance and behavior as a function of k and Σλ* over 50 trials. Unless otherwise stated, common simulation settings are M=2, N=50, G=1, D=1000, σ²=10⁻². (FIG. 9A) Performance when initialized with standard \hat{λ}=0.1. (FIG. 9B) Performance when initialized with \hat{λ}≈λ*, specifically in =max{ϵ, λ*}. (FIG. 9C) Average variance of partial derivatives for indices n not in supp(λ*) evaluated at \hat{λ}˜λ*.

FIGS. 10A-10B: Performance of SPoRe (solid) vs [symbol] ₁-Oracle GM SMV (dashed) as a function of G. Common settings are k=7, N=50, D=1000, σ²=10⁻². (FIG. 10A) Comparison with Σₙλₙ*=10, motivated by FIG. 9A. (FIG. 10B): Comparison with Σₙλₙ*=1.

FIG. 11: Comparison of average variance of \hat{λ}ₙ from SPoRe versus the ideal Cram'er-Rao (CR) bound as a function of D over 50 trials with n∈supp(λ*), λₙ*=1, M=2, N=50, G=20, σ²=10⁻².

FIG. 12: Summary of ddPCR. A sample is split into many droplets, collected, deposited into a ddPCR plate for amplification, and read after PCR. Endpoint fluorescence measurements indicate the presence or absence of target nucleic acids in droplets. Black rectangles indicate PCR amplicons, and green or blue rectangles correspond with complementary sequences of the HEX and FAM probes, respectively, in the amplicons. Droplets with amplicons without a probe binding site elicit minimal fluorescence, ideally clustering with genuinely empty droplets.

FIG. 13: Accounting for barcode variability across copies of the 16S gene. The white values in C are zero, with the darkest gray representing 1. Each column contains the proportions of the barcodes that each bacterial taxa's 16S gene copies exhibit.

FIGS. 14A-14C: End-to-end example of assay. (FIG. 14A) Nonspecific hydrolysis probes react with the 16S genes of multiple bacteria. Probes assign binary barcodes to 16S genes (non-white colors indicates the probe binds to the bacterial gene). All bacteria except E. cloacae elicited the same barcode across all gene copies, and E. cloacae had an estimated one variant copy. Due to multiplexing and channel limitations of ddPCR, barcodes were captured in subsets of four groups of droplets. (FIG. 14B) Example of raw data from four groups of droplets, each from the same mixed bacterial sample. Raw data is binarized by manual thresholding, overriding most of the effects due to partition specific competition. (FIG. 14C) Sparse Poisson Recovery (SPoRe) algorithm optimizes over all groups simultaneously, accurately reflecting the estimated ground truth (dashed lines).

FIG. 15: Formation of the linear system matrix Z that verifies identifiability for the assay. Non-white squares are 1, and white squares are zero. Each group contributes three rows to Z as described in Example 1, and rank(Z) is N.

FIG. 16: Signal recovery results against the estimated ground truth. All colors are scaled against the maximum estimated ground truth concentration of λ*=2.56 for Concentration 1 of B. fragilis.

FIG. 17: Likelihood comparison of SPoRe's solution against the estimated ground truth. SPoRe's solution exhibits higher average likelihood for the pre-binarized data that it is given.

FIG. 18: SPoRe's performance on random subsamples of experimental data. Each sample's population of prebinarized droplet measurements was subsampled by a factor of 10⁻¹, 10⁻²,10⁻³, and 10⁻⁴. SPoRe performed MLE on the subsampled data.

FIG. 19: Flagging samples with out-of-panel bacteria. A χ-squared goodness of fit test is performed using the distribution of y expected given \hat{λ} and the observed distribution. The p value of the test is used as the metric for determining if a sample has an unknown barcode (e.g., SPoRe was not given one of the present barcodes in the solution).

FIG. 20: Plate layout for ddPCR test samples. The first two rows served as references for ground truth concentration estimation of monomicrobial dilutions and manual thresholding of all wells. Random mixtures of bacteria were distributed across the rest of the plate with each mixture being applied to four wells, each with a different subset of two probes defining the 16S barcodes.

FIGS. 21A-21C: Example process for manual thresholding with noted challenges. (FIG. 21A) All reference data coming from the same probe group was pooled and displayed to serve as a visual reference. Droplet “rain” is evident in each cluster. (FIG. 21B) Raw data from a polymicrobial sample was overlaid on the reference data with the same corresponding probe group. An example is shown with the raw data from D09 (Group 1, k=4 sample number 2) overlaid with the Group 1 reference data. PSC effects, as expected, create subpopulations of droplet measurements between the binary clusters. The small additional cluster near zero may be due to droplets where probes partially interacted with amplicons due to imperfect sequence homology. (FIG. 21C) After the user selects two points to define a line for thresholding, the plot is updated to allow the user to visually confirm the results. Red points are assigned the value 1, and black points are assigned 0.

FIG. 22: Separation of bacterial barcodes with amplitude multiplexing. Each cluster here is from a separate ddPCR reaction with one bacterial species in it. Data from the three Staphylococcus bacterial species and the two Streptococcus species were combined in this plot.

FIG. 23: Example of partial barcode matrix for Group 1.

FIG. 24: SPoRe's Performance on Simulations of Experimental Concentrations. Given the estimated ground truth concentrations (λ*), simulated binary measurement data was used to pass to SPoRe. SPoRe returns virtually perfect results with mean cosine similarity of 0.9997.

FIGS. 25A-25E: (FIG. 25A) Schematic depicting lysis of whole cells within microfluidic partitions and reading out of “keyword” frequencies—measurements based on the frequency of short subsequences of DNA in the cell's genome. (FIG. 25B) Approximate limit of detection for completed demonstration with ddPCR. For each sample, took a random subsample of real data s E {10⁻¹, 10⁻², 10⁻³, 10⁻⁴} was taken and used to estimate copy count in each sample sD (Σₙλₙ*). (FIG. 25C) Simulation of comprehensive coverage with non-sparse samples and 150 panel size (k=N=150). G=number of subsamples or groups (e.g., 4 groups in demonstration in slide above). Droplet digital PCR plots assume binary measurements, whereas the green curve assumes a linear measurement model such that could be achieved with “keyword” measurements. Maximum panel size assumes one probe per channel collecting one binary measurement; resolving more than one probe per channel is established with amplitude multiplexing. (FIG. 25D) Probe binding information with select bacteria. 1 indicates probe has exact sequence match in bacterial 16S gene and 0 indicates no match. The probes are designed to account for more than 100 types of bacteria, such as the 6 bacteria depicted. (FIG. 25E) ddPCR data. Some bacteria can react “partially” to different probes. E. faecium has 0/0 response to probes 15, 16, but appears to have a slight HEX response (probe 15). Likewise, E cloacae should only react with probe 15 but has a y-axis (FAM) response to probe 16 that is weaker than K. pneumoniae's. These “partial” hits are generally only a source of more information. Rather than just measuring each droplet to be “positive” or “negative” in each color channel, these fractional responses give more information to work with. The particular positions can be calibrated and passed to SPoRe rather than binarizing the data first.

FIGS. 26A-26D: (FIG. 26A) Exemplary probe design. Probe on left is a universal probe set that distinguishes all bacteria. Probe on left focuses on a priority panel. The bacteria in the black box can overlap with each other or with the panel. The algorithm does not penalize or award pairwise distinctions of bacteria in the black box. (FIG. 26B) Targeted panel probe design with 14 different probes of 8-9 nucleotides. Barcode signature aligns with intended panel, such as among species of Stapylococcus, all of their barcode signatures only overlap with other Staphylococcus bacteria. (FIG. 26C) Designed for a priority panel, but the selected probes still assign unique barcode signatures to many of the other relevant bacteria. Accuracy of barcode signatures based on available data from RefSeq (NIH public database) at time of analysis. (FIG. 26D) Probe length and scaling. Generally, specifying a “panel” of priority or intended microbes to detect simplifies the design problem since fewer probes can be used to distinguish this subset of microbes from all others (as illustrated in FIG. 26B). Shorter probes appear to scale more efficiently in distinguishing microbes, at least among the comparison of 8-mer against 10 and 11-mer probes, which could be because short probes are less specific and more readily access barcodes with more positive hits.

### DESCRIPTION OF ILLUSTRATIVE EMBODIMENTS

Current diagnostic methods use specific sensors (e.g., primers/probes, antibodies, etc.) for detecting analytes (e.g., whole cells, target DNA/RNA, protein, etc.). These methods are specific and are designed to sense one analyte. Broad-range sensing can use a mechanism to detect multiple analytes, such as sample culture, sequencing, spectrometry, and melt curve analysis. There is an unmet need to extract more information from simple sensors for a rapid, efficient method for broad-range sensing.

A few nonspecific sensors can efficiently assign fingerprints to many analytes and compressed sensing can unmix fingerprints to quantify analytes if samples are sparse. Diagnostics with microfluidic partitioning means analytes are captured according to a Poisson distribution. At times, the number of sensors required for distinguishing analyte fingerprints exceeds what can be resolved from single individual reactions. Thus, in certain embodiments, the present methods provide framework for expanded multiplexing through asynchronous fingerprinting.

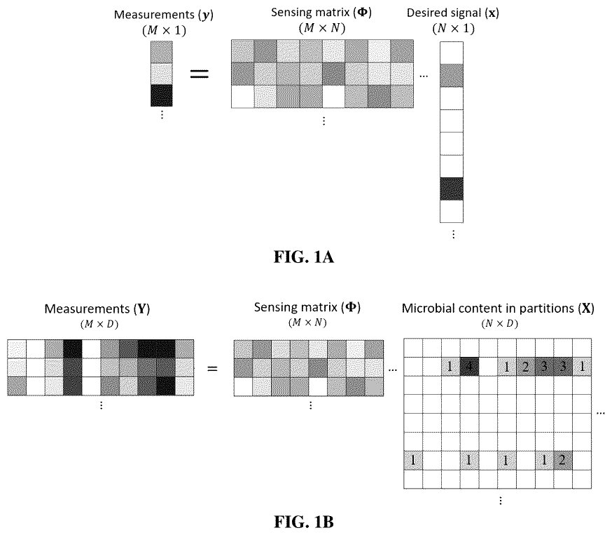

Compressed sensing (CS) problems typically follow the single-measurement vector (SMV) formulation (FIG. 1). The vector x represents an N-dimensional sparse signal that is sought to be recovered with M linear measurements where M is far fewer than N. The sensing matrix Φ represents the core components or atoms that may make up the underlying signal; y represents a set of M measurements that is a linear combination of these atoms. This formulation reduces to a simple representation with linear algebra: y=Φx. In previous work (Aghazadeh et al., 2016), CS has been applied for microbial diagnostics such that M probes were used to quantify N microbes and Φ's columns stored microbial fingerprint responses to the probes.

Given Φ and having measured y, CS solves for x by accounting for assumptions on its structure. Most commonly, x is assumed to be sparse (most of its elements are zero) such that sparse recovery algorithms can recover x from y and Φ.

In the Multiple Measurement Vector (MMV) framework, a set of signals with the same support must be recovered from their corresponding measurements. The present studies demonstrate the first exploration of the MMV problem where signals are independently drawn from a sparse, multivariate Poisson distribution. The method may be applied to a suite of biosensing applications of microfluidics where analytes (such as whole cells or biomarkers) are captured in small volume partitions according to a Poisson distribution. The sparse parameter vector of Poisson rates is recovered through maximum likelihood estimation with the present novel Sparse Poisson Recovery (SPoRe) algorithm. SPoRe uses batch stochastic gradient ascent enabled by Monte Carlo approximations of otherwise intractable gradients. By uniquely leveraging the Poisson structure, SPoRe substantially outperforms a comprehensive set of existing and custom baseline CS algorithms. Notably, SPoRe can exhibit high performance even with one-dimensional measurements and high noise levels. This resource efficiency is not only unprecedented in the field of CS but is also particularly potent for applications in microfluidics in which the number of resolvable measurements per partition is often severely limited. The present studies showed the identifiability property of the Poisson model under such lax conditions, analytically developed insights into system performance, and confirmed these insights in simulated experiments. The present methods can be applied to biosensing and are generalizable to other applications featuring spatial and temporal Poisson signals.

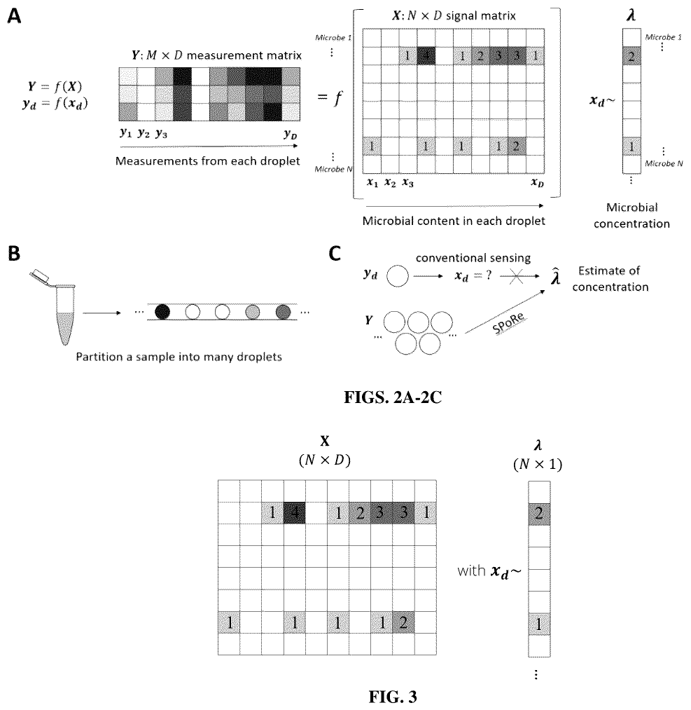

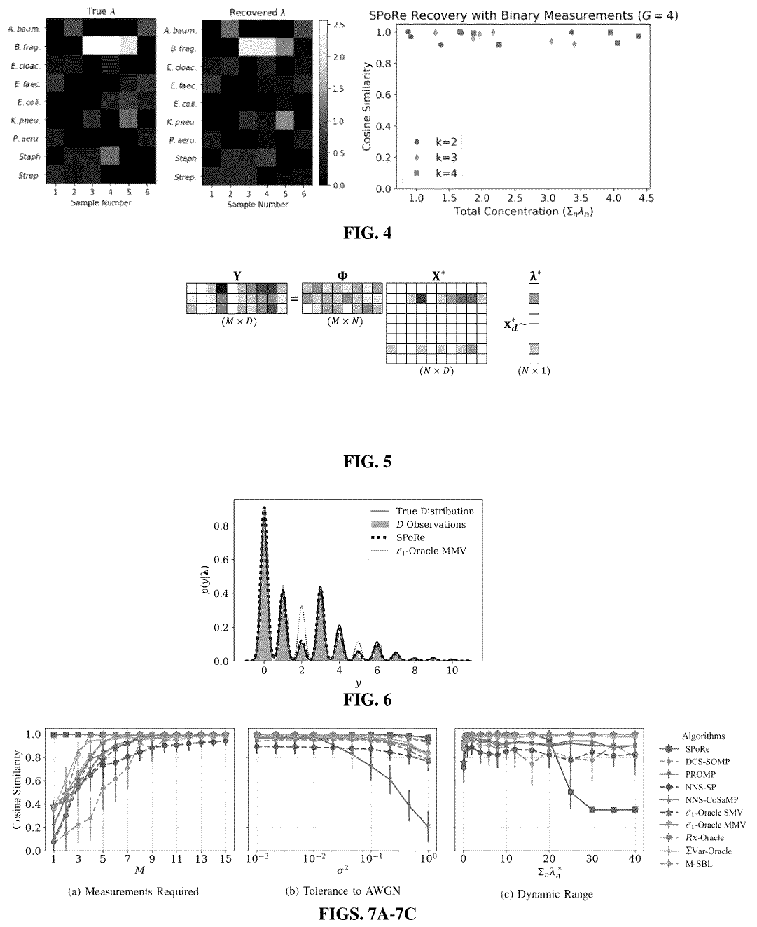

Thus, in certain embodiments, the present methods comprise the use of a recovery algorithm that applies additional prior knowledge beyond sparsity for specialized scenarios, depicted in FIG. 2. Instead of focusing on one measurement and one desired vector, multiple measurement vectors (MMV) and multiple signals are considered. The measurements and signals are stored in the matrices Y and X, respectively, and the measurement model for a particular group is generalized to ƒ such that Y=ƒ(X) with ƒ(X)=ΦX in the special case of a linear model. X is jointly sparse, meaning that entire rows are zero with only a few rows containing some nonzero elements. n is defined as a row index ranging from 1 to N, d as a column index ranging from 1 to D.

MMV problems are well studied, but it is a special case when these nonzero elements in X also follow a Poisson distribution in each row. Each row Xₙ is described by its own Poisson parameter λₙ. At its simplest, this parameter represents the average value in any given row, but it also parametrizes the probability that a particular element X_{n,d} takes on the integer value x:

$$P ( {X}_{n , d} = x \vert {λ}_{n} ) = \frac{{λ}_{n}^{x} {e}^{- {λ}_{n}}}{x !}$$

It can be assumed that each signal vector (column of X) is independently and identically distributed; this assumption makes the probability independent of the particular signal index d.

The present algorithm does not recover X but rather finds the Poisson rate vector λ that characterizes X (FIG. 3). Because λ is equivalent to the average value in each row, recovering λ captures the relative contribution of atoms to the measurements. In envisioned applications, these abundances are more important than the exact values that populate X.

To find λ, a maximum likelihood estimation (MLE) problem was set up; the λ was sought that maximizes the likelihood of the measurements (Y) given Φ and Poisson constraints. an estimate of λ can be iteratively adjusted with a common optimization technique known as stochastic gradient descent by processing batches of measurement vectors from Y. The present studies describe the mathematical details, the theory, and simulated results. The simulations indicated very high noise robustness and far better measurement efficiency (i.e., fewer required measurements, M) than conventional SMV CS. Another finding in the theory is that while signal sparsity is helpful in achieving stable recovery, it is not necessary for estimating λ, broadening the viable application of the present embodiments.

### I. DEFINITIONS

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

The term “essentially” is to be understood that methods or compositions include only the specified steps or materials and those that do not materially affect the basic and novel characteristics of those methods and compositions.

The terms “substantially” or “approximately” as used herein may be applied to modify any quantitative comparison, value, measurement, or other representation that could permissibly vary without resulting in a change in the basic function to which it is related.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

The term “about” means, in general, within a standard deviation of the stated value as determined using a standard analytical technique for measuring the stated value. The terms can also be used by referring to plus or minus 5% of the stated value.

The phrase “effective amount” or “therapeutically effective” means a dosage of a drug or agent sufficient to produce a desired result. The desired result can be subjective or objective improvement in the recipient of the dosage, increased lung growth, increased lung repair, reduced tissue edema, increased DNA repair, decreased apoptosis, a decrease in tumor size, a decrease in the rate of growth of cancer cells, a decrease in metastasis, or any combination of the above.

“Subject” and “patient” refer to either a human or non-human, such as primates, mammals, and vertebrates. In particular embodiments, the subject is a human.

As used herein, the terms “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), diminishment of extent of the deficit, stabilized (i.e., not worsening) state of a tumor or malignancy, delay or slowing of tumor growth and/or metastasis, and an increased lifespan as compared to that expected in the absence of treatment.

A “nucleic acid molecule” or “nucleic acid sequence” refers to any single-stranded or double-stranded nucleic acid molecule including standard canonical bases, hypermodified bases, non-natural bases, or any combination of the bases thereof. For example and without limitation, the nucleic acid molecule contains the four canonical DNA bases—adenine, cytosine, guanine, and thymine, and/or the four canonical RNA bases—adenine, cytosine, guanine, and uracil. Uracil can be substituted for thymine when the nucleoside contains a 2′-deoxyribose group. The nucleic acid molecule can be transformed from RNA into DNA and from DNA into RNA. For example, and without limitation, mRNA can be created into complementary DNA (cDNA) using reverse transcriptase and DNA can be created into RNA using RNA polymerase. “Analogous” forms of purines and pyrimidines are well known in the art, and include, but are not limited to aziridinylcytosine, N⁴-acetylcytosine, 5-fluorouracil, 5-bromouracil, 5-carboxymethylaminomethyl-2-thiouracil, 5-carboxymethylaminomethyluracil, inosine, N⁶-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N⁶-methyladenine, 7-methylguanine, 5-methylaminomethyluracil, 5-methoxyaminomethyl-2-thiouracil, beta-d-mannosylqueosine, 5-methoxyuracil, 2-methylthio-N⁶-isopentenyladenine, uracil-5-oxyacetic acid methyl ester, pseudouracil, queuosine, 2-thiocytosine, 5-methyl-2-thiouracil, 2-thiouracil, 4-thiouracil, 5-methyluracil, uracil-5-oxyacetic acid, and 2,6-diaminopurine. The nucleic acid molecule can also contain one or more hypermodified bases, for example and without limitation, 5-hydroxymethyluracil, 5-hydroxyuracil, α-putrescinylthymine, 5-hydroxymethylcytosine, 5-hydroxycytosine, 5-methylcytosine, 5-methylcytosine, 2-aminoadenine, α-carbamoylmethyladenine, N⁶-methyladenine, inosine, xanthine, hypoxanthine, 2,6-diaminopurine, and N⁷-methylguanine. The nucleic acid molecule can also contain one or more non-natural bases, for example and without limitation, 7-deaza-7-hydroxymethyladenine, 7-deaza-7-hydroxymethylguanine, isocytosine (isoC), 5-methylisocytosine, and isoguanine (isoG).

The nucleic acid molecule containing only canonical, hypermodified, non-natural bases, or any combinations the bases thereof, can also contain, for example and without limitation where each linkage between nucleotide residues can consist of a standard phosphodiester linkage, and in addition, may contain one or more modified linkages, for example and without limitation, substitution of the non-bridging oxygen atom with a nitrogen atom (i.e., a phosphoramidate linkage, a sulfur atom (i.e., a phosphorothioate linkage), or an alkyl or aryl group (i.e., alkyl or aryl phosphonates), substitution of the bridging oxygen atom with a sulfur atom (i.e., phosphorothiolate), substitution of the phosphodiester bond with a peptide bond (i.e., peptide nucleic acid or PNA), or formation of one or more additional covalent bonds (i.e., locked nucleic acid or LNA), which has an additional bond between the 2′-oxygen and the 4′-carbon of the ribose sugar. The term “2′-deoxyribonucleic acid molecule” means the same as the term “nucleic acid molecule” with the limitation that the 2′-carbon atom of the 2′-deoxyribose group contains at least one hydrogen atom. The term “ribonucleic acid molecule” means the same as the term “nucleic acid molecule” with the limitation that the 2′-carbon atom of the ribose group contains at least one hydroxyl group.

“Contacting” means a process whereby a substance is introduced by any manner to promote an interaction with another substance.

“Detecting a nucleic acid molecule” means using an analytical method that can determine the presence and/or quantity of the nucleic acid of interest or that can determine more detailed information regarding the nucleic acid sequence, alterations of a nucleic acid sequence when compared with a reference sequence, or the presence or absence of one or more copies of the nucleic acid sequence.

The term “label” or “probe sequence” as used herein means a molecule or moiety having a property or characteristic which is capable of detection. A label or probe sequence can be directly detectable, as with, for example, radioisotopes, fluorophores, chemiluminophores, enzymes, colloidal particles, and fluorescent microparticles; or a label may be indirectly detectable, as with, for example, specific binding members. It will be understood that directly detectable labels may require additional components such as, for example, substrates, triggering reagents, and light to enable detection of the label. When indirectly detectable labels are used, they are typically used in combination with a “conjugate.” A conjugate is typically a specific binding member which has been attached or coupled to a directly detectable label. Coupling chemistries for synthesizing a conjugate are well known in the art and can include, for example, any chemical means and/or physical means that does not destroy the specific binding property of the specific binding member or the detectable property of the label.

“Hybridize” or “hybridization” refers to the binding between nucleic acids. The conditions for hybridization can be varied according to the sequence homology of the nucleic acids to be bound. Thus, if the sequence homology between the subject nucleic acids is high, stringent conditions are used. If the sequence homology is low, mild conditions are used. When the hybridization conditions are stringent, the hybridization specificity increases, and this increase of the hybridization specificity leads to a decrease in the yield of non-specific hybridization products.

However, under mild hybridization conditions, the hybridization specificity decreases, and this decrease in the hybridization specificity leads to an increase in the yield of non-specific hybridization products.

A “nucleotide probe” or “probes” refers to a polynucleotide that is at least eight (8) nucleotides in length and which forms a hybrid structure with a target sequence, due to complementarity of at least one sequence in the probe with a sequence in the target region. The polynucleotide can be composed of DNA and/or RNA or any of the analogous or modified bases described above. Probes in certain embodiments, are detectably labeled. Probes can vary significantly in size. Generally, probes are, for example, at least 8 to 15 nucleotides in length. Other probes are, for example, at least 20, 30 or 40 nucleotides long. Still other probes are somewhat longer, being at least, for example, 50, 60, 70, 80, or 90 nucleotides long. Probes can be of any specific length that falls within the foregoing ranges as well. Preferably, the probe does not contain a sequence complementary to the sequence(s) used to prime for a target sequence during the polymerase chain reaction.

“Oligonucleotide” or “polynucleotide” refers to a polymer of a single-stranded or double-stranded deoxyribonucleotide or ribonucleotide, which may be unmodified RNA or DNA or modified RNA or DNA.

A “modified ribonucleotide” or deoxyribonucleotide refer to molecules that can be used in place of naturally occurring bases in nucleic acid and includes, but is not limited to, modified purines and pyrimidines, minor bases, convertible nucleosides, structural analogs of purines and pyrimidines, labeled, derivatized and modified nucleosides and nucleotides, conjugated nucleosides and nucleotides, sequence modifiers, terminus modifiers, spacer modifiers, and nucleotides with backbone modifications, including, but not limited to, ribose-modified nucleotides, phosphoramidates, phosphorothioates, phosphonamidites, methyl phosphonates, methyl phosphoramidites, methyl phosphonamidites, 5′-p-cyanoethyl phosphoramidites, methylenephosphonates, phosphorodithioates, peptide nucleic acids, achiral and neutral internucleotidic linkages.

A “primer” or “primer sequence” refers to an oligonucleotide that hybridizes to a target nucleic acid sequence (for example, a DNA template to be amplified) to prime a nucleic acid synthesis reaction. The primer may be a DNA oligonucleotide, an RNA oligonucleotide, or a chimeric sequence. The primer may contain natural, synthetic, or modified nucleotides. Both the upper and lower limits of the length of the primer are empirically determined. The lower limit on primer length is the minimum length that is required to form a stable duplex upon hybridization with the target nucleic acid under nucleic acid amplification reaction conditions. Very short primers (usually less than 3-4 nucleotides long) do not form thermodynamically stable duplexes with target nucleic acid under such hybridization conditions. The upper limit is often determined by the possibility of having a duplex formation in a region other than the pre-determined nucleic acid sequence in the target nucleic acid. Generally, suitable primer lengths are in the range of about 10 to about 40 nucleotides long. In certain embodiments, for example, a primer can be 10-40, 15-30, or 10-20 nucleotides long. A primer is capable of acting as a point of initiation of synthesis on a polynucleotide sequence when placed under appropriate conditions.

“Detection,” “detectable” and grammatical equivalents thereof refer to ways of determining the presence and/or quantity and/or concentration and/or identity of an analyte. In some embodiments, detection occurs during or after the amplification of the target nucleic acid sequence. In other embodiments, sequencing of the target nucleic acid can be characterized as “detecting” the target nucleic acid. A label attached to the probe can include any of a variety of different labels known in the art that can be detected by, for example, chemical or physical means.

Labels that can be attached to probes may include, for example, fluorescent and luminescence materials.

“Amplifying,” “amplification,” and grammatical equivalents thereof refers to any method by which at least a part of a target nucleic acid sequence is reproduced in a template-dependent manner, including without limitation, a broad range of techniques for amplifying nucleic acid sequences, either linearly or exponentially. Exemplary means for performing an amplifying step include ligase chain reaction (LCR), ligase detection reaction (LDR), ligation followed by Q-replicase amplification, PCR, primer extension, strand displacement amplification (SDA), hybridization chain reaction (HCR), hyperbranched strand displacement amplification, multiple displacement amplification (MDA), nucleic acid strand-based amplification (NASBA), two-step multiplexed amplifications, rolling circle amplification (RCA), recombinase-polymerase amplification (RPA) (TwistDx, Cambridg, UK), and self-sustained sequence replication (3SR), including multiplex versions or combinations thereof, for example but not limited to, OLA/PCR, PCR/OLA, LDR/PCR, PCR/PCR/LDR, PCR/LDR, LCR/PCR, PCR/LCR (also known as combined chain reaction-CCR), and the like. Descriptions of such techniques can be found in, among other places, Sambrook et al. Molecular Cloning, 3^{rd} Edition).

The term “analyte” refers to the unit intending to be detected. In some embodiments, it includes nucleic acids such as a gene, gene region, or whole genome. In other embodiments, an analyte may include a polypeptide, metabolite, unique cell state, or an organism or group of organisms belonging to a particular taxonomic grouping.

The term “signal” refers to the true analyte quantities or concentrations in a single reaction (e.g., a microfluidic partition) or the whole sample that is inferred based on the acquired measurements. In our mathematical notation, the signal consists of N analyte quantities, concentrations, or Poisson parameters.

The term “sparse” refers to signals with relatively few nonzero elements. Particularly, an N dimensional signal that characterizes the quantities or concentrations of N analytes is said to be k-sparse if N-k or more of its elements are zero or approximately zero. In some embodiments, many (N) analytes must be accounted for in a sample, but it can be assumed that any given sample only has a small subset (k or fewer) of the possible analytes. In these cases, the sparse signal is the analyte concentrations or quantities.

The term “measurement” refers to a unit of information directly acquired from each reaction (e.g., a microfluidic partition) represented by a scalar value.

The term “sensing channel” refers to a system component that translates the output of sensors into a measurement. In some embodiments, it is an optical wavelength band (e.g., from fluorescence, luminescence, reflectance, etc.) that captures the intensity of acquired light within that band. In similar embodiments, it may refer to any unit or range of physical properties (e.g., mass, mass-to-charge, temperature, etc.) that captures the absolute or relative abundance of analytes in the reaction at that unit or within the range. It may also be agnostic to a particular band of information, capturing the total incident light, mass, charge, voltage, current, or other physical or electrical unit. In other embodiments, a sensing channel is defined by its physical location; for instance, a sensor may be immobilized on an array such that the location is linked to the sensor's identity, and the resulting measurement corresponds with that location. In other embodiments, a sensing channel is defined by the unit of time at which the measurement is captured. In still other embodiments, it may be the result of representing a component of an arbitrarily long nucleic acid sequence read from a sequencing instrument. For example, the sensing channel may report the quantity of a certain subsequence among one or more sequencing reads (Koslicki et al., 2014).

The term “sensor” refers to that which captures information from a sample based on the analyte content and transduces it to a sensing channel. In many embodiments, the sensor and sensing channel are directly linked; that which captures the information, e.g., a photodetector capturing light is directly connected to a circuit that digitizes the value to translate the photons into a measurement. In optical spectrometry, a sensor reports the intensity of light corresponding with a wavelength or band of wavelengths. In some embodiments, a sensor could be a biosensor (nucleic acid probes, antibodies, etc.) that is optically labeled (e.g., with a fluorophore) such that its interactions with analytes result in a change in the light emitted within a certain optical wavelength band (the sensing channel). In some embodiments involving composite measurements (Cleary et al., 2017), a sensor could be a set of multiple biosensors that each target one or more separate analytes but are tagged with the same label such that all biosensors in the set report to the same sensing channel. The resulting measurement is the aggregate response of all sensors. The term “nonspecific sensor” refers to a sensor that is intentionally designed to report a nonzero measurement value through a sensing channel in response to more than one particular analyte such that a nonzero measurement does not indicate the presence or quantity of any single analyte with high probability. It could include nucleotide probes that bind to more than one type of genetic biomarker or organism, units of a spectral measurement system (wavelength, mass, temperature, etc.) that are nonunique to any single molecule or organism, or nonunique nucleotide sequences acquired by a sequencing instrument. It could also be a composite measurement as discussed above.

In other embodiments, it may be a cell growth or viability indicator for a cell or population of cells in a particular environment.

The term “fingerprint” of an analyte refers to the unique and expected measurements from a set of sensors measuring one copy, isolate, molecule, or unit of the analyte. By unique, it is meant that all analytes under consideration exhibit a distinct fingerprint. In some embodiments, a fingerprint is a single vector representing the expected measurements containing the same number of elements as the number of sensors. In some embodiments, the analyte has multiple states such that the fingerprint is a mixture distribution of vectors of expected measurements. For instance, in FIG. 13, the fingerprint for each taxonomical group of bacteria (the analyte) is a barcode distribution that accounts for sequence variants of a bacterial 16S gene in a single genome. In some embodiments, a fingerprint is the unique spectral response of a compound or organism (e.g., acquired by mass spectrometry). In other embodiments, it is the binding pattern of a set of nucleotide probes to a genetic biomarker (the analyte), including genes, genetic regions, or whole genomes. The fingerprint can be defined by the expected number of hybridization events between the probes and biomarkers or by calibrated measurements such as the expected fluorescence response with labeled nucleotide probes. In other embodiments, it is a signature melt curve when a double-stranded nucleotide sequence's dissociation is quantified with changing temperature, eliciting a measurement for each increment of temperature. In other embodiments, it is a signature temporal response of an analyte under a particular environmental condition, or the combined temporal responses of an analyte under multiple environmental conditions.

The term “small volume” refers to relatively small fractions of an initial sample volume.

In some embodiments, an initial sample is split via microfulidics into hundreds of partitions or more such that each “small volume” partition is 1% or less of the initial sample volume. Although it could depend on the specific system in question, partition volumes are generally 10 μL or less.

In the specific studies below with digital PCR, partition volumes were 1 nL with initial sample volumes of 20 μL.

The term “fingerprinting” refers to a method for detecting analytes in which a set of sensors assigns unique fingerprints to all analytes under consideration, and the fingerprints are stored in a reference database.

The term “asynchronous fingerprinting” refers to a type of fingerprinting method for detecting analytes in which all of the sensors required for uniquely assigning fingerprints to the analytes under consideration are not applied simultaneously in any given reaction volume. Instead, a sample is split into multiple subsamples (or “groups”) with subsets of the sensors applied in each group. Asynchronous fingerprinting may generally be used when the number of sensing channels available for a given reaction volume is outnumbered by the number of sensors necessary for fingerprinting. For instance, in a microfluidic partition with a single copy of an analyte, the entire analyte fingerprint is not measured at once for the single copy. In some embodiments, this involves P nonspecific probes that assign fingerprints to genetic biomarkers, where the system acquires data from fewer than P (e.g., M) probes at a time. In the in vitro studies below, P=5 probes assign fingerprints to nine bacterial taxonomic groups, but subsets of M=2 probes are measured in four subsamples, wherein each subsample is split into thousands of microfluidic partitions (droplets).

In other embodiments where the fingerprint is spectrally based, asynchronous fingerprinting refers to capturing portions of the spectra in each group. In another embodiment, the fingerprint may be a signature response of a cell type to multiple environments. Each group could feature a different set of reaction conditions (e.g., growth media) such that a cell type (e.g., microbial species) has a fingerprint combination of responses to all tested environments, but only one environment can be applied at a time in any given group.

The term “concentration” of an analyte is used as a general term for the number of analyte copies in a unit of volume, a standard definition to one skilled in the art. As a special case use of the term, when Poisson statistics are assumed such as in the distribution of capture of analyte copies across microfluidic partitions, the Poisson rate parameter (λ) of an analyte is often used interchangeably with the analyte's “concentration.”

### II. SPARSE POISSON SIGNAL RECOVERY

In certain embodiments, the present methods comprise use of an algorithm that recovers sparse, high dimensional Poisson signals from low dimensional measurements. Further embodiments provide use of the algorithm for biomedical application. Biomedical technologies are increasingly applying “digital microfluidics” to capture analytes (e.g., cells) in thousands of individual chambers for analysis. The count of analytes in each chamber follows a Poisson distribution. Analytes are considered to be sparse if there are only a few unique analytes (e.g., cell types) in a given sample relative to the library of possible analytes. Rather than quantifying each analyte with its own specific sensor, a few sensors may be utilized that broadly react with the library of analytes to give each analyte a fingerprint response represented by a low dimensional vector. Low-dimensional measurements are collected from all chambers and account for Poisson statistics to quantify the concentration of the few unique analytes in a given sparse sample.

Emerging biomedical applications of “digital microfluidics” (DM) technologies enable researchers and clinicians to capture individual analytes (e.g., cells) from patient or environmental samples in thousands of chambers, and the rate of cell capture follows a Poisson distribution. The present algorithm may be used for diagnostic and prognostic applications where relatively few unique cell types are present (i.e., samples are sparse). For example, the present algorithm may be applied to clinical infection diagnostics where only one or a few unique pathogens are responsible for any given patient's condition.

DM is well known to be capable of single-cell resolution, an advantage in infection diagnostics where samples may contain less than ten copies (colony forming units, CFU) of a target pathogen (Table 1). Moreover, background contamination such as a human cell is naturally isolated in only one sample compartment. Any interference from contamination corrupts a single measurement among thousands in the MMV DM setup but can easily invalidate entire SMV “test tube” assays.

TABLE 1
Examples of clinical infections that present with
many possible pathogens and broad dynamic ranges.

| Sample type | Dynamic Range (CFU/mL) |
|---|---|
| Sepsis | 1-10⁵ |
| Pneumonia | 10³-10⁶ |
| Urinary tract infections | 10⁴-10⁶ |
| Soft tissue infections | 10⁵-10⁹ |

Despite these advantages, DM's utility in infection diagnostics has two limiting assumptions. First, like most rapid diagnostic assays, DM uses highly specific sensors to sense for the presence or absence of target analytes in each chamber which severely limits the scope of assays. DM platforms can generally only screen for only a handful of analytes (e.g., genes, cell types) for each sample because of these sensor limitations, while infections can be caused by any of dozens to hundreds of pathogens. A framework that leverages CS enables exponential measurement efficiency for broad pathogen coverage. The present methods introduce Poisson statistical constraints to CS such that even a single sensor can distinguish between cell types.

Second, samples in DM are assumed to be dilute enough for single-cell capture (Σₙλₙ*≤0.1). More concentrated samples result in frequent multi-cell capture which makes downstream quantification difficult. The present algorithm can handle much higher concentrations. Achieving single-cell characterization while tolerating higher loading capacities allows for infection diagnostics when the pathogen concentration in a patient sample is unknown. Table 1 shows that many relevant clinical scenarios present with vastly different concentrations. The present algorithm enables at least two orders of improvement in dynamic range—the range of concentrations that can be covered in a single assay.

Nucleic acid (DNA or RNA) sequencing is the only competing technology which offers better breadth of coverage. Virtually any organism can be identified with a single technology coupled with ever growing public databases. However, sequencing still suffers from cost and time-limitations that limit its routine use in diagnostics. Importantly, run time and cost scales with sample concentration such that low concentrations result in very long time to results, hindering time sensitive diagnosis of infections to inform treatment selection.

In some embodiments, the present algorithm may be used for detecting ribosomal RNA, such as 16S rRNA. 16S rRNA gene is shared by almost all bacteria but has internal sequence differences based on the bacterial genus or species. Usually, DNA sequencing of 16S genes is used to read them and match them to databases. The present methods can be used to skip the need to do full DNA sequencing by probing particular “keywords” or “barcodes” of the gene (e.g., 8-11 nucleotides). These keywords assign barcode signatures to different 16S gene based on the presence or absence of the keyword in the gene. Bacteria have multiple 16S copies that are largely identical, but occasionally have small differences. Thus, in some aspects, each bacteria does not get just one barcode, but a distribution of barcodes (i.e., barcode signature) that are elicited by the probes. 5 copies, 1 of which is a variant, leads to 80% of gene copies with one barcode and the variant (20% of copies) has a different barcode. The present studies use short (e.g., 8-11 nucleotides) hydrolysis probes spiked with locked nucleic acids. The probes may alternatively comprise a molecular beacon (Chakravorty et al, Rapid universal identification of bacterial pathogens from clinical cultures by using a novel sloppy molecular beacon melting temperature signature technique, J. Clin. Microbiol., 2010).

In some aspects, each target analyte (e.g., bacteria) is not given a barcode, but the cluster positions of each bacteria is calibrated in the fluorescence channels (e.g., a multivariate Gaussian fit). This method can be used to account for partial, non-binary hits and allow the capture of more information with a few measurement channels, rather than hard thresholding and binarizing the data. For example, each bacteria has multiple 16S copies and can be modeled as a mixture distribution based on the responses of individual copies.

A. Scalable Microbial Sensing with Microfluidic Partitioning

In certain embodiments, the present methods provide a new framework for microbial diagnostics that enables the quantification of large panels of microbes with fewer sensors than ever before. This resource efficiency could enable routine infection diagnostics at the point-of-care with same-day results, informing the best treatment course for patients and avoiding the unnecessary use of antibiotics. For example, target analytes may be useful in infection diagnostics of the urinary tract infection and chronic wounds, sepsis diagnostics and microbiome profiling.

The vast majority of microbial identification assays today rely on sample culture to grow enough of the target, unknown organism which can take days or weeks and is prone to false negatives. In time sensitive infections, the critical window to initiate therapy can be as short as six hours, forcing physicians to prescribe broad-spectrum antibiotics preemptively even before confirming a bacterial agent. Unnecessary antibiotic use engenders drug resistance and can result in long term complications for patients and can be avoided with a reliable rapid microbial identification test.

Rapid diagnostic tests conventionally rely on target-specific sensors that are highly specialized to sense one microbe without cross-reactivity. These tests fail to scale for applications where there are dozens to hundreds of microbes of interest which has limited their general clinical utility. However, a suite of technologies has been developed through research in rapid biomarker diagnostics for targeted diagnostics where portable, point-of-care devices with a few on-board sensors are appropriate.

In certain aspects, the present methods can be used to detect large panels of microbes with fewer sensors than previously theoretically possible through the SPoRe algorithm enabling broad-range diagnostics at the point-of-care. Decades of research into point-of-care diagnostics and microfluidics have matured the field of hardware development to provide a suite of tools for processing samples to separate target microbial cells from a background of human cells and other contaminants for downstream signal acquisition from sensors. Given the measurements from these sensors, the present compressed sensing technique offers a new signal processing approach to infer the microbial content of the sample very efficiently. The signal processing method can be coupled with established sensing technologies.

In infection diagnostics, signal sparsity arises because among a library of dozens to hundreds of possible pathogens, only one or a few pathogens are responsible for any given patient's condition. Rather than mixing sensors in with a bulk sample containing biomarkers (e.g., microbial cells or genes), emerging technologies in microfluidics can split a sample into thousands of tiny nanoliter droplets (FIG. 2B). Measurements from each droplet reflect some function of the microbial content in the droplet, and the integer quantities of microbes in droplets follows a Poisson distribution with parameter vector 1 which captures the average content of microbes in the droplets (FIG. 2A). The present SPoRe algorithm estimates the concentration of microbes in the whole sample based on the pooled set of droplet data (FIG. 2C). While the common assumption is that every droplet's content must be solved for individually, SPoRe lets λ be solved for by pooling all of the droplet measurements simultaneously. This distinction uniquely lets SPoRe diagnose many targets even when the content of specific droplets is ambiguous. In diagnostics, the overall concentration λ is all that is clinically relevant, and present algorithm provides advantages in signal recovery by bypassing the presumed need to solve for all of X (Kota et al., 2021).

In particular aspects, the present methods allow for the use of a decreased number of sensors (M<k) which does not merely result in cost savings. Several of the biological sensing methods that are amenable to rapid assays and simple devices are also fundamentally constrained in the number of measurements that can be resolved (Guo et al., 2012; Suea-Ngam et al., 2019). For example, with fluorescence measurements, only M=3 to 5 color channels can be reliably distinguished per reaction. With polymicrobial infections presenting with as many as k=5 microbes (Peters et al., 2012), the present compressed sensing framework enables for a new rapid diagnostic test.

In further aspects, the present methods allow for modular sensing. Most compressed sensing approaches rely on nonspecific linear sensors. For example, a light signal that scales with the microbial content and reacts broadly with multiple microbes. Current options for nonspecific linear sensing have poor sensitivity. The present SPoRe algorithm can perform diagnostics with any sensing mechanism which opens the floodgates for sensors that can couple with the present approach. The present studies showed that SPoRe can recover microbial content even from binary sensors representing the worst-case of information loss from which clinically relevant performance can be achieved.

With the standard compressed sensing framework, microbial species outside of the expected panel of microbes can result in erroneous misclassifications. With conventional target-specific probes, such species result in false negatives. Both situations can be avoided with microfluidic partition measurements as out-of-library species would result in very low measurement likelihoods that can be statistically detected. These samples could be labeled as “unknown.”

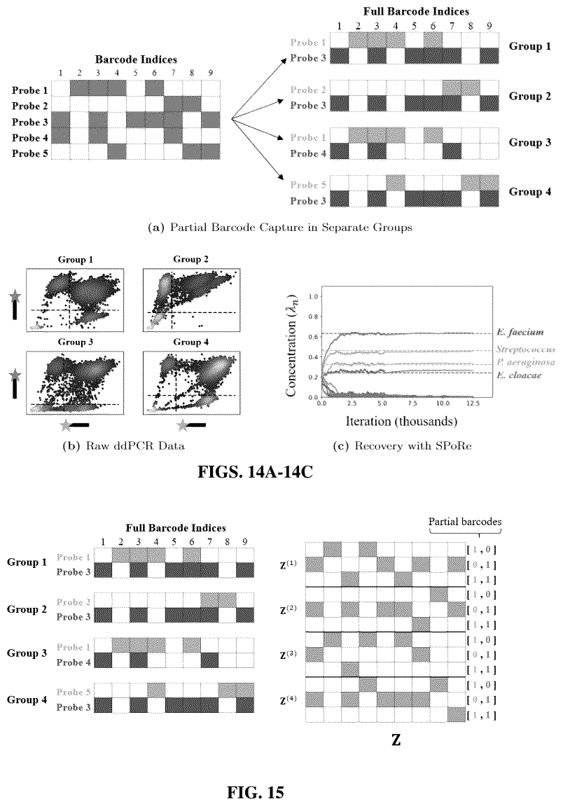

The present experiments demonstrated that even binary measurements from partitions are sufficient to recover microbial quantities. A droplet digital PCR (ddPCR) instrument was used with the universal bacterial 16S gene that exhibits small sequence differences between species. A small set of probes were selected that can capture these differences to assign 9 pathogenic genera their own digital barcode. FIG. 14 illustrates the concept of asynchronous fingerprinting which bypasses the limitations of current systems; although five probes assign unique barcodes to bacteria, only two probes can be sensed per droplet in ddPCR. For each sample, four sets of droplets were generated with two probes and the bacterial quantities were recovered from the pooled data with SPoRe.

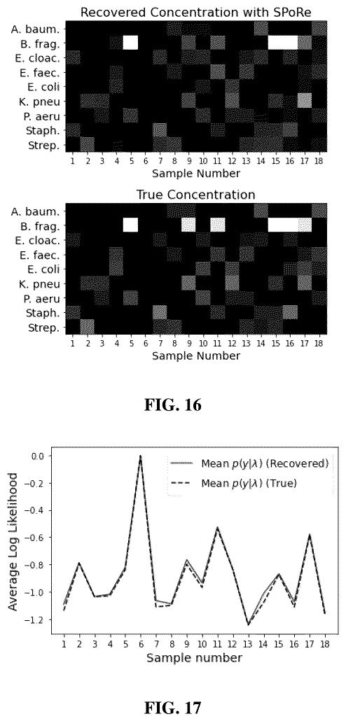

In an initial demonstration with 18 samples, SPoRe recovered the bacterial content with an average cosine similarity of 0.96 (max of 1) which illustrates its ability to capture the relative abundance of bacteria (FIG. 16). SPoRe can thus reliably identify the dominant microbes in a polymicrobial samples, empowering clinicians to select more targeted therapies.

While current methods depend on single-cell capture by diluting samples sufficiently to enforce that non-empty partitions have only one cell with high probability by Poisson statistics (Guo et al., 2012; Velez et al., 2017), SPoRe can tolerate multi-cell capture events to facilitate a broader dynamic range of sample concentrations. Dynamic range is valuable in diagnostics as the sample content is unknown which makes the process of prior dilution blind. For example, the methods may be applied to bacterial, and fungal infection diagnostics. These organisms have genome sizes on the order of 10⁶-10⁸ base pairs of DNA, making them more amenable to the nonspecific genomic DNA sensing methods. Specifically, such DNA sensors can bind a sufficient number of times to each microbe to easily generate unique and robust fingerprint signals.

In some aspects, the present methods comprise the detection of pathogens for the diagnosis of urinary tract infections (UTIs). In the U.S., UTIs are responsible for over 10 million office visits and $3B in healthcare and societal costs (Flores-Mireles et al., 2015). The commercially available rapid diagnostic tests for UTIs cannot sense the variety of pathogens that are encountered in hospitals, necessitating sample culture and prophylactic antibiotics before confirming a bacterial agent (Marscha et al., 2012). There is a clear need for a diagnostic test that can distinguish between the bacterial and fungal pathogens that occur with low but nonetheless significant prevalence. UTI diagnosis generally requires a limit of detection on the order of 10⁴ cells/mL of sample.

In some aspects, fungal pathogens may be detected with the simultaneous PCR amplification of the 16S (bacteria) and 18S (fungi) genes.

Further aspects provides methods for the detection of bacterial biofilms in chronic wounds. Bacterial biofilms in chronic wounds delay healing and require targeted treatment depending on bacterial identification (Wu et al., 2019). Bacterial density in samples tends to be at minimum 10⁴ cells/mL (Woods et al., 2012; Dalton et al., 2011). Current diagnostics are limited by their inability to diagnose multiple species in the same sample, identify the dominant pathogen, or culture the microbes altogether (Wu et al., 2019). Advanced molecular assays rely on 16S for downstream sequencing. The present methods can be used to to characterize the relative abundance of microbes in polymicrobial samples and can bypass the need for downstream sequencing, facilitating rapid species identification for further targeted subtyping. Once the pathogen species are identified, targeted transcriptomics can help further guide antibiotic therapy.

Sepsis Diagnostics: The need for a rapid assay is most urgent in sepsis diagnostics. Sepsis is one of the leading causes of death in the U.S. as improper therapy leads to rapid organ failure. Sepsis is particularly challenging in diagnostics as the limit of detection can be as low as one cell per mL of sample, but the total microbial genomic copies per mL could be on the order of 10²-10³ copies (Bacconi et al., 2014).

Microbiome Community Profiling: The composition of patients' gut microbiome is increasingly linked to health and disease (Clemente et al., 2012; Hall et al., 2017). Quantifying the abundance of hundreds of kinds of microbes in patients' guts is a costly and time intensive process, usually achieved through DNA sequencing. Patients' microbiomes can rapidly change over time via changes in diet or therapy (David et al., 2014; Galloway-Pena et al., 2017), making temporal monitoring clinically useful. Routine monitoring applications would particularly benefit from a rapid, inexpensive test. While sample sparsity is needed for typical compressed sensing and not exhibited in the microbiome, the theory developed in the studies below indicate that sparsity is not necessary for estimating signals with microfluidic partitioning and Poisson constraints.

B. Detecting Out-of-Library Microbes with Microfluidics and Compressed Sensing

In some aspects, the present framework for microbial diagnostics comprises (1) the fingerprinting of microbial biomarkers with nonspecific nucleic acid sensors, (2) the capture of partial or full fingerprint measurements from droplets, and (3) the iterative maximization of a likelihood function by gradient ascent (maximum likelihood estimation, MLE). Specifically, for (3), the microbial concentrations λ_{MLE} can be estimated by maximizing the average log-likelihood of the droplet measurements, where y_{d} represents the measurement(s) from the dth droplet:

$${λ}_{M L E} = arg {max}_{λ} \frac{1}{D} {\sum }_{d = 1}^{D} log p ( {y}_{d} \vert λ ) = arg {max}_{λ} \frac{1}{D} {\sum }_{d = 1}^{D} log {\sum }_{x ∈ {Z}_{+}^{N}} p ( {y}_{d} \vert x ) p ( x \vert λ )$$

Implicit in p(y_{d}|x) is the sensing model from droplet signal x (microbial content) to measurements. The present Sparse Poisson Recovery (SPoRe) algorithm may be used to solve this function with Monte Carlo estimations of the infinite sum over all integer vectors x. Note that if the set of relevant signals is finite, then gradients can be computed exactly. FIG. 2 reviews this overall process.

The dimension N of x covers the N microbes in the library (equivalent to panel, database, etc.). Maximizing this function can return some solution intended to be a diagnostic result. However, this solution can be evaluated for its quality based on its consistency with the observed measurements. This evaluation is important to detect microbes that exhibit fingerprints that are not included in the library—i.e., microbes that are unknown, mutated, or otherwise unaccounted for.

In the general case, the empirical distribution of measurements p(y_{observed}|λ_{MLE}) may be compared with the theoretical distribution of measurements p(y|λ_{MLE}) given the same MLE solution. In the present demonstration with droplet digital PCR (ddPCR), the measurement space is discretized with binary thresholding, so a χ squared goodness of fit test may be used based on the expected and observed measurement counts for each measurement. An exemplary process comprises:
1. For each sample, remove one bacteria from the library by deleting the corresponding column from each sensing matrix.
2. Solve the MLE problem to find λ_{MLE}.
3. Based on the number of droplets measured in each of four groups, calculate the expected number of [0,0], [0,1], [1,0], and [1,1] measurements in each group. This process forms (4 measurements)*(4 groups)=16 bins of measurements to evaluate.
4. Use a χ-squared goodness of fit test to compare the expected number of measurements versus the observed measurements in the 16 bins.
5. Repeat (1-4) for each of the present bacteria in the corresponding sample.
6. Repeat (2-4), skipping the removal step to compute the solution given the full database of bacterial barcodes.

In the present studies, with 5 samples of 2 bacteria, 6 samples of 3 bacteria, and 6 samples of 4 bacteria, there were 69 total cases for which the inventors evaluated χ-squared tests. The ability of the p value from the chi squared test to serve as a discriminator between samples with or without unknown microbial fingerprints was evaluated.

Depending on the nature of the measurements (e.g., discrete vs continuous) and function that maps signals to measurements (e.g., linear vs nonlinear), other statistical tests may be used to determine the presence of an unknown microbe.

C. Methods of Detection

In certain aspects, the present disclosure provides methods for detecting target analytes, such as pathogens or other microbes in a sample, such as for the diagnosis or prognosis of a disease. In some aspects, the present methods comprise selecting a treatment for a disease patient as well as methods of treating a patient with a disease with the selected treatment. Methods of detection may include detecting DNA, such as probes and PCR, or polypeptides.

As used herein, the term “sample” refers to any sample suitable for the detection methods provided by the present invention. The sample may be any sample that includes material suitable for detection or isolation. Sources of samples include blood, pleural fluid, peritoneal fluid, urine, saliva, malignant ascites, broncho-alveolar lavage fluid, synovial fluid, and bronchial washes. In one aspect, the sample is a blood sample, including, for example, whole blood or any fraction or component thereof. A blood sample suitable for use with the present disclosure may be extracted from any source known that includes blood cells or components thereof, such as venous, arterial, peripheral, tissue, cord, and the like. For example, a sample may be obtained and processed using well-known and routine clinical methods (e.g., procedures for drawing and processing whole blood). In one aspect, an exemplary sample may be peripheral blood drawn from a subject with cancer. In some aspects, the biological sample comprises a plurality of cells. In certain aspects, the biological sample comprises fresh or frozen tissue. In specific aspects, the biological sample comprises formalin fixed, paraffin embedded tissue. In some aspects, the biological sample is a tissue biopsy, fine needle aspirate, nipple aspirate, blood, serum, plasma, cerebral spinal fluid, urine, stool, saliva, circulating tumor cells, exosomes, or aspirates and bodily secretions, such as sweat.

The sample could also be from a nonclinical source but could contain microbial content.

These sources could include but are not limited to soil, fermentation reactors, wastewater, and commercial microbial products such as probiotics. Samples could also be swab samples from surfaces in an environment.

“Prognosis” refers to as a prediction of how a patient will progress, and whether there is a chance of recovery. Prognosis may also refer to a nonclinical situation, such as contaminant detection, quality assurance, or general microbiome profiling of various environments.

The sample obtained from an individual may contain cells affected by the disease, meaning that the cells express the disease-associated analyte. Thus, where the analyte is expressed in a cell-specific manner, the sample will contain the cell type in which the disease analyte is expressed.

The sample analyzed may be anybody fluid sample, such as, for example, blood serum, cerebrospinal fluid, mucus, saliva, vaginal secretion, and urine, or may be a sample of the diseased tissue itself.

In some embodiments of the methods described herein, detecting the presence a biomarker in a biological sample obtained from an individual comprises determining the presence of a modified polypeptide in the sample. A polypeptide can be detected by any of a number of means known to those of skill in the art, including analytical biochemical methods, such as electrophoresis, capillary electrophoresis, high performance liquid chromatography (“HPLC”), thin layer chromatography (“TLC”), hyperdiffusion chromatography, and the like, or various immunological methods, such as fluid or gel precipitation reactions, immunodiffusion (single or double), immunoelectrophoresis, radioimmunoassay (“RIA”), enzyme-linked immunosorbent assay (“ELISA”), immunofluorescent assays, flow cytometry, FACS, western blotting, and the like.

Other methods of sensing may be based on cellular responses to environmental changes such as growth media. In these embodiments, the analytes are whole cells rather than individual molecules, genes, or other biomarkers. Different sensing groups may each contain a unique environment such that a cell's fingerprint is its coded in its unique combination of responses to the multiple environments. These responses may be measured by growth rate with metabolic assays such as resorufin-based fluorescence monitoring or turbidity monitoring. In other embodiments, they may involve single-cell RNA sequencing data or nucleotide probe-based measurements of RNA for gene expression analysis.

### III. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice.

However, those of skill in the art should, in light of the present disclosure, appreciate that many changes can be made in the specific embodiments which are disclosed and still obtain a like or similar result without departing from the spirit and scope of the disclosure.

### Example 1—Extreme Compressed Sensing of Poisson Rates from Multiple Measurements

The present studies provides a generally applicable framework for the MMV problem with Poisson signals (MMVP). Let each signal x*_{d} be drawn independently from a multivariate Poisson distribution parametrized by the N-dimensional, k sparse vector λ*. That is, x*_{n,d}˜Poisson(λₙ*) are independent. This framework should not be confused with the well-studied “Poisson compressed sensing” problem in imaging where the measurement noise, rather than the signal, follows a Poisson distribution (Raginsky et al., 2010; Harmany et al., 2012). In contrast to typical MMV problems, the present studies find an estimate \hat{λ}≈λ* from D observations rather than to estimate \hat{X}. Each signal and measurement pair (x*_{d}, y_{d}) is in one of G different sensor groups, each with its own sensing matrix Φ^{(g)} such that y_{d}=Φ^{(g)}x*_{d}. The group g associated with each index d is known and deterministic. This measurement group multiplexing is similar to that found in the single pixel camera (SPC) (Duarte et al., 2008); however, in the SPC the x*_{d} are assumed to be equal, whereas in MMVP they are independently sampled. In microfluidics, several sensor groups can be feasibly achieved by forking an input microfluidic channel into G reaction zones each containing its own set of M sensors. Note that x_{d}*˜Poisson(λ*) regardless of which group it is in. The statement Y=ΦX* is the special case without noise where G=1 and is illustrated in FIG. 5. For multiple groups with X^{(g)}* denoting the submatrix of X* in group g, Y is the following concatenation:

Y=[Φ⁽¹⁾X⁽¹⁾* . . . Φ^{(G)}X^{(G)}*]. (1)

The present studies demonstrate the first exploration of the MMVP problem and develop a novel recovery algorithm and initial theoretical results. a maximum likelihood estimation (MLE) approach was taken, treating Y as a set of D observations from which to infer \hat{λ}. The contributions are 1: the Sparse Poisson Recovery (SPoRe) algorithm that tractably estimates \hat{λ}; 2: theoretical results on the identifiability of the MMVP model, proving that MLE can asymptotically recover λ* even when M=G=1 for any k, and insights into MLE performance; and 3: simulations demonstrating SPoRe's superior performance over existing and custom baseline algorithms. The achievable measurement rate in the MMVP model is unprecedented in CS, and insights were analytically derived into the influence of various system parameters and these insights were confirmed in the simulated experiments. It was found that system designers should first maximize M and then increase G as necessary depending on the expected real-world conditions. It was found that using G>1 was helpful for cases with high noise or high Σₙλ*ₙ.

SPoRe's strong performance even with extremely low M∈{1,2,3} under very high measurement noise uniquely enables sensor-constrained applications in biosensing. Although microfluidics devices can rapidly generate a large number of partitions D at a tunable rate, most optical and electrochemical sensing modalities that can keep pace are limited in M (Guo et al., 2012; Suea-Ngam et al., 2019). Commonly, fluorescently tagged sensors reveal droplets' contents rapidly as they flow by a detector, but spectral overlap generally limits M to be five or less without highly specialized, system-specific approaches (Orth et al., 2018). High M alternatives such as various spectroscopic techniques limit throughput, necessitate additional instrumentation, or complicate workflows (Suea-Ngam et al., 2019, Zhu & Fang, 2013). These severe restrictions in M may have forestalled research into CS's potential role in microfluidics.

Background: The MMVP problem has not yet been explored, likely owing to the ongoing maturation of microfluidics and only recent application of CS to biosensing. The Poisson signal model constrains elements of X to be nonnegative integers under a set of defined probability mass functions. Some aspects of this structure have been studied tangentially, but not the MMVP structure directly.

The core MMV problem only imposes joint sparsity. Early greedy algorithms for this generalized scenario extend the classic Orthogonal Matching Pursuit (OMP) algorithm (Pati et al., 1993) into OMPMMV (Chen & Huo, 2006), simultaneously developed as Simultaneous OMP (S-OMP) (Tropp et al., 2006). Generally, OMP-based algorithms iteratively build a support set of an estimated sparse solution \hat{x} (or \hat{X}) by testing for the correlation between columns of Φ and the residuals between the measurements and previous estimates. A suite of greedy algorithms was recently developed that impose nonnegative constraints to a number of MMV approaches including OMP's analogues, and the nonnegative extensions outperformed their generalized counterparts (Kim & Haldar, 2016).

The application of integer constraints to the SMV problem has proven challenging in the literature. Some theory involving sensing matrix design includes (Dai & Milenkovic, 2009a; Fukshansky et al., 2019), but practical algorithms have required additional constraints on the possible integers, e.g., x∈{0,1}^{N} or other finite-alphabet (Zhu & Giannakis, 2011; Nakarmi & Rahnavard, 2012; Tian et al., 2009; Draper & Malekpour, 2009; Shim et al., 2014). A recent study verified that these problems, as well as those with unbounded integer signals, are NP-hard (Lange et al., 2020). Algorithms for the unconstrained integer SMV problem thus apply greedy heuristics such as OMPbased approaches (Flinth & Kutyniok, 2017; Sparrer & Fischer, 2016).

Additional structural constraints can also make these problems tractable. The communications problem of multi-user detection (MUD), reviewed in (Alam & Zhang, 2018a), bears some similarity to MMVP. Here, the activity of N users is the signal of interest and generally follows a Bernoulli model where each user is active with the same prior probability pa (Zhu & Giannakis, 2011). An alternative prior with Σₙ₌₁^{N}x_{n,d}˜Poisson(λ) models the mean number of total active users in any given signal (Ji et al., 2015) although the authors solely explored an overdetermined system. Applying an MMV framework to MUD enables underdetermined (M<N) applications but has generally assumed that any active user is active for the entire frame of observation (a row of X is entirely zero or nonzero) (Monsees et al., 2014; Liu et al., 2015). Recently, the potency of sensor groups with a G=2 system was demonstrated in the MUD context (Alam & Zhang, 2018b). Despite some similarities to MMVP with an MMV framework and discrete signals, MUD most fundamentally differs from MMVP in its utilization of the probabilistic structure of X. In MUD, the model parameters governing user activities are assumed and leveraged in recovery of X, whereas in MMVP, the model parameters in λ* themselves are the target of recovery.

Regardless of the particular structural constraints imposed in the variants of MMV above, M>k is necessary to recover X* (Davies & Eldar, 2012). However, in MMV models where the rows of X* are statistically independent, the simpler task of support recovery has been proven to be possible below the M>k regime, as low as M=Ω(\sqrt{k}) (Balkan et al., 2014; Pal & Vaidyanathan, 2015; Koochakzadeh et al., 2018). In particular, multiple measurement sparse Bayesian learning (M-SBL) (Balkan et al., 2014) takes a MLE approach in a setting very similar to MMVP, but where x*_{d} have a Gaussian distribution rather than Poisson, and there M=Ω(\sqrt{k}) is limit for sparse support recovery even for D→∞.

In contrast, the present studies prove that in the MMVP problem, this is improved to M≥1 (i.e., no minimum number of measurements) regardless of k when D→∞, even when G=1. Furthermore, the MLE in this setting recovers λ* exactly. It was demonstrated empirically that λ* can be recovered well for finite D even for extremely low M such as M∈{1,2,3}.

Sparse Poisson Recovery (Spore) Algorithm

Notation: A probability mass function is denoted by P(⋅) and a probability density function by p(⋅). R^{N} and Z^{N} are used to represent the N-dimensional Euclidean space and integer lattice, respectively. [symbol] ₊^{N} and [symbol] ₊^{N} denote the non-negative restrictions on these spaces. Script letters ( [symbol] . . . ) are used for sets unless otherwise described. Lowercase and uppercase boldface letters are used for vectors and matrices, respectively. Their dimensions are represented with uppercase letters (e.g., X∈ [symbol] ₊^{N×D}) that are indexed by their lowercase counterparts. For example, x_{n,d} is the element of X in the nth row and dth column, and the inventors use the shorthands xₙ and x_{d} to represent the entire nth row and dth column vectors, respectively. Other lower case letters (a, b, ϵ, etc.) may represent variable or constant scalars depending on context. λ* and X* are used to refer to the true signal values, and estimates are denoted \hat{λ} and \hat{X} with the source of the estimate (e.g., MLE, SPoRe, baseline algorithm) being implicit from the context. ∥⋅∥ is used to denote a norm, ∥⋅∥₀ for the number of nonzero elements of a vector, ∥⋅∥₁ for the [symbol] ₁ vector norm, ∥⋅∥₂ for the Euclidean norm, and ∥⋅∥_{F} for the Frobenius norm. ∥X∥_{Rx} [symbol] Σₙ max_{d}|x_{n,d}| is also used, relaxation of the row [symbol] ₀ quasi-norm defined in (Tropp, 2006). The null space of matrix A is denoted by [symbol] (A). As one abuse of [symbol] notation, for densities of the form p(y|x), the corresponding Φ^{(g)} applied to x and the relevant noise model are implicit. Also, the division of two vectors represent element-wise division.

Algorithm. If the index d is in sensor group g, the inventors say that linear measurements are corrupted by an additive random noise vector b_{d}:

y_{d}=Φ^{(g)}x_{d}+b_{d}. (2)

b_{d} was entirely independent (e.g., additive white Gaussian noise (AWGN), as used in the simulations) or dependent on x. With x_{d}˜Poisson(λ*) i.i.d., y_{d} are independent across d as well. The MLE estimate maximizes the average log likelihood of the measurements:

$${\hat{λ}}_{MLE} = {arg max}_{λ} {\prod }_{d = 1}^{D} p ( {y}_{d} \vert λ ) \qquad ( 3 )$$

$$= {arg max}_{λ} \frac{1}{D} {\sum }_{d = 1}^{D} log {\sum }_{x ∈ {ℤ}_{+}^{N}} p ( {y}_{d} \vert x ) P ( x \vert λ ) . \qquad ( 4 )$$

Because the infinite sum over x yields an intractable posterior distribution, the popular expectation maximization (EM) algorithm (Dempster et al., 1977) cannot be applied to solve this MLE problem. Instead, the Sparse Poisson Recovery (SPoRe) algorithm (Algorithm 1) optimizes this function with batch stochastic gradient ascent, drawing B elements uniformly with replacement from {1, . . . , D} to populate a batch set [symbol] . First, note that

$${∇}_{λ} P ( x \vert λ ) = P ( x \vert λ ) ( \frac{x}{λ} - 1 ) . \qquad ( 5 )$$

Denoting the objective function from the right-hand side of (4) as [symbol] , the gradient is

$${∇}_{λ} ℓ = \frac{1}{B} \frac{{\sum }_{x ∈ {ℤ}_{+}^{N}} p ( {y}_{d} \vert x ) P ( x \vert λ ) x}{λ {\sum }_{x ∈ {ℤ}_{+}^{N}} p ( {y}_{d} \vert x ) P ( x \vert λ )} - 1. \qquad ( 6 )$$

With gradient ascent, each iteration updates λ←λ+α∇_{λ} [symbol] with learning rate α. However, the summations over all of [symbol] ₊^{N} are clearly intractable. SPoRe approximates these quantities with a Monte Carlo (MC) integration over S samples of x, newly drawn for each batch gradient step from sampling distribution Q: [symbol] ₊^{N}→ [symbol] ₊, such that

$${\sum }_{x ∈ {ℤ}_{+}^{N}} p ( y \vert x ) P ( x \vert λ ) \approx \frac{1}{S} {\sum }_{s = 1}^{S} \frac{p ( y \vert {x}_{s} ) P ( {x}_{s} \vert λ )}{Q ( {x}_{s} )} . \qquad ( 7 )$$

The optimal choice of Q(xₛ) is beyond the scope of this work, but it was found that Q(xₛ)=P(xₛ|λ) simplifies the expression, is effective in practice, and draws inspiration from the EM algorithm. In other words, the sampling function is updated at each iteration based on the current estimate of λ. The gradient thus simplifies to

$${∇}_{λ} ℓ = \frac{1}{B} {\sum }_{d ∈ ℬ} \frac{{\sum }_{s = 1}^{S} p ( {y}_{d} \vert {x}_{s} ) {x}_{s}}{λ {\sum }_{s = 1}^{S} p ( {y}_{d} \vert {x}_{s} )} - 1. \qquad ( 8 )$$

Note that if only one \hat{x}_{d}∈ [symbol] ₊^{N} satisfied p((y_{d}|\hat{x}_{d})>0 for every y_{d}, the objective [symbol] would be concave with

$$\hat{λ} = \frac{1}{D} {\sum }_{d = 1}^{D} {\hat{x}}_{d} ,$$

i.e., the MLE solution if X* were directly observed. Of course, with compressed measurements and noise, multiple signals may vie to “explain” any single measurement, but SPoRe's key strength is that it jointly considers independent measurements to directly estimate \hat{λ}.

It is noted that for finite samples, since the MC integration occurs inside a logarithm, the stochastic gradient is biased. However, since it converges in probability to the true gradient, results can be expected to be comparable to SGD with an unbiased gradient for sufficiently large S (Chen & Luss, 2019).

FIG. 6 illustrates key concepts of SPoRe and MMVP with a small example where M=G=1 and λ*=[0.5,0,0.5] for which p(y|λ) can be numerically computed for various λ. The measurements y_{d} are effectively drawn from an underlying mixture distribution depending on the noise; e.g., under AWGN, y_{d} follows a Gaussian mixture. The weights on each mixture component are controlled by λ. In simulated recovery, SPoRe assigns weights to the mixture via \hat{λ} according to the distribution of measurements, coming close to the true underlying distribution. In contrast, an [symbol] ₁-Oracle which represents best-case performance for a standard, convex sparse recovery process fails because M<k as shown by its error in λ and illustrated by the difference in the distributions. Moreover, by using Φ=[1,2,3], many x will map to the same y. While CS theory generally focuses on conditions for unique or well-spaced projections of k-sparse signals (e.g., the restricted isometry property, RIP (Candes & Tao, 2005), it was demonstrated that such restrictions are unnecessary in MMVP. By accounting for the latent Poisson distribution in the signals, SPoRe succeeds even when M<k.

Algorithm 1 summarizes the implementation details of SPoRe. Even though λ∈ [symbol] ₊^{N}, λ≥ϵ can be enforced by clipping (ϵ=10⁻³ in the simulations) to maintain exploration of the parameter space. Note that in (8), gradients can become very large with finite sampling as some elements of λ approach zero. It was found that rescaling gradients to maximum norm γ helps stabilize convergence. For rescaling, only the subvector δ_{Γ} of the α-scaled gradient δ was considered, defining indices n∈Γ⊆{1, . . . , N} if λₙ+δₙ>ϵ. This restriction ensured that rescaling is solely based on the indices still being optimized, excluding those clipping to ϵ.

Algorithm 1 Sparse Poisson Recovery (SPoRe)

| Input: λ⁽⁰⁾, B, S, γ, α, ϵ | |
|---|---|
| 1: λ ← λ⁽⁰⁾ | |
| 2: i = 0 | |
| 3: repeat | |
| 4: Draw B columns of Y uniformly with replacement | |
| 5: Draw S new samples from Q(xₛ) | |
| 6: δ ← α∇_{λ}l(λ) [symbol] (8) | |
| 7: if ∥δ_{Γ}∥₂ > γ then | |
| $8 : δ ← \frac{γ}{{‖ {δ}_{Γ} ‖}_{2}} δ$ | [symbol] Rescale gradient step |

9: end if
10: λₙ ← max(λₙ + δₙ, ϵ)
11: until stopping criterion met
12: return λ

For the stopping criterion, a moving average of \hat{λ} was evaluated for convergence. The median of p(y_{d}|λ) was also tracked for d∈ [symbol] , accounting for stochasticity in likelihood approximations and batch selections such that may reduce α if no improvements in the median have been seen within a patience window and terminate if α is reduced five times. All experiments were conducted on commodity personal computing hardware. Ultimately, recovery of \hat{λ} takes a few minutes on a single core for the problem sizes considered (M≤15, N≤50), and SPoRe can be easily parallelized for faster performance.

Within an iteration, it was found that using the same S=1000 samples for all d∈ [symbol] helped to vectorize the implementation to dramatically improved speed over sampling S times for each drawn y_{d}. This simplification had no noticeable influence on performance. While random initializations were found with a small offset λ⁽⁰⁾˜Uniform(0,1)+v (with v=0.1) to be effective in general, a numerical issue was encountered when under low-variance AWGN. Even though AWGN results in nonzero probabilities everywhere, p(y_{d}|xₛ) may numerically evaluate to zero for all drawn samples in low-noise settings. These zeros across all samples result in undefined terms in the summation over d∈ [symbol] in (8). SPoRe simply ignores such undefined terms, but when this numerical issue occurs for all of [symbol] , SPoRe takes no gradient step. With very low noise and large N dampening the effectiveness of random sampling, SPoRe may stop prematurely as it appears to have converged. This problem did not arise with larger noise variances where even inexact samples pushed \hat{λ} in the generally appropriate direction until better samples could be drawn (recall that Q(x)=P(x|\hat{λ}) at each iteration). Nonetheless, it was decided to set λ⁽⁰⁾=v for consistency across all simulated experiments. It was speculated that setting λ⁽⁰⁾ to a small value helped encourage sampling sparse x's in early iterations to help find xₛ with nonzero p(y_{d}|xₛ), bypassing the numerical issue altogether.

Theory and analysis. The summation over x inside the logarithm of the objective function complicates the precise analysis of the SPoRe algorithm. However, the asymptotic MMVP problem can be considered as D→∞ and its MLE solution to gain insight into the superior recovery performance of SPoRe. In this setting, the powerful result was obtained that λ* is exactly recoverable even when M=G=1 under a simple null space condition on Φ. The loss in Fisher Information was then characterized for MMVP and showed how losses accrue with the increase of signals that map to the same measurements, an effect that increases with k. Lastly, insights were derived into the influence of sensor groups through a small-scale analysis. From a system design standpoint, it was found that designers should first increase M as much as feasible and then increase G as needed. All proofs can be found in the Appendix below.

Identifiability of MMVP Models. The primary theoretical result was that the asymptotic MLE of the MMVP problem is exactly equal to λ* as long as a simple null space condition on Φ was satisfied. This result improved upon the M=Ω(\sqrt{k}) limitations of correlation-aware support recovery methods (Balkan et al., 2014; Koochakzadeh et al., 2018), placing no restrictions on k and M. The reason for this improvement is that Φ transforms an integer lattice rather than a linear subspace such that the resulting model is still identifiable.

Identifiability refers to the uniqueness of the model parameters that can give rise to a distribution of observations. A model [symbol] {p(⋅|λ):λ∈ [symbol] ₊^{N}} is a collection of distribution functions which are indexed by the parameter λ; in the MMVP problem, each choice of Φ and noise give rise to a different model [symbol] . Through an optimization lens, if the inventors' model is identifiable, then λ* is the unique global optimum of the data likelihood as D→∞. Recall that

$$p ( y \vert λ ) = {Σ}_{x ∈ {ℤ}_{+}^{N}} p ( y \vert x ) P ( x \vert λ ) ,$$

meaning that this model can be interpreted as each sensor group consisting of a mixture whose elements' positions are governed by Φ^{(g)}x, distributions by the noise model, and weights by P(x|λ). in this analysis on G=1 was focused on, since as D→∞, at least one sensor group contains infinite measurements. If the corresponding Φ^{(g)} satisfies the conditions described here, then the model is identifiable. Formally:

Definition III.1 (Identifiability). The model [symbol] is identifiable if p(y|λ)=p(y|λ′) ∀y⇒λ=λ′ for all λ, λ′∈ [symbol] ₊^{N}.

The identifiability of mixtures is well-studied (Teicher, 1963; Tallis 1969); if a mixture is identifiable, the mixture weights uniquely parametrize possible distributions. For finite mixtures, a broad set of distributions including multivariate exponential and Gaussian have been proven to be identifiable (Yakowitz & Spragins, 1968). A finite case may manifest in realistic MMVP systems where measurements y must eventually saturate; all sensors have a finite dynamic range of values they can capture. In the most general case, p(⋅|λ) is a countably infinite mixture.

Although less studied, countably infinite mixtures are identifiable under some classes of distributions (Yang & Wu, 2013). The AWGN that the inventors use in the inventors' simulations is identifiable for both the finite and countably infinite cases. Characterizing the full family of noise models that are identifiable under countably infinite mixtures is beyond the scope of this work. The contribution was that given a noise model that yields identifiable mixtures, equal mixture weights induced by λ and λ′ imply λ=λ′. the sufficiency of the following simple conditions on Φ for identifiability were proven:

Theorem III.2 (Identifiability of Mixture Weights). Let b be additive noise drawn from a [symbol] distribution for which a countably infinite mixture is identifiable. If [symbol] (Φ)∩ [symbol] ₊^{N}={0} and ϕₙ*≠ϕ_{n′} ∀n,n′∈{1, . . . , N} with n≠n′, then [symbol] is identifiable.

The null space condition essentially says that any nonzero vector in [symbol] (Φ) must contain both positive and negative elements. Many practical Φ satisfy this constraint (e.g., any Φ with at least one strictly negative or positive row). The second condition is trivial: no two columns of Φ can be identical. A separate sufficient condition was also obtained, that Φ drawn from any continuous distribution results in identifiability.

Corollary III.3 (Identifiability with Random Continuous Φ). Let b be additive noise drawn from a distribution for which a countably infinite mixture is identifiable. If the elements of Φ are independently drawn from any continuous distribution, then [symbol] is identifiable.

The general result of Theorem III.2 was emphasized, since discrete sensing is common in biomedical systems. For example, sensors are often designed to bind to an integer number of known target sites and yield “digital” measurements (Basu, 2017; Zhang et al., 2012). Discrete Φ can give rise to what the inventors call collisions. Formally:

Definition III.4 (Collisions and Collision Sets). Let Φ∈R^{M×N} be a sensing matrix applied to signals x∈ [symbol] ₊^{N}. A collision occurs between x and x′ when Φx=Φx′. A collision set for an arbitrary u∈ [symbol] ₊^{N} is the set [symbol] ᵤ={x:Φx=Φu; x∈ [symbol] ₊^{N}}.

If the distribution from which b is drawn is fixed (e.g., AWGN) or a function of Φx, then the mixture weights are the probability mass of each collision set. Let the set of collision sets be [symbol] with [symbol] ᵤ∈ [symbol] being an arbitrary collision set.

$$p ( y \vert λ ) = p ( y \vert x ∈ {𝒞}_{u} ) P ( {𝒞}_{u} \vert λ ) \qquad ( 9 )$$

$$P ( {𝒞}_{u} \vert λ ) = {\sum }_{x ∈ {𝒞}_{u}} P ( x \vert λ ) . \qquad ( 10 )$$

The weights of the mixture elements are governed by P( [symbol] ᵤ|λ). Given a noise model that yields identifiable mixtures, the same distribution of observations y implies that the mixture weights are identical, i.e., P( [symbol] ᵤ|λ)=P( [symbol] ᵤ|λ′)∀u. It was proven that P( [symbol] ᵤ|λ)=P( [symbol] ᵤ|λ′)∀u implies λ=λ′, which implies the identifiability of [symbol] under both the conditions of Theorem III.2 and Corollary III.3.

The proofs were based on the existence and implications of single-vector collison sets [symbol] ₓ={x}. When (9) holds, u indexes both the mixture elements and the collision sets. In the general case where b is dependent on x and not simply Φx, signals participating in the same mixture element may have different noise distributions. These differences can only further subdivide collision sets and leaves single-vector collision sets unaffected. Thus, the results also covered the general noise case.

Fisher Information of MMVP Measurements. While identifiability confirms that λ* is a unique global optimum of the MLE problem given infinite observations, Fisher Information helps characterize estimation of λ* as D increases. The Fisher Information matrix [symbol] is the (negative) Hessian of the expected log-likelihood function at the optimum λ*, and it is well-known that under a few technical conditions the MLE solution is asymptotically Gaussian with covariance [symbol] ⁻¹/D. Intuitively, higher Fisher Information implies a “sharper” optimum that needs fewer observations for stable recovery. For direct observations of Poisson signals x*_{d} rather than y_{d}, [symbol] is diagonal with [symbol] _{n,n}=1/λ*ₙ. In MMVP with observations of noisy projections (y_{d}), [symbol] and its inverse are difficult to analyze. The reduction in [symbol] _{n,n} in MMVP caused by the noisy measurement of x*_{d} can be characterized and was derived that was empirically confirmed. Concretely, elements of [symbol] follow

$${i , j}_{} = 𝔼 [ ( \frac{\partial }{\partial {λ}_{i}^{*}} log p ( y \vert {λ}^{*} ) ) ( \frac{\partial }{\partial {λ}_{j}^{*}} log p ( y \vert {λ}^{*} ) ) ] . \qquad ( 11 )$$

The shorthand wₓ [symbol] p(y|x)P(x|λ) was denoted and noted that Σₓwₓ=p(y|λ). Following a similar derivation for the partial derivatives in (8), it was shown that the general expression for diagonal elements [symbol] _{n,n} was

$${n , n}_{} = \int {( \frac{{\sum }_{x} {w}_{x} {x}_{n}}{{\sum }_{x} {w}_{x} {λ}_{n}^{*}} - 1 )}^{2} ( {\sum }_{x} {w}_{x} ) dy . \qquad ( 12 )$$

In the ideal scenario, it was observed x*_{d} directly such that

$${n , n}_{ideal}^{} = {\sum }_{x} P ( x \vert λ ) {( \frac{{x}_{n}}{{λ}_{n}} - 1 )}^{2} ( \int p ( y \vert x ) dy ) . \qquad ( 13 )$$

It can be easily shown that (13) reduces to the canonical 1/λ*ₙ. The integration of p(y|x) evaluates to one, but was manipulated algebraically to re-express the quantity as

$${n , n}_{ideal}^{} = \int {\sum }_{x} [ {{w}_{x} ( \frac{{x}_{n}}{{λ}_{n}} - 1 )}^{2} ] dy . \qquad ( 14 )$$

Let [symbol] ₙ^{loss} [symbol] [symbol] _{n,n}^{ideal}− [symbol] _{n,n} and let Σ_{(x,χ)} denote the sum over all pairs of signals x′, χ∈ [symbol] ₊^{N}. Expanding Equations (12) and (14) and simplifying yields

$$\text{?} = \frac{1}{{λ}_{n}^{{*}^{2}}} \int ( {\sum }_{x} {w}_{x} {x}_{n}^{2} - \frac{{( {\sum }_{x} {w}_{x} {x}_{n} )}^{2}}{{\sum }_{x} {w}_{x}} ) dy = \frac{1}{{λ}_{n}^{{*}^{2}}} \int \frac{1}{{\sum }_{x} {w}_{x}} ( \text{?} {w}_{x} , {{w}_{χ} ( {x}_{n}^{′} - {χ}_{n} )}^{2} ) dy . \qquad ( 15 )$$

$$\text{?} \text{indicates text missing or illegible when filed}$$

Note that [symbol] ₙ^{loss} is non-negative such that [symbol] _{n,n}≤ [symbol] _{n,n}^{ideal} and that pairs of signals with x′ₙ≠χₙ can contribute to [symbol] ₙ^{loss}. Also note that, wₓw_{w}=p(y|x′)p(y|χ)P(x′|λ*)P(χ|λ*) and that P(x|λ*)>0 only when supp(x)⊆supp(λ*). Thus, the Fisher Information is only reduced over the direct Poisson observation case when there are pairs of signals that are well-explained by the same y and by the same λ*. Clearly, λ* with higher k resulted in more of such pairs, and this influence of k on Fisher Information was confirmed empirically. Although further precise analysis via Fisher Information was challenging, a deeper analysis of the special case of the MMVP problem with small λ through a different lens was performed.

Small scale analysis. With identifiability, it was known that λ* uniquely maximizes the expected log-likelihood. However, because SPoRe uses stochastic gradient ascent to optimize the empirical loglikelihood, it will typically achieve a \hat{λ} that is near but not equal to λ*. How the neighborhood of λ* changes given the parameters of the problem was therefore studied. The natural way to do this for MLE problems was to consider the Fisher Information matrix as in the previous section, but the presence of a sum inside the logarithm makes analysis difficult. Instead, a particular \hat{λ} near λ* was considered that solves an optimization related to the original likelihood maximization problem. To further simplify the setting, it was considered that the “small scale” case where Σₙλ*ₙ is small enough that there is almost never a case where Σₙx*ₙ>1. It was emphasized that although this setting is simple, the MLE approach can still drastically outperform a trivial solution such as \hat{λ}=E[\hat{x}], where \hat{x}=argmaxₓ p(y|x), since with sufficient noise, x≠x* with arbitrary probability.

At the small scale, the distribution of each x*n becomes Bernoulli with parameter λₙ, and the probability that x*ₙ=1 and x*ₙ, for n≠n′ vanishes. Let n*≙(the first nonzero index of x*, 0 if none), which has a categorical distribution with parameter λ*. Notation was abused so that ϕ₀=0, λ₀* is the probability that n*=0, and Σₙ₌₀^{N}λₙ*=1. Applying Jensen's inequality to the log-likelihood for the conditional expectation given n*,

$$𝔼 [ log {\sum }_{n = 0}^{N} p ( y \vert n ) {λ}_{n} ] \leq {𝔼}_{{n}^{*}} [ log {\sum }_{n = 0}^{N} {𝔼}_{y \vert {n}^{*}} \vert p ( y \vert n ) \vert {λ}_{n} ] . \qquad ( 16 )$$

was obtained. The right-hand side of this inequality was called the Jensen bound. This Jensen bound via the logarithm has the attractive property of having a gradient that is equal to a first-order Taylor approximation of the gradient of the original likelihood (The Taylor expansion is of ƒ(u,v)=u/v, for which a first-order approximation yields E[U/V]≈E[U]/E[V] for random variables U, V). To see this, consider the partial derivatives for a single λₙ:

$$𝔼 [ \frac{p ( y \vert n )}{{\sum }_{{n}^{′} = 0}^{N} p ( y \vert {n}^{′} ) {λ}_{{n}^{′}}} ] \approx {𝔼}_{{n}^{*}} [ \frac{{𝔼}_{y \vert {n}^{*}} [ p ( y \vert n ) ]}{{\sum }_{{n}^{′} = 0}^{N} {𝔼}_{y \vert {n}^{*}} [ p ( y \vert {n}^{′} ) ] {λ}_{{n}^{′}}} ] . \qquad ( 17 )$$

Thus, the optimizer of the Jensen bound was expected to be close to λ* (this is particularly true as measurement noise vanishes and the bound becomes tight).

In the case where G=1 under AWGN, the following result characterizing the solution of the Jensen bound was found. Proposition III.5. If y˜N(ϕ_{n*},n,σ²I) and

$$K = {( exp \{ - \frac{1}{4 {σ}^{2}} {‖ {ϕ}_{n} - {ϕ}_{{n}^{′}} ‖}_{2}^{2} \} )}_{n , {n}^{′} = 0}^{N} \qquad ( 18 )$$

is invertible, then the maximizer \tilde{λ} of the Jensen bound satisfies

$$\tilde{λ} ∝ {K}^{- 1} ( \frac{{λ}^{*}}{{K}^{- 1} ( s - μ )} ) . \qquad ( 19 )$$

where s∈∂∥\tilde{λ}∥₁ and for all n, μₙ≥0 and μₙ\tilde{λ}ₙ=0.

In the case where all entries of \tilde{λ} are positive, s−μ=1. K has values of one along the diagonal and smaller values off the diagonal, so it mimics the identity matrix. Clearly, as K→I, \tilde{λ}→λ*. However, given nonzero σ², K is bounded away from i. Furthermore, since it is impossible to find a set of more than M+1 equidistant points in [symbol] ^{M}, the off-diagonal values of K will differ when M<N, introducing distortion in the transformation.

However, even if M<N, if y is a measurement from a random sensor group, then the effect of this distortion can be mitigated such that \tilde{λ} is a reliable estimator of λ* from a support recovery perspective:

Theorem III.6. If y˜N(ϕ_{n*}^{(g)}, σ²I), g is distributed uniformly, and ϕₙ^{(g)}˜N(0, I) i.i.d., then if G→∞ and all elements of the maximizer i of the Jensen bound are strictly positive, there exist c₁>0,c₂∈ [symbol] such that \tilde{λ}ₙ=c₁λₙ*+c₂ for 1≤n≤N.

If \hat{λ} has the same rank ordering as λ*, the exact support can be recovered. Therefore, an increase in G was expected to improve performance in tasks such as support recovery. From this result, however, gains were expected due to increasing G to be less immediate than those due to increasing M. To see this, the asymptotic nature of Theorem III.6 in G was contrasted with the fact that for a finite choice of M (specifically M=N) all ϕₙ equidistant (or that for M even smaller the inventors can select Φ satisfying a RIP with some acceptable distortion) were selected and the same reliability result was obtained.

In this section, comparisons of SPoRe are presented against existing and custom baseline algorithms and follow with focused experimentation on SPoRe's performance and limitations. For most baseline algorithms that find an estimate R, their estimates were set

$$\hat{λ} = \frac{1}{D} {\sum }_{d = 1}^{D} {\hat{x}}_{d} ,$$

i.e., the canonical Poisson MLE if X* were observed directly. For a performance metric, cosine similarity between \hat{λ} and λ* was chosen as it captures the relative distribution of elements of the solution which was believed most useful to a user in biosensing applications. Although comparisons of cosine similarity mask differences in magnitude, estimates with high cosine similarity also exhibited low mean-squared error. Cosine similarity was plotted alone for brevity. In all simulations, AWGN was used and ϕ_{m,n}^{(g)}˜Uniform(0,1) i.i.d. set, since many sensors are restricted to nonnegative measurements. For each parameter combination, the inventors evaluate over 50 trials in which they draw new Φ^{(g)} and λ* for each trial. Due to high performance variability for some baseline algorithms, all error bars were scaled to ±½ standard deviation for consistency and readability.

Comparison against existing baselines. With no existing algorithm designed for Poisson signals, it was compared against a number of algorithms with various relevant structural assumptions. It was compared against both greedy and convex optimization approaches. First, DCS-SOMP (Baron et al., 2009) was used, a generalization of the common baseline Simultaneous Orthogonal Matching Pursuit (S-OMP) (Tropp et al., 2006) that assumes no structure and greedily solves MMV problems for any value of G. Next, NNS-SP and NNS-CoSaMP (Kim & Haldar, 2016) was used, two greedy algorithms for nonnegative MMV CS motivated by subspace pursuit (SP) (Dai & Milenkovic, 2009b) and compressive sampling matching pursuit (CoSaMP) (Needell & Tropp, 2009) which exhibited the best empirical performance in (Kim & Haldar, 2016). For integer-based recovery, PROMP (Flinth & Kutyniok, 2017) was used, an SMV algorithm for unbounded integer sparse recovery, to recover an estimate for each signal x_{d}. Two support recovery algorithms were also used with M=Ω(\sqrt{k}) measurement rates: M-SBL (Balkan et al., 2014) and a convex, [symbol] ₁-based variance recovery algorithm (Pal & Vaidyanathan, 2015). Support recovery is generally not quantitative and appears to make cosine similarity an inappropriate metric for comparison. However, these algorithms produced an estimate of the variance of x*ₙ, which equals λₙ* for the Poisson distribution, making them coincidentally reasonable baseline approaches for MMVP directly.

For comparison against best-possible performance of the baselines and to avoid hyperparameter search where possible (for regularization weights, stopping criteria, etc.), the baselines were armed with relevant oracle knowledge of λ* or X*. While NNS-SP and NNS-CoSaMP require k as an input, DCS-SOMP and PROMP were also given, algorithms that iteratively and irreversibly select support elements, knowledge of k and have them stop after k elements have been chosen. Additionally, three oracle-enabled convex algorithms were created that minimize Σ_{g=1}^{G}∥Y−Φ^{(g)}X^{(g)}∥_{F} under norm constraints with oracle knowledge of the value of the norm. The [symbol] ₁ norm is commonly used as a penalty for convex solvers to encourage sparsity in sparse recovery. The [symbol] ₁-Oracles included SMV and MMV versions, where in the SMV case, Y was collapsed to a single vector by summing Σ_{d}y_{d}, and a vector R was recovered from which \hat{λ}=\hat{X}/D. The [symbol] ₁-Oracle SMV and [symbol] ₁-Oracle MMV were both given Σ_{n,d}x*_{n,d}. In (Tropp, 2006), ∥X∥_{Rx} was suggested as a better alternative for MMV, so that the Rx-Oracle was given ∥X*∥_{Rx}. In the support recovery algorithm from (Pal & Vaidyanathan, 2015), the vector of sample variances (Var(xₙ))ₙ₌₁^{N} was recovered under a constraint on Σₙ Var(xₙ). Because the sum of variances was provided, this method was called the ΣVar-Oracle. Nonnegativity of the optimization variables was also enforced in all convex problems, which was solve using the convex optimization package CVX in Matlab (Grant & Boyd, 2014; Grant & Boyd, 2008). No oracle knowledge was used for M-SBL, but the same initialization was used as for SPoRe and run its fixed-point update until convergence.

From FIG. 7A, the result that the M<k regime was seen that was only feasible with SPoRe, while conventional CS algorithms, both SMV and MMV, fail. Such a result was expected; generally speaking, CS algorithms seek to minimize measurement error (∥Y−ΦX∥_{F}) while constraining the sparsity of the recovered solution. CS theory focuses on M>k since if M<k, M×k submatrices of Φ yield underdetermined systems in general. In other words, there simply cannot be unique k-sparse minimizers of measurement error alone with M<k, so the conventional CS problem is not well-posed, unlike in the MMVP problem. Support recovery algorithms M-SBL and the ΣVar-Oracle exhibit improved performance over other CS baselines as M decreases, but SPoRe remains far superior as M→1. Next, in FIG. 7B, M=10 was set, a regime where most baselines performed nearly perfectly according to (FIG. 7A), and the AWGN variance was increased. It was seen that even in the conventional regime of N>M>k, SPoRe exhibits the highest noise tolerance which reflects the fact that its leverage of the Poisson assumption minimizes its dependence on accurate measurements. Lastly, however, in FIG. 7C, SPoRe has the unique disadvantage of struggling to recover cases with high Σₙλₙ*. It was observed that as Σₙλₙ* increases, SPoRe's finite sampling results in few to no gradient steps taken as “good” samples with nonzero (numerically) p(y|xₛ) were drawn increasingly rarely, and SPoRe mistakenly terminates. Under AWGN, larger λₙ* raises the signal-to-noise ratio but can paradoxically compromise SPoRe's performance. If M>>k is a practical design choice, practitioners should consider existing MMV approaches if Σₙλₙ* may be highly variable.

Comparison against integer baselines: M<kL In the M<k regime, since with high probability the elements of X* can be bound, the discrete nature of the problem might be expected to admit at the least a brute-force solution for obtaining R that can be used to obtain \hat{λ}. Indeed, if measurement noise is low, then the integer signal that minimizes measurement error for y_{d} is likely to be x*_{d}. But a finite search space alone has not enabled integer-constrained CS research to achieve M<k in general.

One may wonder whether SPoRe is simply taking advantage of this practically finite search space and, by virtue of MC sampling over thousands of iterations, is effectively finding the right solution by brute force. To address this possibility, it was compared against an [symbol] ₀-Oracle that was given k and the maximum value in X* in order to test all

$$( \begin{matrix}N \\ k\end{matrix} )$$

combinations of X's support. For each combination, it enumerates the (max(X*)+1)^{k} possibilities for each x_{d} and selects \hat{x}_{d}=argminₓ∥y_{d}−Φx∥₂. Finally, it returns the k-sparse solution with the lowest minimized measurement error. This algorithm is the only Poisson-free approach in this section.

Comparing SPoRe and other Poisson-enabled baselines against the [symbol] ₀-Oracle characterized the effect of incorporating the Poisson assumption on recovery performance. An early solution of ours for tackling MMVP, which was used as a baseline, was an alternating optimization framework to update estimates of

$$\hat{X} = arg {max}_{X} p ( X \vert Y , \hat{λ} ) and \hat{λ} = arg {max}_{λ} p ( λ \vert Y , X ) .$$

Noting that p(X|Y,λ)∝p(Y|X)p(X|\hat{λ}), this MAP framework for solving for X under AWGN with variance σ² was

$$\begin{matrix}\hat{X} & = & {argmax}_{X} \frac{1}{D} {\sum }_{d = 1}^{D} log P ( {y}_{d} \vert {x}_{d} ) + log p ( {x}_{d} \vert \hat{λ} ) & ( 20 ) \\ & = & {argmax}_{X} \frac{1}{D} {\sum }_{d = 1}^{D} [ - \frac{1}{2 {σ}^{2}} {‖ {y}_{d} - Φ {x}_{d} ‖}_{2}^{2} + & ( 21 ) \\ & & {\sum }_{n = 1}^{N} ( {x}_{n , d} log {\hat{λ}}_{n} - log Γ ( {x}_{n , d} + 1 ) ) ] , & \end{matrix}$$

where the Gamma function Γ(⋅) is the continuous extension of the factorial (Γ(x_{n,d}+1)=x_{n,d}!) and is log-concave in the space of positive reals [symbol] _{+,+}^{N}. the classic branch-and-bound (BB) algorithm was implemented (Land & Doig, 1960) to find the optimal integer-valued solution \hat{X} of the concave objective. Once an estimate \hat{X} was available, the update to \hat{λ} was also concaved with the closed form solution

$$\hat{λ} = \frac{1}{D} {\sum }_{d} {\hat{x}}_{d} .$$

The biconcavity of this objective function in X and λ makes this approach attractive, but it is unclear how to best initialize \hat{λ}. this alternating baseline algorithm was referred to with the prefix “Alt” followed by the method of initialization. For example, for Alt-Random, random initialization was used with a small offset (\hat{λ}ₙ˜Uniform(0,1)+0.1) to avoid making any particular λₙ irretrievable from the start.

A few “guided” initialization processes were also explored. The quantity Σₙλ*ₙ can hypothetically be estimated from data if P(x_{d}=0|λ) is significant and easily estimated from Y. In fact, quantification in microfluidics often relies on a clear identification of empty sample partitions (that is, where x_{d}=0) (Basu, 2017). This motivates a strategy of relaxing the problem by optimizing X with a Poisson assumption on the sum of each column Σₙx_{n,d} rather than each element of X individually. The Σₙλₙ* Oracle is given Σₙλₙ* and optimizes for P(Σₙx_{n,d}|Σₙλₙ*) in place of P(x_{d}|λ) in (20). It is straightforward to show that this objective is also concave. Each estimate R d is solved via BB from which

$$\hat{λ} = \frac{1}{D} {\sum }_{d} {\hat{x}}_{d} .$$

The inventors use the Σₙλₙ*-Oracle as its own baseline and as an initialization to the inventors' alternating framework (Alt-Σₙλₙ*). For the next guided initialization, Σₙλₙ* was again used for an unbiased initialization where the first estimate of \hat{λ}ₙ=(Σ_{n′}λ_{n′}*)/N for all n (Alt-Unbiased). Finally, the output of SPoRe was used as an initial value for \hat{λ} (Alt-SPoRe). Alt-SPoRe can be understood as a way to use SPoRe to estimate \hat{X} if needed.

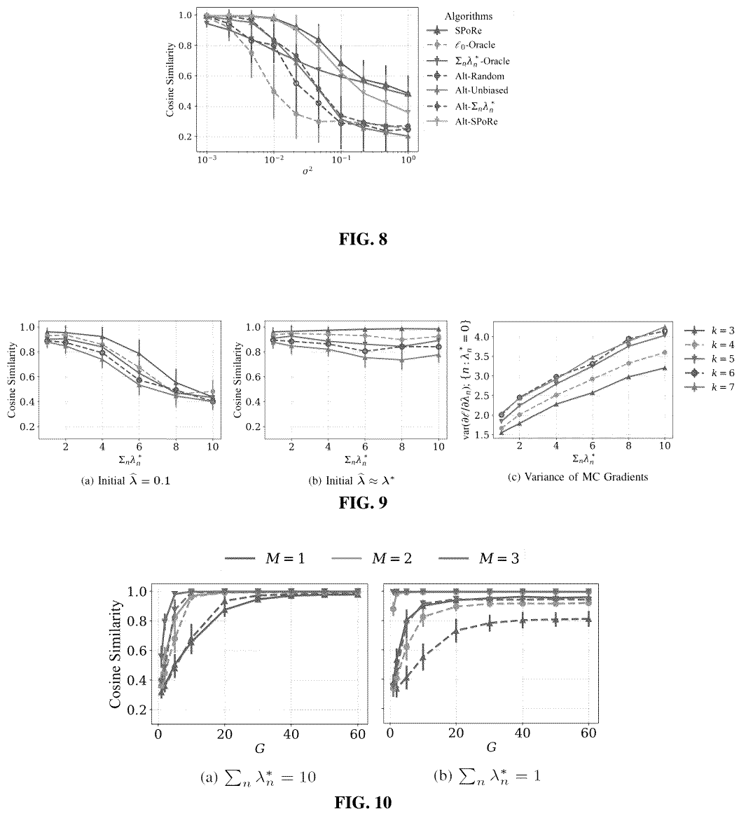

FIG. 8 illustrates that SPoRe has the greatest tolerance to measurement noise whereas the [symbol] ₀-Oracle has the least. This comparison illustrates the value of incorporating the Poisson assumption in recovery; specifically, the integer and sparsity structures (perfectly captured by the [symbol] ₀-Oracle) are not sufficient for recovery under measurement noise. The alternating optimization algorithm's behavior was unexpected; initialization (other than with Alt-SPoRe) does not appear to have a major influence on performance. Surprisingly, comparing Alt-Σₙλₙ* versus Σₙλₙ*-Oracle and Alt-SPoRe versus SPoRe, alternating seems to worsen the performance under high noise. The interpretation was that in high noise settings, the ability of SPoRe to not “overcommit” to a particular solution \hat{x}_{d} may be especially effective when λ* is the signal of interest rather than X*. Any given measurement y_{d} may make the specific estimate \hat{x}_{d} arbitrarily unreliable. In the alternating framework with \hat{x}_{d} recovered separately for each d, errors on individual estimates accumulate. SPoRe instead makes gradient steps based on batches of observations, helping it maintain awareness of the full distribution of measurements.

Sparsity and Σₙλₙ*. The limitations of SPoRe's recovery performance were empirically tested under very challenging conditions of M=2, 3≤k≤7, N=50, σ²=10⁻². Here D=1000 was set to better reflect the typical capabilities of biomedical systems, whereas D=100 in the baseline comparisons was due to the inventors' budget on computational time strained by solving BB for \hat{x}_{d}. From the analysis and previous simulations, it was expected that both k and the magnitudes of λₙ* will influence recovery. FIG. 9 probes when and why SPoRe fails. FIG. 9A illustrates SPoRe's performance decreases with increasing k and Σₙλₙ*. To elucidate the cause of poor performance, FIG. 9B shows SPoRe's performance under the same conditions when initialized at the optimum. SPoRe's maintenance of high cosine similarity in this case means that in FIG. 9A, SPoRe is converging to incorrect optima (or terminating before convergence). These two figures depict fundamental limitations of stochastic optimization in a challenging landscape.

Moreover, FIG. 9C illustrates that MC gradients decrease in quality with high Σₙλₙ* and k. In SPoRe, recall that was set at a minimum \hat{λ}ₙ=10⁻³ so that nonzero x* have a chance of being sampled for all n. S was kept fixed as k was increased and Σₙλₙ*, and it was seen that the variance of the gradient increased at coordinates where \hat{λ}ₙ=ϵ and λₙ*=0. Such an effect accounts for some drift from the optimum observed in FIG. 9B that increases with k, and it was believed that it helps to explain the inability to converge to the optimum in FIG. 9B. Alternative techniques may be used for stochastic optimization and sampling. Practitioners may benefit significantly from reducing Σₙλₙ* if faced with limitations in M. In a biosensing context, these results indicate that given a microfluidics system with fixed D, having fewer total analytes in the sample (i.e., smallerD Σₙλₙ*) can counterintuitively improve performance and may be particularly important if both M and G are limited.

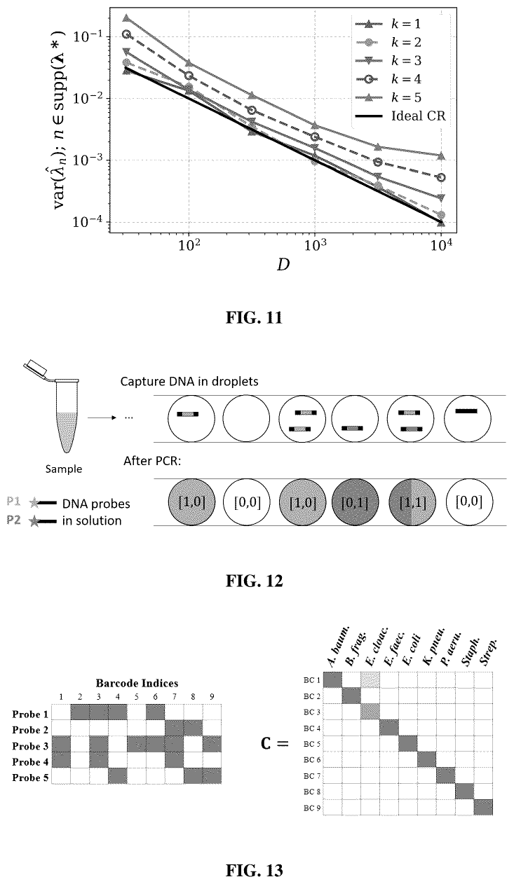

However, note in (8) that SPoRe's gradients are defined by an average of xₛ weighted by p(y_{d}|xₛ). The previous result from FIG. 7C in which SPoRe performed well with Σₙλₙ*≤20 when M=10 illustrates that limitations of MC sampling may be offset by improving the ability of p(y|xₛ) to guide gradients. In FIG. 10, this notion was explored further for M-constrained systems by increasing G. One may wonder how increasing G compares to a CS problem with GM measurements (i.e., Φ̅∈ [symbol] ^{GM×N}). Although λ* is fixed across groups, the x_{d} are random such that there is no reasonable method to directly stack individual measurements y_{d} from multiple groups. Instead, a new baseline [symbol] ₁-Oracle GM SMV was created. Denote the average of measurements and signals in each group g as y̅^{(g)} and x̅^{(g)}, respectively. The new baseline stacks all y̅^{(g)} into one measurement vector y̅^{(g)}∈ [symbol] ^{GM} and minimizes ∥y̅−Φ̅λ∥₂ with respect to λ given Σ_{n,d}x_{n,d}* and λ≥0. Stacking measurements and sensing matrices implicitly assumes that for each group, y^{-(g)}≈Φ^{(g)}λ*, or that x̅^{(g)}≈λ*∀g. It can be easily shown that the relative errors in these approximations reduce with increasing D or λ*.

Although increasing D is feasible in microfluidics, it generally corresponds with a reduction in λ* since a sample's total analyte content is fixed. Therefore, in FIG. 10, the influence was focused on of the magnitude of λ*. In FIG. 10A, the most challenging settings from FIG. 9 were used with k=7 and Σₙλₙ*=10. As expected from the above analysis, larger choices of M make SPoRe much more effective per sensor group, but near perfect recovery is achievable even with M=1. However, note that the new oracle baseline performs almost identically to SPoRe, with SPoRe exhibiting a modest advantage only when GM is comparable to or less than k. When Σₙλₙ* was reduced to 1 in FIG. 10B, the assumption that x̅^{(g)}≈λ* becomes far less valid. As a result, the performance improvement with SPoRe is dramatic. For instance, what SPoRe achieves with M=1 is only matched by the oracle baseline when M=3. For applications in which x̅^{(g)}* and GM>k, practitioners could consider reformulating the recovery problem as a standard CS problem. However, SPoRe is uniquely suited for systems with GM<k and is the best generalized approach for applications with smaller λ* or D.

Efficiency. For system design, it is helpful to know how many observations D are necessary and sufficient for stable estimation of \hat{λ}. Such insight is often derived from the analysis of Fisher Information [symbol] . Recall that for direct observations of Poisson signals x_{d}*, the ideal case, the MLE solution \hat{λ}ₙ=Σ_{d}x*_{n,d}/D is an efficient estimator and achieves the Cramer-Rao′ bound such that var(\hat{λ}ₙ)=λₙ*/D.

The reduction in [symbol] _{n,n} was derived from MMVP measurements and reason was found to expect that the reduction increases with k. Here, this effect was empirically characterized (FIG. 11). The matrix [symbol] was evaluated at λ*, so only n∈supp(λ*) was considered. In MMVP, the variance of \hat{λ}ₙ will depend on Φ and n, but by redrawing random Φ and λ* over 50 trials, it was hoped to smooth out these dependencies and capture the broader effect of low dimensional projections. For a concise comparison considering n∈supp(λ*), all λₙ*=1 were set, pooled all of \hat{λ}ₙ across all trials, and computed a single average variance for each k and D. Because the Fisher Information describes the optimization landscape near the optimum, parameter settings (M=2, G=20) were chosen based on the results in FIG. 10A to be confident that SPoRe is arriving near the optimum and the estimation variance is not confounded by poor estimates.

FIG. 11 shows a noticeable increase in the estimation variance and verifies that this deviation from the ideal bound is exponentially worsened in k. However, it was argued that the variance quickly becomes negligible at reasonable D for practical purposes. Practitioners could consider the necessary precision of estimation and the maximum expected k for an application, increase D as needed, and worry little about the influence of noisy measurements in low dimensions.

It was found that the structure in the MMVP problem can be easily exploited for substantial improvements in signal recovery. While compressed sensing of arbitrary integer signals has proven challenging in the past, Poisson constraints not only make the recovery problem tractable, but even significantly easier. Most inverse problems necessitate constraints that make the signal-to-measurement transformations nearly isometric: in compressed sensing, these manifest as restrictions on Φ, noise, and the relationship between M, N, and k. In MMVP, recovery of λ* is theoretically possible under very lax conditions on Φ (Theorem III.2) and practically achievable as shown in the inventors' simulations.

In practice, the new SPoRe algorithm exhibits high performance even under deliberately challenging circumstances of high noise and M<k. Here, recovery with G=1 appears particularly feasible even with noisy measurements if Σₙλₙ* is small. Because the log-likelihood is not concave, SPoRe's gradient ascent approach is not theoretically guaranteed to find a global optimum since local optima may exist. However, if they exist, it was speculated that SPoRe is naturally poised to evade these traps due to stochasticity in its gradient steps from both batch draws and MC integrations.

A few scenarios were noted in which SPoRe's MC sampling appears to cause issues with convergence or early termination that are generally associated with increases in k and Σₙλₙ*. It was anticipated that further increases in N may also contribute to these effects. While k and N are entirely determined by the application, system designers can reduce Σₙλₙ* by increasing the spatial or temporal sampling rate. In microfluidics, this adjustment translates to either generating more (smaller) partitions D given a fixed sample volume or diluting a sample prior to partitioning. The initial implementation of SPoRe uses S=1000, can easily run on personal computers, and is sufficient for systems with N<10 (The full code base is available at github.com/pavankkota/SPoRe with instructions on how to implement SPoRe with custom models). This scale is appropriate for most applications in biosensing, and future work with parallelized or adaptive sampling strategies could improve the reliability of recovery for larger systems. Moreover, it was found that increasing M and G appear to mitigate poor performance due to excessive sampling noise.

To date, CS implementations with low M and high G, based on the inventors' notation, reformulate the acquired measurements into a canonical CS problem with GM measurements (Duarte et al., 2008). As discussed above, this reformulation implicitly assumes that the average signal in every group (x̅^{(g)}) is approximately λ*. However, this assumption fails in biosensing in two common scenarios: the total number of analytes in a typical sample may be small, or the sensing mechanism may impose restrictions on D. In such cases, SPoRe is the superior approach allowing M<k, achieving high performance even with M=1 under high noise. In this work, practical limits on the sparsity level k were found that could accurately be solved, hence the “S” in SPoRe. High performance in this regime may be used in biosensing for the analysis of heterogeneous samples with many types of cells.

The ability to recover signals in MMVP, even in the extreme case of M=G=1, is unprecedented in CS and offers a new paradigm for sensor-constrained applications. The current state-of-the-art method for achieving similar efficiency in microfluidics is to essentially guarantee single-analyte capture for classification by substantially increasing the sampling rate, whereas the MMVP framework is not reliant on such an intervention. Increasing G can make SPoRe reliable under harsh conditions, is straightforward with microfluidics, and offers a potent alternative to increasing the sampling rate. For example, diluting to a tolerable concentration is challenging with samples of unknown content such as in diagnostics applications, and increasing D either delays results or necessitates high throughput measurement acquisition which may not be feasible depending on the nature of the sensors.

The theoretical and empirical results showed the promise of MMVP, but there are many directions for further research. For instance, theoretical results that precisely relate M, N, D, k, λ* and noise such as in a recovery guarantee would be highly valuable. Moreover, SPoRe can accept any signal-to-measurement model p(y|x). While the inventors have proven identifiability under linear mappings with common noise models, SPoRe can be applied with any application-specific model even if proving identifiability of the Poisson mixture is difficult.

### APPENDIX

A. Proof of Theorem III.2

A direct proof was used, assuming P( [symbol] ᵤ|λ)=P( [symbol] ᵤ|λ′) ∀u and proving the resulting implication λ=λ′. Let z^{(x)}∈ [symbol] be such that x+z^{(x)}∈ [symbol] ₓ. By Definition III.4, x+z^{(x)}∈ [symbol] ₊^{N} and z^{(x)}∈ [symbol] (Φ).

Lemma A.1. If [symbol] (Φ)∩ [symbol] ₊^{N}={0}, and P( [symbol] ₀|λ)=P( [symbol] ₀|λ′), then Σₙλₙ=Σₙλₙ.

Proof. The null space condition on Φ means that [symbol] ₀={0}; there is no vector z⁽⁰⁾ that satisfies 0+z⁽⁰ ∈ [symbol] ₊^{N} other than z⁽⁰⁾=0. Therefore, P( [symbol] ₀|λ)=P( [symbol] ₀|λ′)⇒e^{-Σ}^{ₙ}^{λ}^{ₙ}e^{-Σnλ′n}⇒Σₙλₙ=Σₙλ_{′n}.

Attention was turned to the one-hot collision sets. Let eⱼ denote the jth standard basis vector. By Definition III.4,

[symbol] ₑ_{ⱼ}={x:Φx=ϕⱼ, x∈ [symbol] ₊^{N}}. For [symbol] ₑ_{ⱼ} that contain only eⱼ, the inventors have the following result:

Lemma A.2. If [symbol] (Φ)∩ [symbol] ₊^{N}={0}, and [symbol] ₑ_{ⱼ}={eⱼ}, then λⱼ=λⱼ′.

Proof. The restriction on [symbol] ₑⱼ means P( [symbol] ej|λ)=P( [symbol] ej|λ′)⇒λⱼe^{-Σ}^{ₙ}^{λ}^{ₙ}=λⱼ′e^{-Σ}^{ₙ}^{λ}^{ₙ}. Applying Lemma A.1 yields λⱼ=λⱼ′.

By similar arguments to Lemmas A.1 and A.2, Corollary III.3 was proven under the assumption that there are no collisions instead of the null space condition. When the elements of Φ are independently drawn from continuous distributions, the collision of any particular x and x′ occurs with probability zero. Since [symbol] ₊^{N}× [symbol] ₊^{N} is countably infinite, there are no collisions almost surely. As such, [symbol] ₑ_{ⱼ}={eⱼ} ∀j, and therefore λ=λ′.

For the conditions in Theorem III.2, the following Lemma states the existence of at least one j satisfying [symbol] ₑ_{ⱼ}={eⱼ}:

Lemma A.3. If [symbol] (Φ)∩ [symbol] ₊^{N}={0} and ϕₙ≠ϕ_{n′}∀n,n′∈{1, . . . , N} with n/=n′, then ∃j such that [symbol] ₑ_{ⱼ}={eⱼ}.

Proof. If [symbol] ₑ_{ⱼ}={eⱼ}, then [symbol] z^{(e}^{ⱼ}⁾≠0. Define P as the number of one-hot collision sets that contain more than just eⱼ, and note that P≤N. Without loss of generality, let one say that [symbol] ₑ_{ⱼ} for j∈{1, . . . , P} meet this condition. Lemma A.3 effectively says that P<N, such that N−P>0 one-hot collision sets contain only eⱼ. A proof was then proceeded with by contradiction by assuming P=N.

By the null space condition, z^{(e}^{ⱼ}⁾ must contain both positive and negative integers. There are two additional conditions on nontrivial z^{(e}^{ⱼ}⁾. First, because eⱼ+z^{(e}^{ⱼ}⁾∈ [symbol] ₊^{N}, the only negative component of z^{(e}^{ⱼ}⁾ is zⱼ^{(e}^{ⱼ}⁾=−1. To see this, if zᵢ^{(e}^{ⱼ}⁾ for i≠j were negative, then eⱼ+z^{(e}^{ⱼ}⁾ would be negative at index i, and if zⱼ^{(e}^{ⱼ}⁾ were less than −1, then eⱼ+z^{(e}^{ⱼ}⁾ would be negative at index j. Second, z^{(e}^{ⱼ}⁾'s positive elements must total to at least 2. A single positive element of zᵢ^{(e}^{ⱼ}⁾=1 would imply that ϕᵢ=ϕⱼ, violating a condition on Φ.

With P=N, let one concatenate the z^{(e}^{ⱼ}⁾ column vectors into a matrix for visualization.

$$Z = [ \begin{matrix}- 1 & {z}_{1}^{( {e}_{2} )} & … & {z}_{1}^{( {e}_{N} )} \\ {z}_{2}^{( {e}_{1} )} & - 1 & … & {z}_{2}^{( {e}_{N} )} \\ ⋮ & ⋮ & ⋱ & ⋮ \\ {z}_{N}^{( {e}_{1} )} & {z}_{N}^{( {e}_{2} )} & … & - 1\end{matrix} ] . \qquad ( 22 )$$

Note that each column z^{(e}^{ⱼ}⁾ in this matrix is symbolic for any vector that satisfies the conditions the inventors described. All columns of Z are in [symbol] (Φ). Any linear combination of vectors in [symbol] (Φ) are in [symbol] (Φ). Let [symbol] represent a subset of indices of the columns of Z and let z^{S} [symbol] Σ_{j∈S}z^{(e}^{ⱼ}⁾.

First, let [symbol] {1, . . . , N}. Because all off-diagonal components in Z are nonnegative and because z [symbol] must have one negative value, one of the rows of Z must be entirely zero except for the −1 on the diagonal. Note the ordering of the columns in Z is arbitrary, so without loss of generality, let this be the first row. Now, let's say that [symbol] ={2,3, . . . , N}. The same logic holds: at least one row must contain all zeros except for the −1. Without loss of generality, the inventors can set [Z]_{2,3}, [Z]_{2,4}, . . . [Z]_{2,N}=0. Repeating this process, they get a lower triangular matrix:

$$Z = [ \begin{matrix}- 1 & 0 & 0 & … & 0 \\ {z}_{2}^{( {e}_{1} )} & - 1 & 0 & … & 0 \\ {z}_{3}^{( {e}_{1} )} & {z}_{3}^{( {e}_{2} )} & - 1 & … & 0 \\ ⋮ & ⋮ & ⋮ & ⋱ & ⋮ \\ {z}_{N}^{( {e}_{1} )} & {z}_{N}^{( {e}_{2} )} & {z}_{N}^{( {e}_{3} )} & … & - 1\end{matrix} ] . \qquad ( 23 )$$

However, examining the final column, it was seen that z^{(e}^{_{N}}⁾ is a vector of all zeros and one −1, such that it cannot be in [symbol] (Φ), proving Lemma A.3 by contradiction.

Proof of Theorem III.2: Lemma A.3 confirms P<N, meaning that the inventors can form the concatenated matrix of z^{e}^{ⱼ} vectors:

$$Z = [ \begin{matrix}- 1 & 0 & … & 0 \\ {z}_{2}^{( {e}_{1} )} & - 1 & … & 0 \\ ⋮ & ⋮ & ⋱ & ⋮ \\ {z}_{P}^{( {e}_{1} )} & {z}_{P}^{( {e}_{2} )} & … & - 1 \\ ⋮ & ⋮ & ⋱ & ⋮ \\ {z}_{N}^{( {e}_{1} )} & {z}_{N}^{( {e}_{2} )} & … & {z}_{N}^{( {e}_{P} )}\end{matrix} ] . \qquad ( 24 )$$

Let P(Cₑ_{_{P}}|λ)=P(Cₑ_{_{P}} ═λ′) be applied. For all x∈Cₑ_{_{P}},

$${\prod }_{n = 1}^{N} \frac{{λ}_{n}^{{x}_{n}}}{{x}_{n} !} - {\prod }_{n = 1}^{N} \frac{{λ}_{n}^{′ {x}_{n}}}{{x}_{n} !} \vert = 0 , \qquad ( 25 )$$

$$( {\prod }_{∀ i > P} \frac{{λ}_{i}^{{x}_{i}}}{{x}_{i} !} ) ( \frac{{λ}_{P}^{{x}_{P}}}{{x}_{P} !} - \frac{{λ}_{P}^{′ {x}_{P}}}{\vert {x}_{P} !} ) = 0. \qquad ( 26 )$$

where Lemma A.1 (Σₙλₙ=Σₙλ′ₙ) yields the first equality, and Lemma A.2 (all λᵢ=λ′ᵢ∀i>P) yields the second equality when combined with the fact that xᵢ=0 for i<P due to (24). The only x∈ [symbol] ₑ_{_{P}} with x_{P}≠0 is e_{P} which simplifies (26) to λ_{P}=λ′_{P}.

Now there is λᵢ=λ′ᵢ∀i>P−1. Following the same arguments, P( [symbol] ₑ_{_{P-1}}|λ)−P( [symbol] ₑ_{_{P-1}}|λ′) can be started from and arrived at λ_{P-1}=λ′_{P-1}. Applying this repeatedly ultimately yields λ=λ′, proving Theorem III.2.

Proof of Proposition III.5

Proof. By straightforward integration,

$${E}_{y \vert {n}^{*}} [ p ( y \vert n ) ] ∝ {\underbrace{exp \{ - \frac{1}{4 {σ}^{2}} {‖ {ϕ}_{n} - {ϕ}_{{n}^{*}} ‖}_{2}^{2} \}}}_{{=}^{△} κ ( n , {n}^{*} )} . \qquad ( 27 )$$

Therefore, given the constraints ∥λ∥₁≤1 and λₙ≥0, the first-order KKT condition is

$${𝔼}_{{n}^{*}} [ \frac{κ ( {n}^{*} )}{( κ ( {n}^{*} ) , λ )} ] = cs - μ , \qquad ( 28 )$$

where κ(n*)=(κ(n,n*))ₙ₌₀^{N}, s∈∂∥λ∥₁, c≥0, and λₙ≥0. By complementary slackness, λₙλₙ=0 for all n. Because K is symmetric, the above was rewritten as

$$K ( \frac{{λ}^{*}}{K λ} ) = cs - μ , \qquad ( 29 )$$

where the fraction represents element-wise division. Solving for λ and rescaling μ, the desired expression was obtained.

Proof of Theorem III.6

Proof. Let κ_{g} be defined the same as κ from (27) with ϕₙ^{(g)}. Then again by straightforward integration,

$$R ( n , {n}^{*} ) {=}^{△} {𝔼}_{g} [ {κ}_{g} ( n , {n}^{*} ) ] \qquad ( 30 )$$

$$= \{ \begin{matrix}1 & n = {n}^{*} , \\ {( \frac{2 {σ}^{2}}{2 {σ}^{2} + 1} )}^{M / 2} & n \neq {n}^{*} , 0 ∈ \{ n , {n}^{*} \} , \\ {( \frac{{σ}^{2}}{{σ}^{2} + 1} )}^{M / 2} & n \neq {n}^{*} , 0 ∉ \{ n , {n}^{*} \}\end{matrix} . \qquad ( 31 )$$

Let

$$\tilde{K} = {( \tilde{κ} ( n , {n}^{′} ) )}_{n , {n}^{′} = 0}^{N} ,$$

and let

$$\hat{K} = {( \tilde{κ} ( n , {n}^{′} ) )}_{n , {n}^{′} = 1}^{N}$$

be the sub-matrix of \tilde{K} excluding n=0. Then

where

$$a = {( \frac{{σ}^{2}}{{σ}^{2} + 1} )}^{M / 2}$$

and J is a matrix of all ones. Leveraging the block matrix inverse, it was observed that there was the form

$${\tilde{K}}^{- 1} = [ \begin{matrix}\frac{1}{1 - a} I + bJ & c 1 \\ c {1}^{T} & d\end{matrix} ] . \qquad ( 33 )$$

assuming the final column corresponds to n=0, for some scalars b, c, and d. Using the formula from Proposition III.5 and the fact that s−μ=1 by assumption, it was conclude that for n>0, \tilde{λ}ₙ∝λₙ*+C for some C. Rote algebra verifies that the constant of proportionality is non-negative.

### Example 2—Scalable Diagnostics with Microfluidics

The inventors recently coupled advances in microfluidic partitioning with ideas from compressed sensing to address these challenges. Compressed sensing (CS) seeks to infer sparse signals efficiently: faster or with fewer sensors (Baraniuk, 2007; Donoho, 2006). In biosensing, samples are sparse when among many possible analytes, only a handful are present in any given sample. For instance, a patient could be infected with any of hundreds of pathogens, but only one or a few are responsible for the current infection (Peters et al., 2012). In this application, CS is analogous to quantifying analyte fingerprints from mixed measurements (Aghazadeh et al., 2016). The inventors recently developed new theory and a new Sparse Poisson Recovery (SPoRe) algorithm that couples principles of CS with microfluidic partitioning (Kota et al., 2022). SPoRe performs maximum likelihood estimation (MLE) with gradient ascent over a generalized likelihood function. While the inventors chose to explore this direction because microfluidics approaches the limit of sensitivity through single-molecule analysis, they also found fundamental advantages from a signal processing perspective. Most notably, leveraging the Poisson-distributed capture of analytes enables improved rates of multiplexing (fewer sensors, more analytes), tolerates multi-analyte capture in the same partition, withstands very high measurement noise, and can enable partial fingerprints to be captured separately in sensor-constrained systems. The inventors call this latter concept asynchronous fingerprinting.

The inventors' initial research focused on linear measurements to contextualize their research in the field of compressed sensing, but the inventors found that SPoRe can be readily applied to any sensing model. In this work, the inventors first derive broader sufficient conditions for the types of practical sensors that can apply the inventors' framework. Next, they present the first in vitro demonstration of the inventors' framework towards bacterial infection diagnostics, quantifying nine pathogen genera with only five orthogonal DNA probes in droplet digital PCR (ddPCR). The five probes assign binary barcodes to the 16S rRNA genes of these bacteria, functioning as the fingerprints in the inventors' system. However, given a limitation of only two fluorescence channels on their system, the inventors apply the concept of asynchronous fingerprinting. They split a sample into four groups of droplets, each with a subset of two probes, and the inventors infer the concentrations of the bacteria with SPoRe over the pooled dataset from all droplet groups. They characterize the performance of the inventors' assay with 18 samples each with a mixture of 2-4 bacteria and extend the inventors' characterization in simulated experiments. Finally, they show how their probabilistic framework enables the flagging of samples that contain 16S barcodes outside of the designed panel.

In ddPCR, microfluidics splits a sample into thousands of droplets to stochastically capture nucleic acids (FIG. 12). Endpoint PCR measurements form binary clusters that indicate the presence or absence of target sequences (Quan et al., 2018; Whale et al., 2016). The inventors used the Bio-Rad Qx 200 (Bio-Rad Laboratories, Hercules, CA, U.S.A.) which has two fluorescence channels (FAM and HEX) for multiplexed PCR with hydrolysis probes.

The inventors designed nonspecific 11-nucleotide hydrolysis probes spiked with locked nucleic acids (LNAs) that bind to the 16S genes of a panel of 12 bacterial species. Briefly, the inventors filter the set of 11-mers to avoid heterodimers and weak mismatches while ensuring a sufficiently high melting temperature. Each 16S gene elicits a binary barcode response to the set of five candidate probes based on the presence or absence of the probe sequences in the gene (FIG. 14A). The inventors used coordinate ascent optimization to select a final probe set that separated the bacteria by genus. Particularly, the inventors group three species of Staphylococcus together (S. aureus, S. epidermidis, and S. saprophyticus) and two species of Streptococcus (S. agalactiae and S. pneumoniae).

There are multiple 16S gene copies in each bacterial genome with slight sequence variability. Although the inventors attempted to design probes such that each genus had a unique, consistent barcode for all copies, E. cloacae appeared to exhibit a small proportion of variant barcodes, a fraction which the inventors computed experimentally (Methods 4.8). Such variation is likely inevitable especially in larger scale systems but can be accounted for. The inventors store each pathogen's fractional barcode distribution across its copies in a column of a matrix C (FIG. 13). Note the ordering of barcodes is arbitrary in constructing C and that because of only slight variation between 16S copies within a genome (Johnson et al., 2019), C is nearly the identity matrix in practice. In the rows of C, the inventors also ignore the barcodes that are not elicited by the combination of probes and the bacterial panel. The inventors' optimization estimates the 9-dimensional Poisson parameter vector λ that represents 9 analyte concentrations. With 9 unique barcodes and bacterial genera, the analytes could refer to either. If they are the barcodes, then λₙ^{(BC)} is the concentration of the total 16S genes from any source bacteria that exhibits the nth barcode.

If the analytes are the bacteria, then λₙ^{(bact)} is the concentration of the nth bacteria's 16S genes, regardless of the particular barcodes of individual genes. Because λₙ^{(BC)}=Cλₙ^{(bact)}, these results currently depend on a C matrix of rank N to readily convert between the barcode concentrations λ^{(BC)} and the bacterial concentrations λ^{(bact)}. For context-dependent reasons, the inventors make use of both definitions of “analyte” and carefully clarify which they are using at any time, and they often drop the superscript notation.

Ideally, the inventors could estimate λ^{(BC)} by simply capturing individual 16S genes in droplets with all five probes (Section 5.1). However, the unique clusters only hold for single-gene capture; if multiple genes appear (with distinct sequences) in the same droplet, an effect known as partition specific competition (PSC) occurs and fluorescent intensities can decrease (Whale et al., 2016). Second, unique clusters for every barcode cannot scale beyond this study if the eventual goal is to quantify dozens to hundreds of microbes.

Instead, the inventors generate four sensor groups of droplets each with a different subset of two probes (FIG. 14A). The inventors call this concept asynchronous fingerprinting and describe the allocation of probes to each group in the inventors' mathematical theory (Theorem 4.7 in Section 4.1.3). Despite competition effects, raw droplets can still be reasonably thresholded above zero in each channel (Whale et al., 2020). Although the 16S barcodes in each droplet is made entirely ambiguous, the inventors infer bacterial concentrations in the sample from the pooled, binarized data from these four groups of droplets. SPoRe essentially finds the solution that best explains the distribution of droplet measurements in the four groups.

Generalized Maximum Likelihood Estimation with Microfluidic Partitioning. The standard for quantification in digital microfluidics data is based on MLE (Majumdar et al., 2015). The inventors generalize the MLE approach and show how the conventional equations can be derived from special case assumptions (Section 5.3). Their particular approach with ddPCR is one realization of the inventors' generalized MLE framework. Respectively, the general terms used in this section analyte, partition, and measurement vector correspond with the physical concepts of a barcode or bacterium, a droplet, and the two pre-binarized measurements acquired from each droplet. Section 4.1.1 contains detailed clarification of the inventors' mathematical notation.

Let N and D define the number of unique analytes in the assay and the number of partitions, respectively. The inventors let x_{d} be an N-dimensional nonnegative integer vector representing the quantities of each analyte in partition d. The inventors say λ is k-sparse if k elements are nonzero. With microfluidic partitioning, x_{d} is distributed as Poisson(λ) where λ is the N-dimensional parameter vector that characterizes the rate of capture of each of N analytes. Let y_{d} represent the measurement vector acquired from partition d (e.g., in the inventors' assay, y_{d}∈{0,1}²). Note that while y_{d} is observed directly, λ must be inferred and x_{d} is latent. The inventors use an asterisk (λ*, x_{d}) to denote true values and a hat (\hat{λ}, x_{d}) to denote estimates. The goal in MLE is to find an estimate \hat{λ}_{MLE} that maximizes the likelihood of the observed measurements. A general form of the likelihood function is:

$${\hat{λ}}_{MLE} = {arg max}_{λ} {\prod }_{d = 1}^{D} p ( {y}_{d} \vert λ ) \qquad ( 1 )$$

$$= {arg max}_{λ} \frac{1}{D} {\sum }_{d = 1}^{D} log {\sum }_{x ∈ {ℤ}_{+}^{N}} p ( {y}_{d} \vert x ) P ( x \vert λ ) . \qquad ( 2 )$$

Denoting the likelihood function from the right-hand side of (2) as [symbol] , the gradient is

$${∇}_{λ} ℓ = \frac{1}{D} {\sum }_{d = 1}^{D} \frac{{\sum }_{x ∈ {ℤ}_{+}^{N}} p ( {y}_{d} \vert x ) P ( x \vert λ )}{λ {\sum }_{x ∈ {ℤ}_{+}^{N}} p ( {y}_{d} \vert x ) P ( x \vert λ )} - 1. \qquad ( 3 )$$

Although fairly obtuse, this equation leads to two commonly observed equations in specialized implementations of MLE that use digital fingerprinting or orthogonal assays (Section 5.3). As discussed, digital fingerprinting necessitates clear classification boundaries, and orthogonal assays in each partition restrict the number of analytes that can be practically quantified. In the inventors' ddPCR assay, no droplet can be assumed to have or lack a particular 16S barcode. Despite this ambiguity, the inventors' SPoRe algorithm uses gradient descent to iteratively solve Equation (2).

Modifications to SPoRe Algorithm. The inventors summarize two particular changes to the original SPoRe algorithm with implementation details left to Sections 4.7 and 5.2. First, they have noted the flexibility of SPoRe for any sensing model. This flexibility arises from the fact that p(y_{d}|x), the sensing model that maps signals to measurements, is the only term that must be defined for a particular application. The inventors use a simple model for the inventors' ddPCR assay. For y_{d}∈{0,1}² with M=2 for the two fluorescence channels, they say p(y|x)=Πₘp(yₘ|x) with p(yₘ|x)=1 if x has at least one copy of a gene that contains the corresponding probe, with p(yₘ|x)=0 otherwise. In the inventors' implementation, they define the analyte as the bacterial content and account for fractional barcode content in the gradient computations (Section 5.2).

Second, note in Equation (3) that the gradients contain an infinite summation over all of N-dimensional nonnegative integer vectors (Z₊^{N}). In the inventors' earlier work (Kota et al., 2022), the inventors used Monte Carlo approximations of this gradient on batches of measurements. They showed how these approximations became more variable with increases to k, and the inventors speculate the same would occur with increases to N. While the estimation variance can be reduced with more samples in the Monte Carlo approximation, this approach would increasingly rely on parallelized high performance computing as N or k increases. Fortunately, with the finite measurement space of ddPCR and the fact that binary probe measurements depend merely on the presence or absence of an amplicon with the complementary sequence, these gradients can be computed quickly and exactly over all measurements (Section 5.2). Given an exact gradient, the inventors use backtracking line search to speed up convergence of gradient descent to estimate λ_{MLE}^{(bact)}. Such accelerations are less feasible if the gradient is loosely approximated. The exact gradient, while cumbersome to derive for the 2-channel ddPCR system, could be similarly calculated for any number of channels and is computationally cheap.

Identifiability of Model. Running gradient descent to convergence will find a solution, but the inventors need to develop some assurance that it is the correct solution. Proving the identifiability of a model ensures that there is a unique global optimum to the likelihood function given infinite data. Formally, identifiability states that if p(y|λ)=p(y|λ′) for all y in each sensor group, then λ=λ′.

The inventors formally define terms and prove sufficiency conditions for identifiability in Methods 4.1. Briefly, they find that λ can be inferred uniquely if the sensing functions that dictate the mapping of x to y are monotonic and saturating. Monotonic functions do not change direction in output; in biosensing, most outputs increase with increases in input and vice versa such that the sensors are monotonic increasing. Any additional analyte should not decrease the output measurement. By saturating, the inventors mean that once a sensor has reached a saturating point in its response to an analyte, no further addition of that analyte will change the measurement value. Lastly, the inventors impose a system-wide condition called fingerprint equivalence which (informally) states that analytes with the same single-molecule fingerprints in a given sensing group behave interchangeably.

To align with these conditions, the inventors define the analytes as the barcodes. Under reasonable PSC effects (not an overwhelming diversity of initial 16S genies in a droplet) the addition of new barcodes to a droplet cannot reduce the binarized measurements (monotonicity). The binary data is determined by the presence of an 16S amplicon with a complementary probe sequence, with any quantity greater than zero resulting in the same digital signal (saturating). Lastly, gene copies with the same combination of probe binding sites are interchangeable (fingerprint equivalence). Therefore, Theorem 4.7 proves the conditions for λ^{(BC)}=λ′^{(BC)} but with rank(C)=N, λ^{(BC)}=λ′^{(BC)}⇒λ^{(bact)}=λ′^{(bact)}.

Here, the inventors provide an informal explanation and concrete example of the result of Theorem 4.7. This Theorem defines a matrix Z^{(g)} whose rows indicate the positions of analytes with equal, nonzero fingerprint responses in the sensor group g. Stacking these matrices for each g yields a matrix Z, and if rank(Z)=N, then the system is identifiable. FIG. 15 illustrates this process for the inventors' particular assay. With two binary measurements per group, there are 2² 1=3 nonzero barcode measurements. For instance, note that in Group 1, original barcode indices 2 and 4 share a [1,0] response, yielding the first row of Z). Each group contributes three rows to the matrix Z.

This result implies that the inventors cannot arbitrarily assign probes to each group, and interestingly, using probes in multiple groups can be beneficial by adding rows to Z. It is not sufficient to simply capture each probe's information in at least one group. Also, for rank(Z) to be N, the inventors can derive a simple rule of thumb for binary ddPCR with M channels: G(2^{M}−1)≥N is necessary for the conditions of Theorem 4.7. Although the inventors had access to two-channel Bio-Rad Qx200, this result also indicates promise for applying the inventors' framework to digital PCR systems with more than two channels: up to N=15G analytes such as on the 4-channel QxOne from Bio-Rad, or up to 63G analytes such as on a the six-channel naica system (Stilla Technologies, Villejuif, France). Note that the inventors do not intend to rank these instruments, as other factors such as the volume that can be processed, the number of partitions that can be generated, automation of workflows, etc. are also important factors that are application-dependent.

However, the implications of identifiability must be approached with caution. From an optimization perspective, identifiability simply implies that given infinite data (D→∞) in each group, the true λ* is the global optimum of the likelihood function. These results fall short of a recovery guarantee for a finite D partitions. In their earlier work (Kota et al., 2020), the inventors derived the insight that less sparse λ* (more analytes with nonzero quantities) necessitate more partitions for stable recovery. Nonetheless, in contrast to typical applications of CS, there is no explicit maximum for the sparsity level as any λ is identifiable under the conditions they derived.

Demonstration of Polymicrobial Diagnostics. The inventors tested SPoRe's ability to quantify bacterial loads in mixed samples of purified genomic DNA. They used reference wells with individual bacterial dilutions to estimate the ground truth concentration and assist with manual thresholding (Section 4.6) to binarize the data. The inventors passed this pre-binarized data to SPoRe.

FIG. 16 illustrates the quantitative results. For general performance evaluation on polymicrobial samples, the inventors use the cosine similarity metric to capture the inventors' framework's concordance with the true relative abundances of bacteria in the sample. They find an average cosine similarity of 0.96, indicating the inventors' ability to very reliably capture the dominant bacteria in a sample while making some errors on the relatively less abundant bacteria.

Solving an optimization problem can risk returning a locally optimal solution. However, the inventors find that the likelihood of the converged solution is higher than that of the supposed ground truth (FIG. 17), so it is unlikely that local optima explain the inventors' discrepancies.

Differing in concentration estimates for the bacteria truly in the sample is to be expected, as some of this error will arise simply from pipetting volume variablity and sampling variability in capturing 16S gene copies.

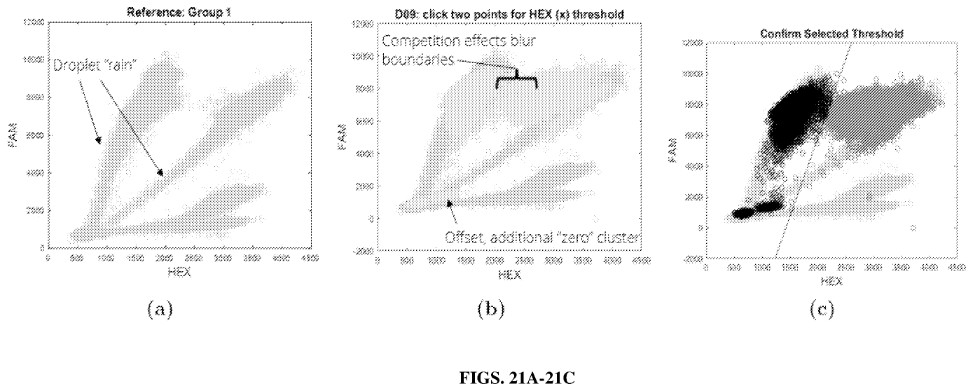

However, in some samples, SPoRe misses a bacteria of low abundance (a false negative) while including a bacteria that is absent in the sample (a false positive). The inventors hypothesize that these errors may be due to imperfect thresholding of the inventors' data that could easily be misclassifying some droplets. Because they pass pre-binarized data to SPoRe, mistakes in thresholding propagate to SPoRe; that is, given “warped” data, SPoRe will return a warped solution which could appear to have higher mean likelihood than the estimated ground truth (6). Some degree of droplet misclassification is very likely with the inventors' current assay for three reasons illustrated in FIG. 21. First, measurements currently exhibit very high droplet rain which the inventors suspect is due to the inventors' attempts to amplify a very long sequence: the 16S gene is roughly 1500 base pairs, while Bio-Rad recommends an amplicon of 75-200 base pairs.

Second, PSC appears to be blurring the boundaries between the typical binary clusters in droplets with more than one amplicon. Third, the negative droplets also exhibit some “lean” and “lift” most likely due to partial probe interactions with amplicons containing slightly mismatched sequences.

Recall that all 16S genes are expected to be exponentially amplified, and the probe signal the inventors receive is based on its interaction with the amplicons in each droplet. Overall, these combined effects squeeze the boundary in which they can draw thresholds. While these effects are common and have some popular tools to help disambiguate droplets (Jones et al., 2014, Brink et al., 2018), the inventors decided to use manual clustering as these tools are generally not designed for the conditions of the inventors' assay.

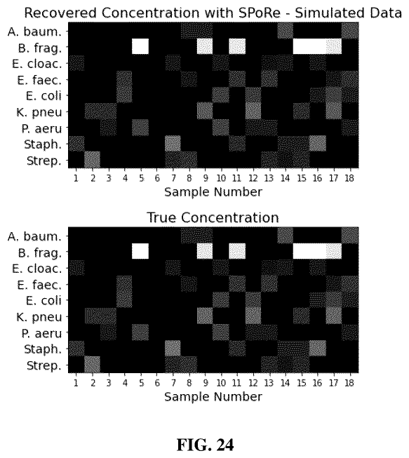

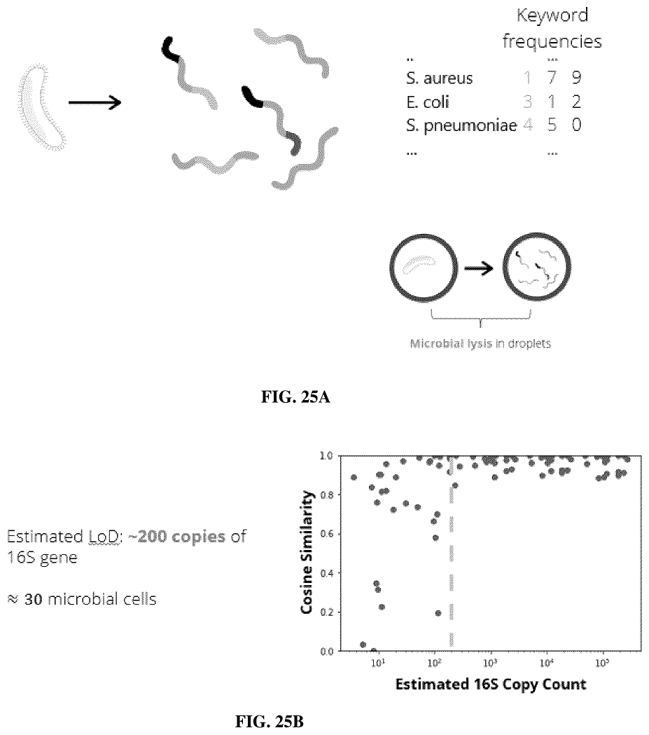

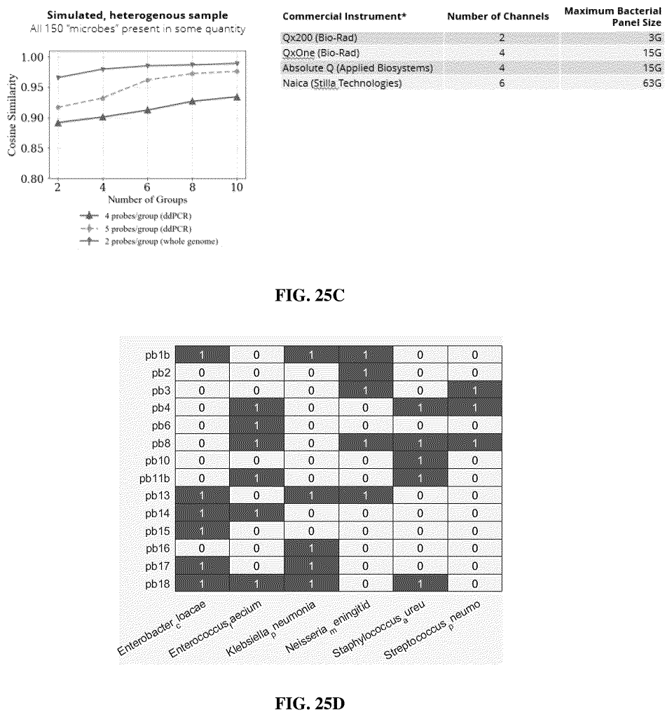

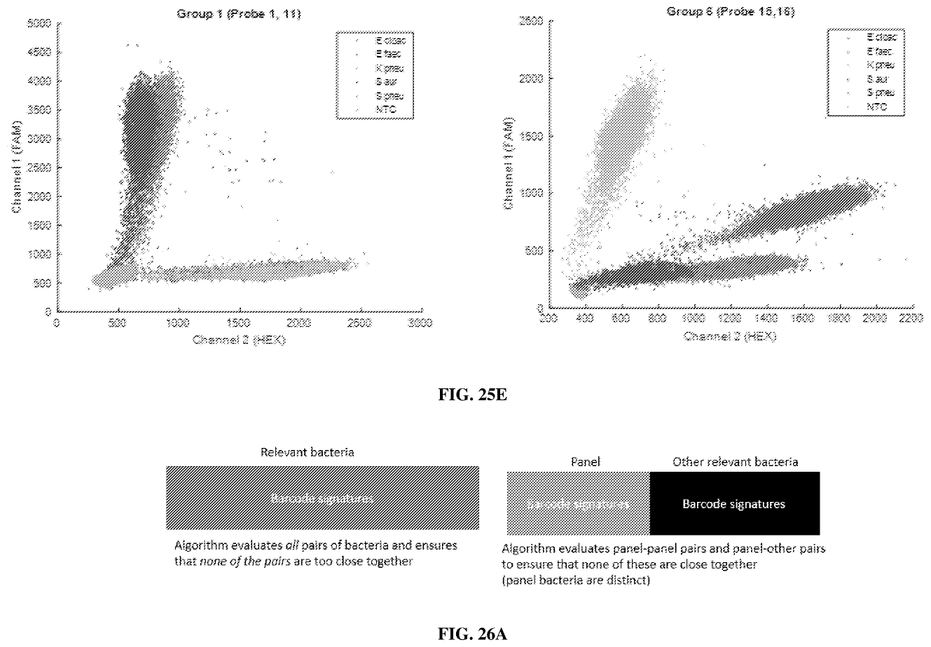

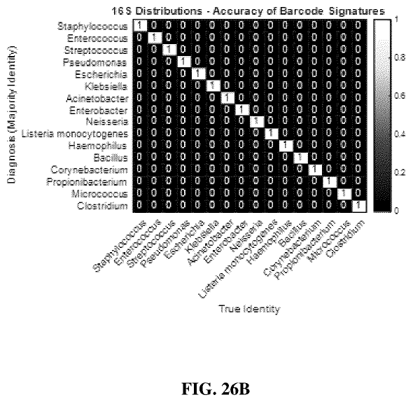

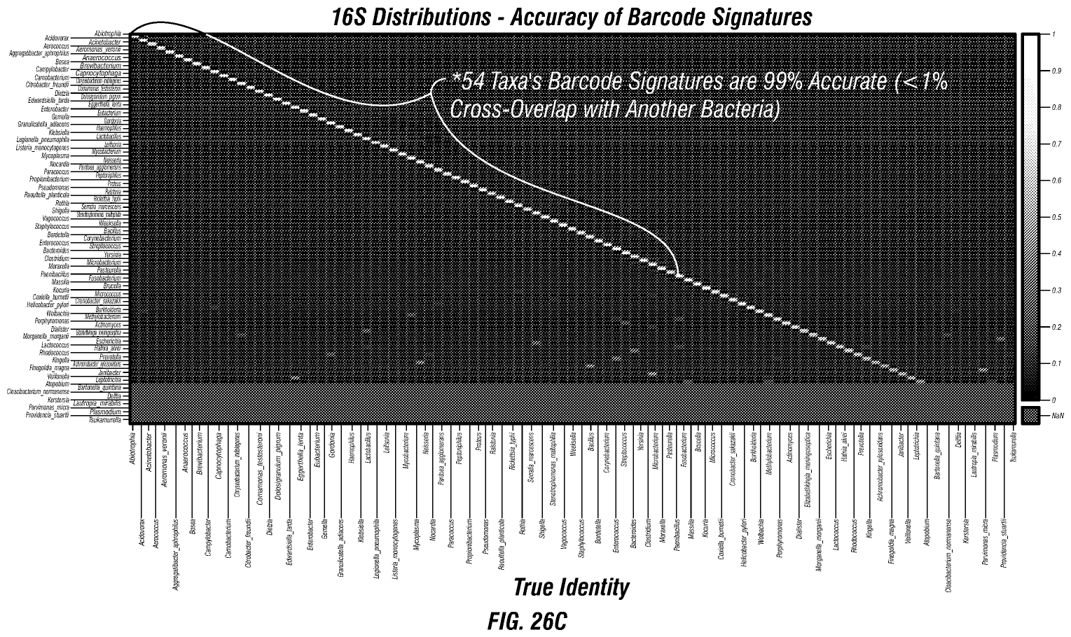

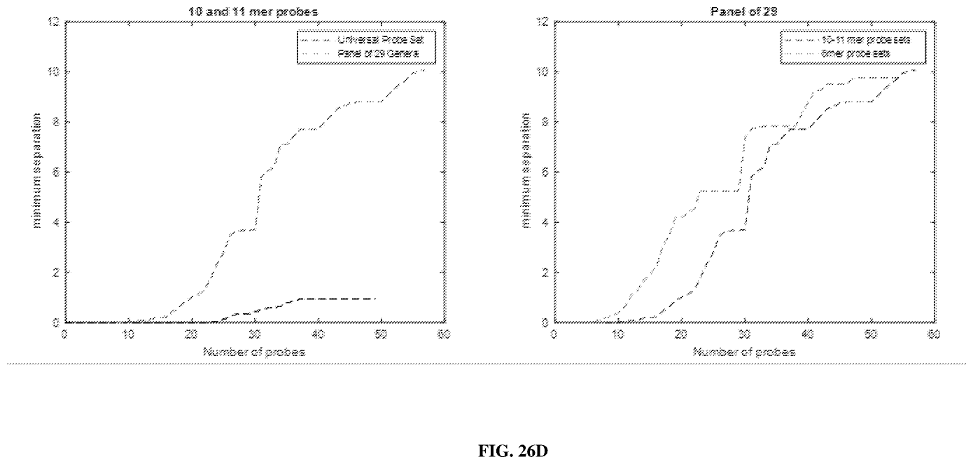

The inventors designed a simulated experiment to directly evaluate the effect of imperfect binarization of the raw measurements. Given the estimated ground truth concentrations and the droplet counts in each group, they simulated underlying droplet gene content (X with x_{d}˜Poisson(λ*)) and the resulting binary measurements using the inventors' p(y|x) model. On this simulated data, SPoRe returned virtually perfect solutions with a mean cosine similarity of 0.9997 (FIG. 24). This improved performance indicates that the most likely cause of slight errors with the inventors' thresholded data is due to the inventors' model's imperfect ability to represent the data. This discrepancy is natural given that the inventors' model asserts hard thresholds for binarization while the real data does not maintain strict decision boundaries. However, the inventors' high cosine similarity with the real data illustrates that the inventors' signal reconstruction is robust to some model imperfections in recovering the relatively high abundance bacteria in samples.

Characterization of Limit of Detection. In infection diagnostics, pathogen loads can vary by several orders of magnitude. Tolerating multi-gene capture reduces the risk that high concentration samples flood a system. The inventors designed samples such that total bacterial concentrations (Σₙλₙ*) would be between 1 and 5 to illustrate this ability. Tolerating multigene capture allows design flexibility for microfluidics systems with fewer partitions (e.g., smaller form factors with nanowells instead of droplets). However, demonstrating this capability on the Bio-Rad Qx200 meant that the inventors' samples have 16S concentrations that are unrealistically high for most clinical presentations.

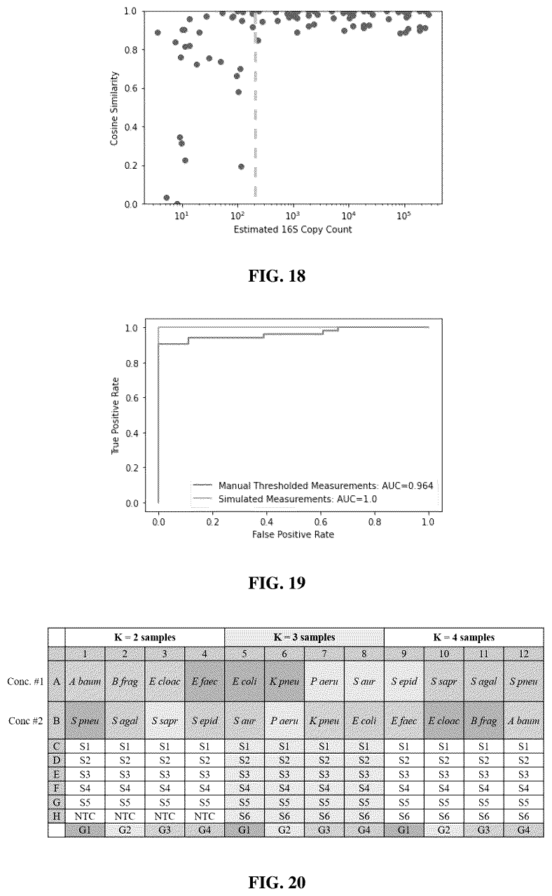

The inventors characterized the limit of detection in terms of 16S copy counts per sample for partitioning systems that may still result in multi-analyte capture by randomly subsampling the inventors' experimental data. For each sample, they subsampled 10%, 1%, 0.1% and 0.01% of the droplet data and passed it to SPoRe. The inventors estimate the 16S copy count in this data as the product of the number of subsampled droplets and the total estimated ground truth concentration Dₛ(Σₙλₙ*). FIG. 18 shows how SPoRe maintains strong recovery down to approximately 200 copies of the 16S gene.

Of course, a final system may have the flexibility to generate many partitions, meaning that at low 16S copy counts, non-empty partitions most likely have only one target molecule captured.

While this is uniquely unnecessary for the inventors' given fingerprinting approach, low magnitudes of λ empirically help recovery (Kota et al., 2022). Intuitively, if the same sample is split over more partitions, signal inference can only stand to gain information by capturing measurements from more individual molecules rather than their combined effects. Moreover, in ddPCR, single-molecule capture would avoid PSC altogether such that thresholding may be more reliable.

Flagging of Samples with Unknown Barcodes. A disadvantage of the inventors' system is that MLE will always report some solution even if the sample contains a bacteria with a different 16S barcode distribution outside of the panel given to SPoRe. However, the inventors can use the probabilistic nature to evaluate recovered solution in useful ways. They can characterize the expected distribution of the discrete measurement vectors given a recovered \hat{λ} and perform a χ² goodness of fit test between the expected and observed distribution of measurements.

For each tested polymicrobial sample, the inventors simulated the effect of having an “unknown” bacteria in it by removing each of the correct, present bacteria (one at a time) from SPoRe's database before running the algorithm. They repeated this process for both the manually thresholded and simulated data. In both cases, the p value of the test can be a highly reliable metric for flagging samples with out-of-panel bacteria as indicated by the receiver operating characteristic (ROC) curves (FIG. 19). With simulated data, the separation is perfect with an area under the curve (AUC) of 1.0. With manual thresholding, note that the pre-binarized data may yield an improbable distribution of measurements for any λ such that samples which contain only bacteria in the panel may nonetheless be flagged, leading to its diminished (but still strong) performance with an AUC of 0.964.

Reporting “unknown bacteria” is likely far more useful to a clinician than a “negative” result that would be returned from panels designed by specific sensors. This ability mirrors that of mass spectrometry and other fingerprinting systems, but the statistical underpinning could lead to theoretically grounded approaches with more research.

The inventors demonstrate a new scalable framework for infection diagnostics that leverages the sparsity of samples and the Poisson distribution of microfluidic capture. The inventors' nonspecific probes coupled with this framework enabled the quantification of 9 bacterial genera with 5 probes and 2 primers at levels as low as approximately 200 total copies of the 16S gene. The limit of detection appears to be sufficient for clinical applications, although the inventors' samples with only pre-extracted bacterial genomic DNA are highly idealized. Real-world samples with background nucleic acids are likely to hinder performance, but improvements in cluster separation through assay optimization may offset these effects.

More generally, the inventors present theoretical results that substantially broaden the types of sensors that allow λ to be identifiable. However, the inventors' SPoRe algorithm is modular for any user-defined sensing function, and the inventors' theoretical conditions are sufficient but not necessary. They encourage users to proceed with simulations even if their sensing model is outside the scope of the inventors' currently developed theory. As a nonconvex optimization process, the inventors' approach is not guaranteed to find the best solution. New theoretical results that provide convergence guarantees and worst-case error quantification would substantially increase user confidence in this approach. For the time being, the inventors have demonstrated a method for flagging solutions that are inconsistent with the observed data. This technique can help identify samples that contain analytes outside of the designed panel and solutions that have converged to poor local optima (if any). However, both the reliability of such flagging and the accuracy of estimated analyte quantities may be jeopardized if the model provided to SPoRe does not closely match real-world conditions.

The application of MLE to ddPCR data is not new, but the inventors' generalization offers unique insights and practical advantages for microfluidics-based diagnostics. These advantages stem from the inventors' tolerance of ambiguity from individual partition measurements.

Conventionally, to achieve scalable diagnosis of many analytes, single-analyte capture is enforced by a limiting dilution (Σₙλₙ [symbol] 0.1) such that individual partition measurements can be directly classified. In diagnostics, sample concentrations are unknown and can vary over multiple orders of magnitude. The inventors show how their framework tolerates multi-analyte capture such that samples of higher concentrations do not flood the inventors' system. The ability to diagnose with higher λ also reduces the need to generate many partitions. Enabling diagnostics with, for instance, hundreds of microchambers could enable scalable multiplexing on static, point-of-care formats. Asynchronous fingerprinting offers a second practical advantage. Many biosensing techniques are limited in M, the number of orthogonal measurements acquired from each partition, either due to spectral overlap or sensor cross-reactivity. However, more sensors may be necessary to assign unique fingerprints to each analyte. Asynchronous fingerprinting bypasses this limitation by enabling reconstruction of the signal from partial fingerprint measurements. Still, the inventors' previous theoretical and simulated results indicate that users should first maximize M and increase G as necessary (Kota et al., 2022).

The inventors' ddPCR assay has a few critical limitations that warrant future development. First and foremost, they designed probes to assign unique barcodes to the bacterial genera in the inventors' chosen panel. However, future LNA probe design must account for bacteria outside the panel that could plausibly appear in a sample to ensure that the designed barcodes are specific to the intended pathogens. These design requirements motivate new bioinformatics tools. In general, the inventors recommend that their framework is applied in applications where the scope of plausible analytes in the intended samples is well understood such that the specificity of the analytes' fingerprints can be verified. Second, they amplified the full 16S gene for maximum sequence flexibility in probe design. Amplifying the full length, 1500 base pair gene is well beyond the manufacturer recommendations. In ddPCR, longer extension times and high cycle counts can generally only help improve endpoint amplitudes and clustering. Here, the PCR protocol alone was over 8 hours which is not ideal for infection diagnostics. The primary goal was to demonstrate the inventors' mathematical framework such that the thorough optimization of this assay is beyond the scope of this report. Future iterations on the inventors' approach could either employ custom master mixes with faster polymerases or restrict the amplicon to a shorter 16S segment. Third, many clinical infections may be caused by bacteria or fungi. Multiplexing primers to include eukaryotic marker genes along with the 16S primers for bacteria could enable broadening the panel. All of the above lines of research should also consider the optimal marker genes to target with nonspecific probes; certainly, no single gene target will be optimal for all applications in microbial diagnostics.

While there is room for improvement in the ddPCR approach, expanding the measurement space with non-binary measurements would dramatically improve diagnostic performance as fewer sensors could assign unique fingerprints to microbes at a higher rate. Some of these non-binary effects may be readily present in the droplet “lean” and “lift” likely caused by weak interactions between probes and slightly mismatched amplicons. These potential sources of additional information were ignored by the hard thresholding employed in this work. Moreover, while the inventors present their framework in the context of microbial diagnostics where the inventors' research group is most familiar, they note the generality of the approach for other diagnostic applications. Combining conventional sensors with new techniques in microfluidics and signal processing will offer a suite of new interdisciplinary approaches to scalable, multiplexed biosensing.

### Example 3—Materials and Methods

Theory: Identifiability with Common Types of Sensors. For estimating λ, the property of identifiability means that there is a one-to-one correspondence between each realizable distribution of measurements and the Poisson rates λ: if p(y|λ)=p(y|λ′) for all y, then λ=λ′. From an optimization perspective, identifiability implies that the λ* is the unique global optimum to the likelihood function if the inventors have infinite measurements. Therefore, identifiability essentially confirms that the inventors' approach is possible and worth pursuing further.

Notation. The inventors use bold face upper and lower case letters for matrices and vectors, respectively. Non-bold, lower case letters represent scalars. They denote the vector of all zeros as 0 with its dimensionality dependent on context. They use script letters (A, B, etc.) to denote sets. They denote eⱼ as the standard basis vector with eⱼ=1 and eᵢ=0 for all i≠j. Let a and b be two arbitrary vectors of the same dimension, and let aᵢ and bᵢ denote their ith elements. They use supp(a) to denote the support of vector a defined as the index set where aᵢ>0 for i∈supp(a). The inventors use the notation a [symbol] b to imply that aᵢ≥bᵢ ∀i, and they use a [symbol] b to further imply the existence of at least one index i where aᵢ>bᵢ. A set in the subscript of a vector such as x_{A} refers to the subvector of x indexed by the elements of A. The inventors make frequent use of the shorthand Σa to denote the summation over elements of a vector a.

Definitions and Assumptions. The inventors treat the dataset of measurements from all partitions in a sensor group as samples of a random variable y. The signal (i.e., analyte quantities in a partition) x is N-dimensional with x˜Poisson(λ*). Each signal is measured by M sensors to yield the observation vector y (e.g., M fluorescence measurements). They define the function ƒ: [symbol] ₊^{N}→ [symbol] ^{M} that is composed of M scalar functions ƒₙ: [symbol] ₊^{N}→ [symbol] . A particular measurement value yₘ is determined by the sensor output ƒₘ(x) plus some additive, zero-mean random noise nₘ.

$$y = [ \begin{matrix}{y}_{1} \\ {y}_{2} \\ ⋮ \\ {y}_{M}\end{matrix} ] = [ \begin{matrix}{f}_{1} ( x ) \\ {f}_{2} ( x ) \\ ⋮ \\ {f}_{M} ( x )\end{matrix} ] + [ \begin{matrix}{n}_{1} \\ {n}_{2} \\ ⋮ \\ {n}_{M}\end{matrix} ] \qquad ( 4 )$$

Note that the sensor functions fm are group-dependent. For example, each group may have different probes.

The inventors assume that the inventors' sensors are monotonic and saturating and that the inventors' system obeys fingerprint equivalence. Given these properties, they prove sufficient conditions for identifiability. Without loss of generality, they will say that all M sensor functions obey these properties. An additional sensor that does not follow these properties does not nullify the inventors' analysis.

Definition 4.1 (Monotonic Sensors). A sensor function ƒₘ: [symbol] ₊^{N}→ [symbol] is monotonic increasing if a b [symbol] ƒₘ(a)≥ƒₘ(b) and monotonic decreasing if a [symbol] b⇒ƒₘ(a)≤ƒₘ(b).

Monotonic functions are very common and natural; for instance, many sensing modalities have a monotonic increasing sigmoidal response to their input. Notably, sensors may saturate in their response to a subset of analytes at which point increasing the analyte content does not change the output. The inventors define this formally:

[symbol]

Definition 4.2 (Saturating Sensors). If x [symbol] x′ and ƒ(x)=ƒ(x′) for a set of monotonic sensors, then for all z that satisfy z [symbol] z′ [symbol] x′ and supp(z−z′)⊆supp(x−x′), ƒ(z)=ƒ(z′).

Any analytes added to x′ to yield x, indexed by supp(x-x′), did not influence senor output measurements. These sensors are either saturated in their response to these analytes, or these analytes do not interact with the sensors at all. Because any addition of these components to x′ yields the same M measurements, any addition of them to a signal greater than or equal to x′ must also result in no change in sensor measurements for these sensors to be considered saturating.

The inventors define a final intuitive condition on the inventors' system called fingerprint equivalence. The fingerprint of analyte n is the measurement yielded by an isolated copy of the analyte, or ƒ(eₙ). Among analytes with identical fingerprint responses within a sensor group, the total number of occurrences of these analytes dictates the output response. In other words, the sensors treat these analytes as interchangeable copies of each other.

Definition 4.3 (Fingerprint Equivalence). Let [symbol] ⊆{1, . . . , N} be an index set of analytes with identical fingerprints, i.e. ƒ^{(e}^{ⱼ}⁾ is fixed for all i∈ [symbol] . A system has the fingerprint equivalence property if for any pair of vectors x and x′ with supp(x), supp(x′)⊆ [symbol] and Σₙxₙ=x′ₙ, the inventors have ƒ(x)=ƒ(x′).

Note that even if all analyte fingerprints are distinct, multiple signals can still map to the same measurement vector since the inventors allow for cases of multi-analyte capture in the same partition. The inventors define these signals as members of a collision set.

Definition 4.4 (Collision Sets). The collision set [symbol] ₓ for signal x is the set of all signals x that satisfy ƒ(x′)=ƒ(x).

The inventors define [symbol] as the set of unique collision sets. In 2-channel ddPCR with binarized measurements, there are four collision sets in each sensor group ({0,1}²). It will soon be clear that observations y are drawn from a mixture model. The inventors can define each mixture element as follows:

Definition 4.5 (Mixture Element). The mixture element Ex for signal x is the set of all signals x′ that satisfy p(y|x)˜p(y|x′).

Note with any zero-mean noise, p(y|x)˜p(y|x′)⇒ƒ(x)=ƒ(x′) such that εₓ⊆ [symbol] ₓ. In some cases, such as additive white Gaussian noise, εₓ⊆ [symbol] ₓ. The inventors define ν as the set of unique mixture elements with arbitrary εₓ∈ν.

Proof of Identifiability. With G different sensor groups indexed by g, the inventors assume that the sensor group applied to a measurement y is known and deterministic. Each sensor group has a different function ƒ that maps x to M-dimensional space (e.g., different probes in ddPCR). Identifiability means that p(y|λ)=p(y|λ′) ∀y,g⇒λ=λ′. Each λ must yield a unique set of G distributions of measurements.

The inventors will use the notation [symbol] ᵤ^{g} and εᵥ^{g} to specify the group g when necessary. For an arbitrary group, they can express p(y|λ) as:

$$p ( y \vert λ ) = {\sum }_{x} p ( y \vert x ) P ( x \vert λ ) \qquad ( 5 )$$

$$= {\sum }_{{ℰ}_{𝓋} ∈ 𝒱} p ( y \vert x ∈ {ℰ}_{𝓋} ) P ( {ℰ}_{𝓋} \vert λ ) \qquad ( 6 )$$

$$P ( {ℰ}_{𝓋} \vert λ ) = {\sum }_{x ∈ {ℰ}_{𝓋}} P ( x \vert λ ) . \qquad ( 7 )$$

If a mixture distribution is identifiable, it means that identical distributions must come from the same set of weights on the mixture elements; in this context, p(y|λ)˜p(y|λ′)⇒P(εᵥ|λ)=P(εᵥ|λ′) ∀v. Many finite mixtures (what the inventors practically have in MMVP) and countably infinite mixtures with common noise distributions are identifiable (Yakowitz & Spragins, 1968; Yang & Wu, 2013), and they assume that the system noise characteristics lend to an identifiable mixture. However, the inventors need to prove the identifiability of MMVP, or that equal mixture weights implies equal Poisson parameters: P(εᵥ^{g}|λ)=P(εᵥ^{g}|λ′) ∀v,g⇒λ=λ′. Note that because εᵥ^{g}⊆ [symbol] ᵤ^{g} and unique collision sets are disjoint, P(εᵥ^{g}|λ)=P(εᵥ^{g}|λ′) ∀v,g⇒P( [symbol] ᵤ^{g}|λ)=P( [symbol] ᵤ^{g}|λ′) ∀u,g.

The inventors assume P( [symbol] ᵤ^{g}|λ)=P( [symbol] ᵤ^{g}|λ′) ∀u,g and prove the implication of λ=λ′ given a set of monotonic, saturating sensors and with a system exhibiting fingerprint equivalence in all G groups. They first focus on what can be concluded from a single, arbitrary sensor group (dropping the g superscript) with analytes potentially having nonunique fingerprints, and then they conclude with how multiple groups can be pooled to achieve identifiability. Without loss of generality, the inventors' analysis will focus on monotonic increasing sensors.

Define [symbol] ⊆{1, . . . , N} that is indexed by a where there exists some m such that ƒₘ(eₐ)>ƒ(0). Define the complementary set [symbol] indexed by b where ƒ(e_{b})=ƒ(0).

Lemma 4.1. ƒ(x)=ƒ(0) if and only if xₐ=0 ∀a∈

[symbol]

Proof: Let one define the shorthand x_{B}∈{x:xₐ=0 ∀a∈ [symbol] }. Note supp(x_{B})⊆ [symbol] . By definition, ƒ(e_{b})=ƒ(0) for any b∈ [symbol] . Let c be an arbitrary positive integer. Monotonic functions allow for ƒ(ce_{B}) [symbol] ƒ(e_{b}) which could lead to a nonzero sensor response. However, because the [symbol] inventors' functions are also saturating, ƒ(ce_{b})=ƒ(0) since supp(ce_{b}−e_{b}) S supp(e_{b}−0). Next, because of fingerprint equivalence, any signal x_{B} with supp(x_{B})⊆ [symbol] and with a sum Σx_{B}=c satisfies ƒ(x_{B})=ƒ(ce_{b}). Therefore, ƒ(x_{B})=ƒ(0).

The inventors prove the forward condition, ƒ(x)=ƒ(0)⇒xₐ=0 ∀a∈ [symbol] , by contradiction. Say ƒ(z)=ƒ(0) and let z satisfy zₐ≥1 for some a∈ [symbol] . By definition of [symbol] , ƒ(eₐ)>ƒ(0), and z [symbol] eₐ. [symbol] With monotonic functions, ƒ(z) [symbol] ƒ(eₐ) [symbol] ƒ(0) and they have arrived at a contradiction.

This result indicates that the values of x_{B} do not influence the measurement output of any signal because sensors are saturating. In other words, for an arbitrary signal z and some x_{B}, ƒ(z+x_{B})=ƒ(z) because supp(z+x_{B}−z)=supp(x_{B}−0) and ƒ(x_{B})=ƒ(0). The values of elements in x_{B} are therefore entirely arbitrary for the analysis of collision sets.

Lemma 4.2. If P( [symbol] ₀|λ)=P( [symbol] ₀|λ′), then [symbol] =

[symbol]

Proof: By Lemma 4.1, [symbol] ₀ contains all x with [symbol] =0 with arbitrary values on [symbol] . Therefore, P( [symbol] ₀|λ)=P( [symbol] ₀|λ′) implies

P( [symbol] =P(x [symbol] =0|λ′) (8)

[symbol]

= [symbol] (9)

Σ [symbol] =Σ [symbol] (10)

Lemma 4.3. If for x∈ [symbol] ₑ_{ₐ}, xₐ is the only nonzero value in x [symbol] , then λₐ=λₐ′.

Proof. For identifiability, the inventors assume P( [symbol] ₑ_{ₐ}|λ)=P( [symbol] ₑ_{ₐ}|λ′). By definition of [symbol] , ƒ(eₐ)>ƒ(0). If xₐ is the only nonzero value of x [symbol] , one of two possibilities exists. If ƒ(2eₐ)>ƒ(eₐ), then only eₐ is in its own collision set because of monotonicity. Similarly, if ƒ(2eₐ)=ƒ(eₐ), then ƒ(ceₐ)=ƒ(eₐ) for any positive integer c because sensors are saturating. If the former condition holds, then P( [symbol] ₑ_{ₐ}|λ)=P( [symbol] ₑ_{ₐ}|λ′) implies

P(eₐ| [symbol] =P(eₐ| [symbol] ) (11)

λₐ [symbol] _{=λ}ₐ′ [symbol] (12)

Lemma 4.2 implies that λₐ=λ′ₐ. Similarly, if the latter condition holds, then P( [symbol] ₑ_{ₐ}|λ)=P( [symbol] ₑ_{ₐ}|λ′) implies

P( [symbol] ₑ_{ₐ}| [symbol] =P( [symbol] ₑ_{ₐ}| [symbol] ) (13)

(1−e^{-λ}^{ₐ}) [symbol] =(1−e^{-λ}^{ₐ}^{′}) [symbol] (14)

Σ [symbol] =Σ [symbol] (15)

Lemma 4.2 coupled with this last equality means that λₐ=λₐ′, thus concluding the proof.

From here, the inventors first derive results for the special case where all analytes indexed by [symbol] have unique singlecopy fingerprints. Afterwards, they generalize to multiple groups, allowing for equal nonzero fingerprints within a group. The next Lemma guarantees at least one index a to which Lemma 4.3 can be applied.

Lemma 4.4. If ƒ(eᵢ)≠ƒ(eⱼ) ∀i,j∈ [symbol] with i≠j, ∃a∈ [symbol] such that x∈ [symbol] ₑ_{ₐ} are nonzero in x [symbol] only on index a.

Proof: First, with unique nonzero fingerprints in A and monotonic sensors, the fingerprint responses ƒₘ(eₐ) can be sorted. Without loss of generality, the inventors allow all functions to be monotonic increasing. Starting arbitrarily with m=1, they can select the minimal set [symbol] ⊆ [symbol] that minimizes ƒ₁(eₐ) such that ∀a∈ [symbol] , ∀j∈ [symbol] ^{c}, ƒ₁(eₐ)<ƒ₁(eⱼ). If | [symbol] |>1, then the process can be repeated with m=2 (and so forth) on the subset [symbol] until there is one unique minimum and its corresponding index a.

For this eₐ, all i∈ [symbol] \a satisfy ƒₘ(eᵢ)>ƒₘ(eₐ) for at least one m. Therefore, signals in the collision set x∈ [symbol] ₑ_{ₐ} must satisfy xᵢ=0 for all i. Signals with at least one xᵢ≥1 would have at least one m where ƒₘ(x)>ƒₘ(eₐ), and therefore not be in the collision set by definition. This conclusion completes the proof by contradiction.

Next, the inventors show how this result chains to all analytes indexed in [symbol] .

Lemma 4.5. If ƒ(eᵢ)≠ƒ(eⱼ) ∀i,j∈ [symbol] with i≠j, λₐ=λₐ′ ∀a∈ [symbol] .

Lemmas 4.3 and 4.4 guarantee at least one a that yields λₐ=λₐ′. Let one call this index a₁ and define the subset [symbol] ⊆ [symbol] , the subset of indices for which λᵢ=λᵢ′ ∀i∈ [symbol] . At this point, [symbol] ={a₁}. Repeating the process in the proof of Lemma 4.4, the inventors can find a new index a₂ that satisfies ƒₘ(eᵢ)>ƒₘ(eₐ_{₂}) ∀i∈ [symbol] ^{c}\a₂ for at least one m. They will use the shorthand e₂≡eₐ_{₂}.

For the direct proof of identifiability, the inventors assume P( [symbol] ₑ₂|λ)=P( [symbol] ₑ₂|λ′), or:

$${\sum }_{x ∈ {C}_{{e}_{2}}} P ( x \vert λ ) = {\sum }_{x ∈ {C}_{{e}_{2}}} P ( x \vert {λ}^{′} ) . \qquad ( 16 )$$

Among signals x in [symbol] ₑ_{₂}, xₙ=0 for n∈ [symbol] ^{c}\a₂ because ƒₘ(eₙ)>ƒ(e₂) for some m, and sensors are monotonic. These signals can also be partitioned into those with xₐ_{₂}=0, and those with xₐ_{₂}≥1. If any of the former type exist, xᵢ>0 for some of the indices i∈ [symbol] . These signals' terms in the summation follow the form [ [symbol] P(xₙ|λₙ)] [symbol] . Note that λᵢ=λᵢ′ ∀i∈ [symbol] combined with Lemma 4.2 yields [symbol] λᵢ= [symbol] λᵢ′. Because λₙ=λₙ′ for n∈ [symbol] , the product component is equal on both sides as well. Therefore, these terms can be eliminated in Equation (16). The inventors will denote the set of remaining x in the summation as [symbol] ′.

The second type of signals in [symbol] ₑ_{₂} can be considered in two cases: xₐ_{₂}=1 only, and xₐ_{₂}≥1. This case partitioning follows the same logic in the proof of Lemma 4.3. In both cases, some signals may satisfy xᵢ≥0 with i∈ [symbol] . For these signals, ƒ(x)=ƒ(e₂) regardless of the values of xᵢ such that P(xᵢ|λᵢ) can be ignored. The inventors only need to consider the i∈ [symbol] that satisfy xᵢ=0. For the case where xₐ_{₂}=1 only, Equation 16 simplifies to

$${\sum }_{x ∈ {C}^{′}} ( {e}^{- \sum } \text{?} ) ( {e}^{- \sum } \text{?} ) λ \text{?} = {\sum }_{x ∈ {C}^{′}} ( {e}^{- \sum } \text{?} ) ( {e}^{- \sum } \text{?} ) λ \text{?} \qquad ( 17 )$$

$$\text{?} \text{indicates text missing or illegible when filed}$$

The exponential terms are equal on both sides of the equation by previous conclusions and can be distributed out of the sum and divided, yielding λₐ_{₂}=λ′ₐ_{₂},

If xₐ_{₂}≥1, Equation 16 simplifies instead to:

$${\sum }_{x ∈ {C}^{′}} ( {e}^{- \sum } \text{?} ) ( {e}^{- \sum } \text{?} ) ( 1 - e \text{?} ) = {\sum }_{x ∈ {C}^{′}} ( {e}^{- \sum } \text{?} ) ( {e}^{- \sum } \text{?} ) ( 1 - e \text{?} ) \qquad ( 18 )$$

$${\sum }_{x ∈ {C}^{′}} ( {e}^{- \sum } \text{?} - {e}^{- \sum } \text{?} ) = {\sum }_{x ∈ {C}^{′}} ( {e}^{- \sum } \text{?} - {e}^{- \sum } \text{?} ) \qquad ( 19 )$$

$$\text{?} \text{indicates text missing or illegible when filed}$$

Because [symbol] λᵢ= [symbol] λᵢ′, this also yields λₐ_{₂}=λₐ_{₂}′. Now, a₂ can be added to [symbol] and the process can be repeated until [symbol] = [symbol] such that [symbol] = [symbol] .

Now, the inventors will extend this result to the case of having equal fingerprints in the same sensor group, i.e. that ƒ(eᵢ)=ƒ(eⱼ) for some pairs i,j.

Lemma 4.6. Define the disjoint sets [symbol] ₁, [symbol] ₂, . . . [symbol] _{C} indexed by c with ∪_{c=1}^{C} [symbol] _{c}= [symbol] such that for all i,j∈ [symbol] _{c}, ƒ(eᵢ)=ƒ(eⱼ). Then, [symbol] = [symbol] .

Proof: Note that the Poisson distribution has the property that if xᵢ are each independently drawn from Poisson(λᵢ), then [symbol] xᵢ˜Poisson ( [symbol] ). The inventors can then simply define a dummy variables x_{c}^{†}= [symbol] xᵢ and λ_{c}^{†} such that x_{c}^{†}˜Poisson(λ_{c}^{†}). This dummy variable represents an “analyte” that appears with a distribution governed by the total quantities of analytes with the same fingerprint. However, what matters for identifiability is the sensor functional values of signals, i.e. that ƒ(a)=ƒ(b) if for all c, [symbol] = [symbol] . Namely, x_{c}^{†}˜Poisson(λ_{c}^{\}) by fundamental [symbol] properties of the Poisson distribution, but it is only with the condition of fingerprint equivalence (Definition 4.3) that let one apply all previous results that are based on collision sets, i.e., sets of signals with equal functional values. These yield λ_{c}^{†}=λ′_{c}^{†}′, or [symbol] = [symbol] ∀c.

Theorem 4.7. Let g index G different sensor groups that satisfy fingerprint equivalence and that contain monotonic, saturating sensors. For each group g, define the row vector z_{c}^{g} of zeros and ones with ones in the indices associated with [symbol] _{c}. Define the N-column matrix Z_{g} whose C rows are comprised of z_{c}^{g} ∀c. Define the N-column matrix Z as the vertical concatenation Z_{g} ∀g. If rank(Z)=N, then λ=λ′.

Proof: This theorem is a formal way of saying that Lemma 4.6 must yield N independent equations when applied to all groups where the sensing and system conditions hold. The inventors can consider the system of equations yielded by Lemma 4.6 and represented by Zλ=Zλ′, or Z(λ−λ′)=0. If rank(Z)=N, then it follows that λ=λ′. Therefore, they have P( [symbol] ᵤ^{g}|λ)=P( [symbol] ᵤ^{g}|λ′) ∀(u,g)⇒λ=λ′, concluding the inventors' proof of identifiability. use Bacterial Panel. The inventors ordered bacterial species' genomic DNA from the American Type Culture Collection (ATCC, Manassas, VA). The species' names and their ATCC identifiers are as follows: A. baumannii (BAA-1605), B. fragilis (25285), E. cloacae (13047), E. faecium (BAA-23200, E. coli (11775), K. pneumoniae (13883), P. aeruginosa (BAA-1744), S. aureus (12600), S. epidermidis (14990), S. saprophyticus (15305), S. agalactiae (13813), and S. pneumoniae (33400). Particular strains were selected based on their availability at the time of purchase and only if ATCC provided whole genome sequence information for the isolate.

Probe Design. All oligonucleotides were acquired from Integrated DNA Technologies (Coralville, IA) with HPLC purification. The inventors used ThermoBLAST from DNA Software (Plymouth, MI) to align the 16S primers (27F and 1492R from (Johnson et al., 2019), Table 1) against bacterial genomes and find amplicons. The inventors passed these amplicons to a custom Matlab script to design probes. They needed probes with a melting temperature (Tₘ) a few degrees higher than that of the primers, targeting at least 65° C. Probes for barcoding must hit multiple bacterial taxa, and shorter probes are naturally less specific. LNA's increase probe melting temperature in a highly position-specific manner. To avoid combinatorially increasing the inventors' probe search space, the inventors deferred LNA positioning until after sequence selection. They used heuristics to filter for probes that would have sufficient Tₘ with some flexibility in LNA positioning. They chose a sequence length of 11 nucleotides with 5-8 GC nucleotides, without four consecutive G's or C's, and without a G on the 5′ end to avoid self-quenching of the fluorophore. They used Smith-Waterman alignment in Matlab to pre-screen for probes that self-hybridize and to assess cross-hybridization of probes amongst the evolving candidate set. To assist in achieving near binary measurements, the inventors considered perfect matches on all 16S copies to be “1” for a genome, and for imperfect homology, they filtered for sequences neither had nine consecutive matches nor a single G-T mismatch. This latter filtering is a proxy for ensuring that probes have weak, negligible interactions against 16S sequences where they do not have perfect complementarity. The former filtering for positive hits was intended to avoid the issue of mixtures of barcodes for any particular bacteria for simplicity in the inventors' initial demonstration.

TABLE 2
Oligonucleotides used in this study.*

| | | SEQ |
|---|---|---|
| Component | | ID |
| ID | Sequence | NO: |
| Forward | AGAGTTTGATCMTGGCTCAG | 1 |
| Primer | | |
| 27F | | |
| Reverse | TACGGYTACCTTGTTAYGACTT | 2 |
| Primer | | |
| 1492R | | |
| Probe 1 | TA + A + C + GGC + T + C + AC | 3 |
| Probe 2 | CTT + T + CGC + C + C + AT | 4 |
| Probe 3 | A + TT + C + C + GA + CT + TC | 5 |
| Probe 4 | A + C + C + AA + T + C + CATC | 6 |
| Probe 5 | A + A + G +CA + C + TCCGC | 7 |

* - A “+” preceding a base indicates that the LNA analogue of the base was used. Probes 1, 2, and 5 were tagged with HEX and Probes 3 and 4 with FAM on the 5′ ends. The dark quencher Iowa Black FQ was placed at the 3′ ends.

Given a set of filtered, candidate probes, the inventors used a coordinate ascent strategy to iteratively optimize a set. They hypothesized that barcoding the full-length 16S gene with probes could achieve genus level resolution, as achieving below this level with sequencing of a subsection of the gene is often limited to genus information. As a result, the inventors encouraged similarity use Z 129 of the three Staphylococcus species and the two Streptococcous species. Define [symbol] as a set of pairs of bacteria (bᵢ,bⱼ) within a genus that are similar. The complementary set [symbol] includes all other [symbol] bacterial pairs. Let k_{p,i} represent the 11-mer barcode of bacteria i with probes indexed by p. Coordinate ascent sought to solve

$$arg {max}_{p} [ {\sum }_{( {b}_{i} , {b}_{j} ) ∈ 𝒮} - {‖ {k}_{p , i} - {k}_{p , j} ‖}_{2}^{2} + log ( {\sum }_{( {b}_{i} , {b}_{j} ) ∈ 𝒟} {‖ {k}_{p , i} - {k}_{p , j} ‖}_{2} ) ] + θ {min}_{( {b}_{i} , {b}_{j} ) ∈ 𝒟} {‖ {k}_{p , i} - {k}_{p , j} ‖}_{2} \qquad ( 20 )$$

The first term is taken from research in metric learning (Xing et al., 2002), and the second term (with a weight of θ=10) highly rewards some nonzero separation between all bacterial pairs that are intended to be discriminated between. The inventors chose an initial random set of probes that passed the inventors' cross-hybridization check. They iteratively cycled through a shuffled order of the candidate set of probes, evaluating one probe at a time for replacement with any of the other probes that passed the initial filtering step. If replacing a probe improved the objective function, the probe set was updated and the search continued. The algorithm terminated when all probe sequences had been evaluated for replacement but not replaced. For the chosen set of sequences, the inventors evaluated the alignment against 16S genes with imperfect homology (the zeroes in the barcodes). As much as possible, they positioned LNAs at mismatch sites to improve the thermodynamic discrimination against these sequences. They evaluated all Tₘ's in IDT's OligoAnalyzer, positioning additional LNAs as necessary to reach a sufficient probe Tₘ. Final probe sequences are listed in Table 3.

Preparation of Bacterial Samples. First, the inventors prepared monomicrobial dilutions of genomic DNA in MilliQ purified water. One dilution was prepared for each bacteria, approximately targeting a concentration λ between 0.2 and 2 (“Concentration 1”). They diluted each of these by ½ to yield a second dilution of “Concentration 2.” They used a custom script to assign random combinations of these bacterial dilutions to samples, generating five samples with k=2 unique bacteria and six samples of k=3 and k=4. They reserved one sample to be a no template control (NTC, water alone). The probability of drawing each bacteria was adaptively weighted to encourage approximately even representation of each taxa across the samples (note, Staphylococcus and Streptococcus species were lumped and treated as one taxa each to not overrepresent their constituent species).

Droplet Digital PCR. Primers were at 900 nM and all probes were at 125 nM. The inventors used Bio-Rad's ddPCR Multiplex Supermix and prepared master mixes, generated droplets, and read out droplets according to the manufacturer's instructions. For PCR cycling, extension times were set to 7 minutes because of the long amplicon (approximately 1500 base pairs) that is atypical in ddPCR, partly following guidance from (Lasham et al., 2020) and internal data.

PCR cycling was as follows: 95° C. for 10 minutes (initial denaturation and hot-start deactivation), 60 cycles of 94° C. for 30 seconds (denaturation) and 60° C. for 7 minutes (anealing and extension), 98° C. for 10 minutes. Ramp rates during cycling were set to 2° C./second. Samples were refrigerated at 4° C. for 30 minutes prior to droplet readout.

Plate Arrangement and Manual Thresholding. FIG. 20 illustrates how the inventors arranged the samples in the inventors' ddPCR plate. The first two rows serve as reference samples, allowing one to estimate the ground truth concentration of the two dilutions based on the proportion of empty droplets. Empty droplets were determined by manual thresholding. These are also color coded to reflect which group mixture was applied to each. The amplitudes of positive droplets in the reference rows, and their anticipated partial barcodes, were used to guide manual thresholding cutoffs for the rest of the plate. Each polymicrobial sample was distributed across four wells each assigned to a probe group (“G1” for Group 1, etc.).

For manual thresholding, the inventors pooled the data from the reference wells for each group and overlaid a particular well's data (FIGS. 21A-B). Due to some mild “lean” and “lift” of the raw ddPCR clusters caused likely by partial probe interactions, they allowed any linear threshold for each fluorescence channel determined by two user-selected points. The user confirmed the threshold selection (FIG. 21C). This process was repeated for all wells, including the reference wells, and conducted twice for each well to define thresholds for both HEX and FAM. Ultimately, all droplets from each well are converted to two dimensional binary measurements.

Settings for SPoRe. The inventors set the initial value of λ to the constant vector 0.1 for an unbiased initialization. The inventors performed gradient descent to minimize the negative log likelihood with backtracking line search to adaptively tune the learning rate at each iteration. After computing the gradient and projecting to λₙ=max(λₙ, 10⁻⁶), SPoRe checks if the negative log likelihood decreased. Values of λₙ=0 cause numerical issues in gradient computations, hence the small offset. If it did not, the iteration's step size was cut in half. SPoRe only commits to an update to λ after confirming a reduction in the negative log likelihood, at which point it resets the learning rate and proceeds to the next iteration. SPoRe terminates when the log likelihood's relative change over the previous five iterations falls below 10⁻⁶. In contrast to the inventors' original implementation of SPoRe (Kota et al., 2022), the inventors use an exact gradient computation over the entire dataset made feasible by the nature of ddPCR data (Section 5.2) rather than a Monte Carlo approximation over a batch of data.

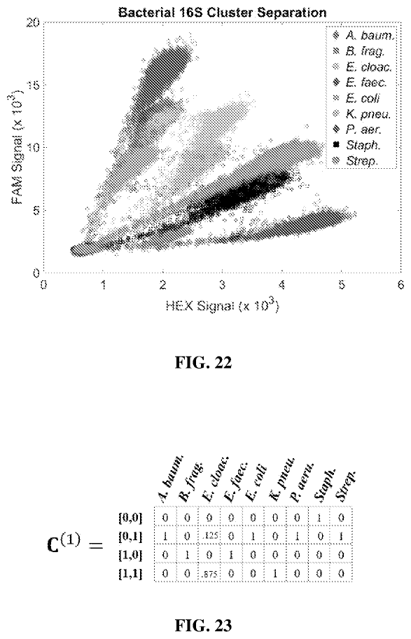

Assessing E. cloacae Barcode Variability. E. cloacae's amplicons appeared to always interact with Probes 3 and 4, but a small subcluster appeared to lack the HEX response to Probe 1 (FIG. 22A). The inventors used the inventors' SPoRe algorithm to estimate the barcode abundances in reference wells A3 and B10 (FIG. 20) which both contained Probe 1. After manual thresholding, the binarized data and “analyte” with barcodes [0,1], [1,0], and [1,1] were passed to SPoRe, and SPoRe estimated the abundances of the amplicons with these responses. In this case, the [1,0] quantity was nearly zero, consistent with the expectation that E. cloacae always interacted with a FAM probe. The fraction of amplicons with the HEX probe was determined by λ_{[1,1]}/(λ_{[1,1]}+λ_{[0,1]}). In A3 and B10, these were estimated to be 0.832 and 0.828, respectively. The inventors' sequence analysis found eight 16S copies in the E. cloacae genome, so it is possible that one amplicon had a sequencing error such that Probe 1 truly binds to 7/8 copies. Therefore, column 3 of matrix C had 0.875 of barcode 3 and 0.125 of barcode 1 (3a).

Amplitude Multiplexing with More than Two Probes. Amplitude multiplexing is a technique to resolve more probes than the available number of color channels, but it is typically used with each unique probe participating in an orthogonal assay with its own primer pair (Whale et al., 2016). Probe concentrations can be adjusted to “move” the cluster positions. Here, the inventors adjust probe concentrations with a single pair of primers. Probes 1 and 5 were tagged with HEX, and Probes 2-4 were tagged with FAM (FIG. 14A). The inventors tested a few concentration variations (results not shown), and the data depicted here is with Probes 1, 2, and 4 at 125 nM, and with Probes 3 and 5 at 250 nM. Based on each 16S gene's barcode, droplets containing that gene will position in clusters whose channel intensities roughly correlate with the total probe concentration tagged with the corresponding fluorophore.

Exact Gradient Computation and p(y|x) Model for ddPCR. The inventors first focus on the gradient resulting from a single probe group. In a single group, there are only four viable measurements with y∈{0,1}². Let one define [symbol] ={0,1}², and p_{y} as the proportion of the D measurements that equal y. The inventors can then re-express Equation (2) as:

$$\begin{matrix}{λ}_{MLE} & = & {arg max}_{λ} \frac{1}{D} {\sum }_{d = 1}^{D} log {\sum }_{x ∈ {ℤ}_{+}^{N}} p ( {y}_{d} \vert x ) P ( x \vert λ ) & ( 21 ) \\ & = & {arg max}_{λ} {\sum }_{y ∈ 𝒴} {p}_{y} log {\sum }_{x ∈ {ℤ}_{+}^{N}} p ( {y}_{d} \vert x ) P ( x \vert λ ) & ( 22 ) \\ & & & \end{matrix}$$

Here, the inventors will use λ≡λ^{(bact)} since they will be optimizing over the bacterial concentrations directly. In this case, E. cloacae is the only bacteria with a fractional abundance of a probe binding site—approximately 87.5% of its copies interact with probes 1, 3, and 4, and 12.5% interact with only probes 3 and 4 (Section 4.8). Similarly to how the inventors defined C in Section 2. 1, they can define C^{(g)} for group g with each bacterium's fractional abundances of genes with a corresponding barcode. FIG. 23 shows an example of C⁽¹⁾, which can be generated with FIG. 14A as a reference.

The inventors define p(y|x)=Πₘp(yₘ|x). For p(yₘ=1|x), then p(yₘ|x)=1 if the droplet has at least one copy of a gene that interacts with probe m and p(yₘ|x)=0 otherwise. For p(yₘ=0|x), this likelihood is 1 if none of the genes in the droplet interact with probe m and 0 otherwise.

However, with the analyte current defined as a copy of the nth bacterium's 16S gene, the inventors must be careful. For instance, with index 3 corresponding with E. cloacae, if x₃=1 in x, p(y₁|x) may not be 1 since one copy of E. cloacae's 16S gene is not guaranteed to interact with probe 1. To resolve this, they will temporarily transform the problem to the space of gene barcodes for this group: λ^{(BC}^{₁}⁾=C⁽¹⁾λ. Note λ^{(BC}^{₁}⁾ is 4dimensional. The inventors can define x^{(BC}^{₁₎}=[x₀₀,x₀₁,x₁₀,x₁₁]^{T} as the vector representing the quantities of 16S genes from any source bacteria that interact with the probes in the pattern noted in the subscript, noting x^{(BC}^{₁}⁾˜Poisson(λ^{(BC}^{₁}⁾). Lastly, let one define [symbol] _{y} as that set where if x^{(BC}^{₁}⁾∈ [symbol] _{y}, then p(y|x^{(BC}^{₁}⁾)=1. Now one can rewrite Equation (22) as:

$$= {\sum }_{y ∈ 𝓎} {p}_{y} log {\sum }_{{x}^{( {BC}_{1} )} ∈ {𝒳}_{y}} P ( {x}^{( {BC}_{1} )} \vert {λ}^{( {BC}_{1} )} ) . \qquad ( 23 )$$

The linearity of gradients allow one to treat this one y at a time, summing the contributions from each y at the end. In general, treat 00 as short for [0,0], 01 for [0,1], etc. Let ∇_{λ}⁰⁰ be the component of the gradient from y=[0,0], ∇_{λ}⁰¹ from y=[0,1], etc. The inventors will similarly define the mean log likelihood contributions as [symbol] ⁰⁰, [symbol] ⁰¹, etc. Similarly, define the rows of C⁽¹⁾ as C₀₀⁽¹⁾, C₀₀⁽¹⁾, etc. By convention, λ and other vectors should be assumed to be column vectors, but the rows of C⁽¹⁾ are row vectors. Thus, the inventors have:

[symbol]

⁰⁰=p₀₀ log P(x₀₁=0 and x₁₀=0 and x₁₁=0) (24)

=p₀₀ [symbol] ^{-x}^{₀₁}^{-x}^{₁₀}^{-x}^{₁₁} (25)

=p₀₀(−C₀₁⁽¹⁾−C₁₀⁽¹⁾−C₁₁⁽¹⁾)λ (26)

∇_{λ}⁰⁰=p₀₀(−C₀₁⁽¹⁾−C₁₀⁽¹⁾−C₁₁⁽¹⁾)^{T}. (27)

In the first line, the inventors define the conditions for x^{(BC}^{₁}⁾∈ [symbol] ₀₀ and solve. Genes that interact with either probe cannot be in droplets that yield y=[0,0]. Next, for the y=[0,1] response, at least one gene that interacts with the 2nd (FAM) probe must be present, and genes that interact with the HEX probe must be absent.

$$\begin{matrix}{ℓ}^{01} & = & {p}_{01} log P ( {x}_{01} \geq 1 and {x}_{10} = 0 and {x}_{11} = 0 ) & ( 28 ) \\ & = & {p}_{01} log ( 1 - {e}^{- {x}_{01}} ) {e}^{- {x}_{10} - {x}_{11}} & ( 29 ) \\ & = & {p}_{01} ( log ( 1 - {e}^{- {C}_{01}^{( 1 )} λ} ) - {C}_{10}^{( 1 )} λ - {C}_{11}^{( 1 )} λ ) & ( 30 ) \\ {∇}_{λ}^{01} & = & {p}_{01} [ ( \frac{{e}^{- {C}_{01}^{( 1 )} λ}}{1 - {e}^{- {C}_{01}^{( 1 )} λ}} ) {C}_{01}^{( 1 ) T} - {C}_{10}^{( 1 ) T} - {C}_{11}^{( 1 ) T} ] . & ( 31 )\end{matrix}$$

A virtually identical simplification for ∇_{λ}¹⁰ is omitted here. Lastly, for y=[1,1], the inventors have:

[symbol]

¹¹=p₁₁ log(P(x₁₁≥1)+P(x₁₁=0 and x₀₁>0 and x₁₀>0) (32)

=p₁₁ log((1−e^{-x}^{₁₁})+e^{-x}^{₁₁}(1−e^{-z}^{₀₁})(1−e^{-x}^{₁₀})). (33)

The remaining algebraic steps are omitted, but the final result is

$${∇}_{λ}^{11} {( = p )}_{11} {e}^{- {x}_{11}} \frac{\begin{matrix}- {C}_{11}^{( 1 ) T} - {e}^{- {x}_{01}} ( - {C}_{01}^{( 1 ) T} - {C}_{11}^{( 1 ) T} ) - \\ \begin{matrix}{e}^{- {x}_{10}} ( - {C}_{10}^{( 1 ) T} - {C}_{11}^{( 1 ) T} ) - \\ {e}^{- {x}_{01} - {x}_{10}} ( - {C}_{01}^{( 1 ) T} - {C}_{10}^{( 1 ) T} - {C}_{11}^{( 1 ) T} )\end{matrix}\end{matrix}}{( 1 - {e}^{- {x}_{11}} ) + {e}^{- {x}_{11}} ( 1 - {e}^{- {x}_{01}} ) ( 1 - {e}^{- {x}_{10}} )} \qquad ( 34 )$$

where Cᵢⱼ⁽¹⁾λ can be substituted for any xᵢⱼ.

The inventors can now say that for group 1, ∇_{λ}⁽¹⁾=∇_{λ}⁰⁰+∇_{λ}⁰¹+∇_{λ}¹⁰+∇_{λ}¹¹. The above process can be repeated for any group g. Therefore, the final gradient vector (arbitrarily scaled) is

$${∇}_{λ} = {\sum }_{g} {p}_{g} {∇}_{λ}^{( g )} \qquad ( 35 )$$

where p_{g} is the proportion of total droplets that come from group g.

Special Cases of MLE with ddPCR. From Equation (3) describing the generalized gradient in MLE, the inventors consider two commonly employed special cases. First, if samples are sufficiently dilute such that partitions are either empty (x_{d}=0) or have only one analyte, the goal is often to identify each nonzero signal independently with a classification process. In other words, assays must be designed such that p(y_{d}|x)>0 only for x*_{d}—the measurements are unambiguous.

Setting the gradient equal to zero and simplifying leads to

$${\hat{λ}}_{M L E} = \frac{1}{D} {Σ}_{d = 1}^{D} {x}_{d}^{*} .$$

In practice, clusters of classes must have reliable decision boundaries and concentrations are estimated by totaling the classification results.

The second specialized case is common for ddPCR where for each PCR assay is specific for a target analyte and assigned to a particular channel. With M channels, N=M and each measurement unambiguously determines the presence or absence of each target sequence. Precisely, p(y_{d}|x) is one or zero, and considerations of each analytes' quantity xₙ can be simplified to xₙ=0 (absent) or xₙ>0 (present). Each analyte n can be inferred independently such that

$${λ}_{n} = - log \frac{{D}_{0 , n}}{D}$$

where D_{0,n} is the number of droplets that do not contain analyte n. Each analyte's concentration is based on the proportion of droplets that do not analyte. This formula can be found by applying the above assumptions, setting Equation (3) to zero, and simplifying.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Aghazadeh et al., “Universal microbial diagnostics using random DNA probes,” Sci. Adv, vol. 2, no. 9, p. e1600025, September 2016.
Alam & Zhang, “A survey: Non-orthogonal multiple access with compressed sensing multiuser detection for mMTC,” doi.org/10.1155/2021/8840519, 2018a.
Alam & Zhang, “Enhanced compressed sensing based multiuser detection for machine type communication,” IEEE Wireless Communications and Networking Conference, WCNC, pp. 1-6, 2018b.
Alseekh et al., “Mass spectrometry-based metabolomics: a guide for annotation, quantification and best reporting practices,” Nature Methods, vol. 18, no. 7, pp. 747-756, 2021.
Bacconi et al, “Improved Sensitivity for Molecular Detection of Bacterial and Candida Infections in Blood,” J. Clin. Microbiol, vol. 52, no. 9, pp. 3164-3174, 2014.
Balkan et al., “Localization of more sources than sensors via jointly-sparse bayesian learning,” IEEE Signal Process. Lett., vol. 21, no. 2, pp. 131-134, 2014.
Baraniuk, “Compressive sensing,” Signal Processing Magazine, IEEE, no. July, pp. 118-121, 2007.
Baron et al., “Distributed compressive sensing,” doi.org/10.48550/arXiv.0901.3403, January 2009.
Bashir, “BioMEMS: State-of-the-art in detection, opportunities and prospects,” Advanced Drug Delivery Reviews, vol. 56, no. 11, pp. 1565-1586, 2004.
Basu, “Digital assays part I: Partitioning statistics and digital PCR,” SLAS Technology, vol. 22, no. 4, pp. 369-386, August 2017.
Brink et al., “DdPCRclust: An R package and Shiny app for automated analysis of multiplexed ddPCR data,” Bioinformatics, vol. 34, no. 15, pp. 2687-2689, 2018.
Candes & Tao, “Decoding by linear programming,” IEEE Trans. Inf. Theory, vol. 51, no. 12, pp. 4203-4215, December 2005.
Chen et al., “Atomic decomposition by basis pursuit,” SIAM J. Sci. Comput., vol. 20, no. 1, pp. 33-61, 1998.
Chen & Huo, “Sparse representations for multiple measurement vectors (MMV) in an over-complete dictionary,” in Proc. IEEE Int. Conf Acoust. Speech Signal Process., vol. 4, pp. 257-260, 2005.
Chen & Huo, “Theoretical results on sparse representations of multiplemeasurement vectors,” IEEE Trans. Signal Process., vol. 54, no. 12, pp. 4634-4643, December 2006.
Chen & Luss, “Stochastic gradient descent with biased but consistent gradient estimators,” doi.org/10.48550/arXiv.1807.11880, 2019.
Cleary et al., “Efficient generation of transcriptomic profiles by random composite measurements,” Cell Theory, vol. 171, no. 6, pp. 1424-1436.e18, November 2017.
Clemente et al., “The impact of the gut microbiota on human health: An integrative view,” Cell, vol. 148, no. 6, pp. 1258-1270, 2012.
Cohen et al., “Compressed sensing and best k-term approximation,” J. Am. Math. Soc., vol. 22, no. 1, pp. 211-231, January 2009.
Dalton et al., “An in vivo polymicrobial biofilm wound infection model to study interspecies interactions,” PLoS One, vol. 6, no. 11, 2011.
Dai & Milenkovic, “Weighted superimposed codes and constrained integer compressed sensing,” IEEE Trans. Inf. Theory, vol. 55, no. 5, pp. 2215-2229, May 2009a.
Dai & Milenkovic, “Subspace pursuit for compressive sensing signal reconstruction,” IEEE Trans. Inf. Theory, vol. 55, no. 5, pp. 2230-2249, May 2009b.
Davies & Eldar, “Rank awareness in joint sparse recovery,” IEEE Trans. Inf. Theory, vol. 58, no. 2, pp. 1135-1146, 2012.
David et al., “Diet rapidly and reproducibly alters the human gut microbiome,” Nature, vol. 505, no. 7484, pp. 559-563, 2014.
Dempster et al., “Maximum likelihood from incomplete data via the EM algorithm,” J. R. Stat. Soc. Series B, vol. 39, no. 1, pp. 1-38, 1977.
Dina et al., “Rapid singlecell detection and identification of pathogens by using surface-enhanced Raman spectroscopy,” Analyst, vol. 142, no. 10, pp. 1782-1789, 2017.
Donoho, “Compressed sensing,” IEEE Transactions on Information Theory, vol. 52, no. 4, pp. 1289-1306, 2006.
Draper & Malekpour, “Compressed sensing over finite fields,” in IEEE Int. Symp. Inf. Theory, pp. 669-673, June 2009.
Duarte et al., “Single-pixel imaging via compressive sampling,” IEEE Signal Process. Mag., vol. 25, no. 2, pp. 83-91, March 2008.
Ecker et al., “Ibis T5000: A universal biosensor approach for microbiology,” Nature Reviews Microbiology, vol. 6, no. 7, pp. 553-558, 2008.
Eldar & Kutyniok, Compressed Sensing: Theory and Applications, 1st ed. Cambridge University Press, May 2012.
Flores-Mireles et al., “Urinary tract infections: Epidemiology, mechanisms of infection and treatment options,” Nat. Rev. Microbiol., vol. 13, no. 5, pp. 269-284, 2015.
Flinth & Kutyniok, “PROMP: A sparse recovery approach to lattice-valued signals,” Appl. Comput. Harmon. Anal., vol. 45, pp. 668-708, March 2017.
Fukshansky et al., “An algebraic perspective on integer sparse recovery,” Appl. Math. Comput., vol. 340, no. 1, pp. 31-42, January 2019.
Galloway-Pena et al., “Characterization of oral and gut microbiome temporal variability in hospitalized cancer patients,” Genome Med., vol. 9, no. 1, pp. 1-14, 2017.
Gardy & Loman, “Towards a genomics-informed, real-time, global pathogen surveillance system,” Nature Reviews Genetics, vol. 19, no. 1, pp. 9-20, 2018.
Ghosh et al., “A compressed sensing approach to group-testing for covid-19 detection,” May 2020.
Grant & Boyd, “CVX: Matlab software for disciplined convex programming, version 2.1,” cvxr.com/cvx, March 2014.
Grant & Boyd, “Graph implementations for nonsmooth convex programs,” in Recent Advances in Learning and Control, ser. Lecture Notes in Control and Information Sciences, V. Blondel, S. Boyd, and H. Kimura, Eds. Springer-Verlag Limited, pp. 95-110, 2008, stanford.edu/˜boyd/graph dcp.html.
Guo et al., “Droplet microfluidics for high-throughput biological assays,” Lab Chip, vol. 12, pp. 2146-2155, February 2012.
Hall et al., “Human genetic variation and the gut microbiome in disease,” Nat. Rev. Genet., vol. 18, no. 11, pp. 690-699, 2017.
Harmany et al., “This is SPIRALTAP: Sparse poisson intensity reconstruction ALgorithms theory and practice,” IEEE Trans. Image Process., vol. 21, no. 3, pp. 1084-1096, March 2012.
Hosokawa et al., “Massively parallel whole genome amplification for single-cell sequencing using droplet microfluidics,” Sci. Rep., vol. 7, no. 5199, July 2017.
Ji et al., “Compressed sensing based multi-user detection with modified sphere detection in machine-tomachine communications,” in SCC 2015; 10th International ITG Conference on Systems, Communications and Coding, pp. 1-6, 2015.
Johnson et al., “Evaluation of 16S rRNA gene sequencing for species and strain-level microbiome analysis,” Nature Communications, vol. 10, no. 1, pp. 1-11, 2019.
Jones et al., “Low copy target detection by Droplet Digital PCR through application of a novel open access bioinformatic pipeline, ‘definetherain’,” Journal of Virological Methods, vol. 202, pp. 46-53, 2014.
Kim et al., “Large-scale femtoliter droplet array for digital counting of single biomolecules,” Lab Chip, vol. 12, no. 23, pp. 4986-4991, December 2012.
Kim & Haldar, “Greedy algorithms for nonnegativity constrained simultaneous sparse recovery,” Signal Processing, vol. 125, pp. 274-289, August 2016.
Koochakzadeh et al., “On fundamental limits of joint sparse support recovery using certain correlation priors,” IEEE Trans. Signal Process., vol. 66, no. 17, pp. 4612-4625, 2018.
Koslicki et al., “WGSQuikr: Fast whole-genome shotgun metagenomic classification,” PLoS ONE, vol. 9, no. 3, p. e91784, March 2014.
Kota et al., “Extreme Compressed Sensing of Poisson Rates from Multiple Measurements,” pp. 1-21, 2021.
Kota et al., “Extreme Compressed Sensing of Poisson Rates from Multiple Measurements,” IEEE Transactions on Signal Processing, pp. 1-14, 2022.
Land & Doig, “An automatic method of solving discrete programming problems,” Econometrica, vol. 28, no. 3, pp. 497-520, July 1960.
Lange et al., “Sparse recovery with integrality constraints,” Discrete Appl. Math., vol. 283, pp. 346-366, September 2020.
Langouche et al., “Data-driven noise modeling of digital DNA melting analysis enables prediction of sequence discriminating power,” Bioinformatics, vol. 36, no. 22-23, pp. 5337-5343, 2020.
Lasham et al., “Accessing a new dimension in TP53 biology: Multiplex long amplicon digital per to specifically detect and quantitate individual TP53 transcripts,” Cancers, vol. 12, no. 3, 2020.
Li et al., “Compressed sensing signal and data acquisition in wireless sensor networks and internet of things,” IEEE Trans. Ind. Informat., vol. 9, no. 4, pp. 2177-2186, November 2013.
Liu et al., “Scalable compressive sensing-based multi-user detection scheme for internet-of-things applications,” in IEEE Workshop on Signal Processing Systems (SiPS), pp. 1-6, 2015.
Luka et al., “Microfluidics integrated biosensors: A leading technology towards lab-on-A-chip and sensing applications,” Sensors (Switzerland), vol. 15, no. 12, pp. 30011-30031, 2015.
Lustig et al., “Compressed sensing MRI,” IEEE Signal Process. Mag., vol. 25, no. 2, pp. 72-82, March 2008.
MacBeath, “Protein microarrays and proteomics,” Nat. Genet., vol. 32, no. 4, pp. 526-532, December 2002.
Majumdar et al., “Digital PCR modeling for maximal sensitivity, dynamic range and measurement precision,” PLoS ONE, vol. 10, no. 3, pp. 1-17, 2015.
Marscha et al., “Evaluation of 3 different rapid automated systems for diagnosis of urinary tract infections,” Diagn. Microbiol. Infect. Dis., vol. 72, no. 2, pp. 125-130, 2012.
Mazutis et al., “Single-cell analysis and sorting using droplet-based microfluidics,” Nat. Protoc., vol. 8, no. 5, pp. 870-891, April 2013.
Monsees et al., “Reliable activity detection for massive machine to machine communication via multiple measurement vector compressed sensing,” in IEEE Globecom Workshops (GC Wkshps), pp. 1057-1062, December 2014.
Moon et al., “Statistical modeling of single target cell encapsulation,” PLoS ONE, vol. 6, no. 7, p. e21580, July 2011.
Moragues-Solanas et al., “Rapid metagenomics for diagnosis of bloodstream and respiratory tract nosocomial infections: current status and future prospects,” Expert Review of Molecular Diagnostics, vol. 21, no. 4, pp. 371-380, 2021.
Nakarmi & Rahnavard, “BCS: Compressive sensing for binary sparse signals,” in IEEE Military Communications Conf., pp. 1-5, 2012.
Needell & Tropp, “CoSaMP: Iterative signal recovery from incomplete and inaccurate samples,” Appl. Comput. Harmon. Anal., vol. 26, no. 3, pp. 301-321, May 2009.
Opota et al., “Microbial diagnosis of bloodstream infection: Towards molecular diagnosis directly from blood,” Clinical Microbiology and Infection, vol. 21, no. 4, pp. 323-331, 2015.
Orth et al., “Super-multiplexed fluorescence microscopy via photostability contrast,” Biomed. Opt. Express, vol. 9, no. 7, pp. 2943-2954, July 2018.
Ozenci et al., “Demise of Polymerase Chain Reaction/Electrospray” Ionization-Mass Spectrometry as an Infectious Diseases Diagnostic Tool,” Clinical Infectious Diseases, vol. 66, no. 3, pp. 452-455, 2018.
Pal & Vaidyanathan, “Pushing the limits of sparse support recovery using correlation information,” IEEE Trans. Signal Process., vol. 63, no. 3, pp. 711-726, 2015.
Palka-Santini et al., “Large scale multiplex per improves pathogen detection by dna microarrays,” BMC Microbiol., vol. 9, no. 1, January 2009.
Pati et al., “Orthogonal matching pursuit: recursive function approximation with applications to wavelet decomposition,” in Proceedings of 27th Asilomar Conference on Signals, Systems and Computers, vol. 1, pp. 40-44, 1993.
Peters et al., “Polymicrobial Interactions: Impact on Pathogenesis and Human Disease,” Clinical Microbiology Reviews, vol. 25, no. 1, pp. 193-213, 2012.
Quan et al., “dPCR: A technology review,” Sensors (Switzerland), vol. 18, no. 4, 2018.
Ragheb et al., “A prototype hardware for random demodulation based compressive analog-to-digital conversion,” in 51st Midwest Symposium on Circuits and Systems, vol. 4, pp. 37-40, 2008.
Raginsky et al., “Compressed sensing performance bounds under poisson noise,” IEEE Trans. Signal Process., vol. 58, no. 8, pp. 3990-4002, August 2010.
Schlaberg, “Microbiome diagnostics,” Clinical Chemistry, vol. 66, no. 1, pp. 68-76, 2020.
Schlaberg et al., “Validation of metagenomic next-generation sequencing tests for universal pathogen detection,” Arch. Path. Lab., vol. 141, no. 6, pp. 776-786, June 2017.
Shim et al., “Sparse detection with integer constraint using multipath matching pursuit,” IEEE Commun. Lett., vol. 18, no. 10, pp. 1851-1854, October 2014.
Sparrer & Fischer, “MMSE-based version of OMP for recovery of discrete-valued sparse signals,” Electron. Lett., vol. 52, no. 1, pp. 75-77, January 2016.
Sinha et al., “Emerging technologies for molecular diagnosis of sepsis,” Clin. Microbiol. Rev., vol. 31, no. 2, April 2018.
Singhal et al., “MALDI-TOF mass spectrometry: An emerging technology for microbial identification and diagnosis,” Frontiers in Microbiology, vol. 6, no. August, pp. 1-16, 2015.
Suea-Ngam et al., “Droplet microfluidics: from proof-of-concept to real-world utility?” Chem. Commun., vol. 55, no. 67, pp. 9895-9903, July 2019.
Tallis, “The identifiability of mixtures of distributions,” J. Appl. Prob., vol. 6, no. 2, pp. 389-398, 1969.
Tian et al., “Detection of sparse signals under finitealphabet constraints,” in Proc. IEEE Int. Conf Acoust. Speech Signal Process., pp. 2349-2352, April 2009.
Tropp, “Algorithms for simultaneous sparse approximation. Part II: Convex relaxation,” Signal Processing, vol. 86, no. 3, pp. 589-602, March 2006.
Tropp et al., “Algorithms for simultaneous sparse approximation. Part I: Greedy pursuit,” Signal Processing, vol. 86, no. 3, pp. 572-588, March 2006.
Teicher, “Identifiability of finite mixtures,” Ann. Math. Statist., vol. 34, no. 4, pp. 1265-1269, December 1963.
Velez et al., “Massively parallel digital high resolution melt for rapid and absolutely quantitative sequence profiling,” Scientific Reports, vol. 7, no. February, pp. 1-14, 2017.
Vogelstein & Kinzler, “Digital PCR,” PNAS, vol. 96, no. 16, pp. 9236-9241, August 1999.
Whale et al., “Fundamentals of multiplexing with digital PCR,” Biomolecular Detection and Quantification, vol. 10, pp. 15-23, 2016.
Wellinghausen et al., “Diagnosis of bacteremia in whole-blood samples by use of a commercial universal 16S rRNA gene-based PCR and sequence analysis,” Journal of Clinical Microbiology, vol. 47, no. 9, pp. 2759-2765, 2009.
Whale et al., “The Digital MIQE Guidelines Update: Minimum Information for Publication of Quantitative Digital PCR Experiments for 2020,” Clinical Chemistry, vol. 66, no. 8, pp. 1012-1029, 2020.
Willett et al., “Compressed sensing for practical optical imaging systems: a tutorial,” Opt. Eng., vol. 50, no. 7, July 2011.
Wu et al., “Biofilms in Chronic Wounds: Pathogenesis and Diagnosis,” Trends Biotechnol., vol. 37, no. 5, pp. 505-517, 2019.
Woods et al., “Development and application of a polymicrobial, in vitro, wound biofilm model,” J. Appl. Microbiol., vol. 112, no. 5, pp. 998-1006, 2012.
Xing et al., “Distance Metric Learning, with Application to Clustering with Side-Information,” Advances in Neural Information Processing Systems, vol. 15, pp. 505-512, 2002.
Yakowitz & Spragins, “On the identifiability of finite mixtures,” Ann. Math. Statist., vol. 39, no. 1, pp. 209-214, 1968.
Yang & Wu, “A new sufficient condition for identifiability of countably infinite mixtures,” Metrika, vol. 77, pp. 377-387, May 2013.
Yelleswarapu et al., “Mobile platform for rapid sub-picogram-per-milliliter, multiplexed, digital droplet detection of proteins,” PNAS, vol. 116, no. 10, pp. 4489-4495, February 2019.
Zhang et al., “Optimizing the specificity of nucleic acid hybridization,” Nat. Chem., vol. 4, pp. 208-214, January 2012.
Zhang et al., “A ‘culture’ shift: Application of molecular techniques for diagnosing polymicrobial infections,” Biotechnology Advances, vol. 37, no. 3, pp. 476-490, 2019.
Zhu & Fang, “Analytical detection techniques for droplet microfluidics—a review,” Anal. Chim. Acta, vol. 787, no. 17, pp. 24-35, July 2013.
Zhu & Giannakis, “Exploiting sparse user activity in multiuser detection,” IEEE Trans. Commun., vol. 59, no. 2, pp. 454-465, February 2011.

What is claimed:

1. A broad-range sensing method for detecting multiple target analytes in a sample comprising:
(a) assigning fingerprints to the target analytes with sensors;
(b) splitting the sample into multiple subsamples;
(c) splitting each subsample into multiple partitions;
(d) performing asynchronous fingerprinting by contacting the partitions in each subsample with a subset of the sensors; and
(e) detecting the multiple target analytes through statistical estimation using a reference database of analyte fingerprints.

2. The method of claim 1, wherein the sensors are nonspecific sensors.

3. The method of claim 1, wherein statistical estimation comprises a modeled probability distribution for the measurements in each partition that are conditional on the target analyte quantities the sample and the subset of sensors applied in the partition, further comprising:
(a) a single parametrization of the target analyte quantities or concentrations that is shared across all subsamples;
(b) a joint probability distribution for the measurements from partitions across all subsamples; and
(c) an objective function, based on the modeled probability distribution and the observed measurements from partitions, wherein the solution or optimization results in an estimate of the target analyte quantities or concentrations.

4. The method of claim 3, wherein the quantity of each analyte in each partition is an integer and follows a Poisson distribution with mean given by its true concentration in the sample, wherein the objective function comprises maximum likelihood estimation under the modeled probability distribution and maximum likelihood estimation comprises applying a gradient ascent or descent algorithm.

5. The method of claim 4, wherein the gradient ascent or descent algorithm is Sparse Poisson Recovery (SPoRe) algorithm comprising:
(a) initializing a value of λ;
(b) computing the gradient based on the modeled probability distribution over a subset of the partition measurements or over the entire set of available measurements, and optionally approximating the gradients with Monte Carlo approximations;
(c) updating λ based on the gradient; and
(d) repeating steps (b)-(c) until convergence of λ.

6. The method of claim 1, wherein the nonspecific sensors are nucleic acids, primers, or probes.

7. The method of claim 1, wherein detecting comprises quantifying the microbial content of the sample.

8. The method of claim 1, wherein the microfluidic partitions comprise droplets, chambers or nanowells.

9. The method of claim 1, wherein the signal describing the analyte quantities in the sample is sparse.

10. The method of claim 1, wherein the method comprises fewer total sensors than the number of target analytes.

11. The method of claim 1, wherein the target analytes are captured in the small volume partitions according to a Poisson distribution with the true Poisson rates of all target analytes totaling to less than 20.

12. The method of claim 1, wherein the subsamples are split into a total of 50 to 10⁷ partitions.

13. The method of claim 1, wherein the target analytes comprise whole cells, genomes, genes, DNA, RNA, or taxonomic groups.

14. The method of claim 1, wherein the target analytes comprise microbes, microbial genes, or mutations of interest.

15. The method of claim 1, wherein the target analytes comprise ribosomal RNA genes or gene regions selected from 16S, 18S, 23S, or 28S ribosomal RNA genes or other marker regions such as internal transcribed spacer (ITS) and interspace (IS) regions.

16. The method of claim 1, wherein the nonspecific sensors are nonspecific hydrolysis probes which react with the ribosomal RNA genes

17. The method of claim 1, wherein the nonspecific hydrolysis probes comprise 8-11 nucleotides with some bases substituted with locked nucleic acids and bind to the ribosomal RNA genes.

18. The method of claim 1, wherein the method comprises splitting a sample into microfluidic partitions in a digital PCR (dPCR) system and reading signatures from each partition at each PCR cycle or after performing PCR to detect target nucleic acids.

19. The method of claim 1, wherein the target analytes comprise microbes associated with urinary tract infection, bacterial biofilms in chronic wounds, sepsis, meningitis, or a human microbiome.

20. A method for evaluating a candidate solution for the target analyte quantities or concentrations in a sample that has been split into multiple partitions and reporting whether the sample contains an exogenous analyte beyond the set of target analytes comprising:
(d) a modeled probability distribution for the measurements in each partition that are conditional on the analyte quantities the sample and the subset of sensors applied in the partition;
(e) evaluating the expected distribution of measurements based on the modeled probability distribution and the candidate solution;
(f) comparing the expected distribution of measurements against the distribution of observed measurements obtained from the partitions, wherein a sufficient difference in the expected and observed distributions identifies the candidate solution as containing an exogenous analyte.


SEQUENCE LISTING (7 sequences)

SEQ ID NO: 1 - DNA, 20 bp, synthetic construct
  source 1..20: mol_type = other DNA
agagtttgatcmtggctcag

SEQ ID NO: 2 - DNA, 22 bp, synthetic construct
  source 1..22: mol_type = other DNA
tacggytaccttgttaygactt

SEQ ID NO: 3 - DNA, 11 bp, synthetic construct
  source 1..11: mol_type = other DNA
taacggctcac

SEQ ID NO: 4 - DNA, 11 bp, synthetic construct
  source 1..11: mol_type = other DNA
ctttcgcccat

SEQ ID NO: 5 - DNA, 11 bp, synthetic construct
  source 1..11: mol_type = other DNA
attccgacttc

SEQ ID NO: 6 - DNA, 11 bp, synthetic construct
  source 1..11: mol_type = other DNA
accaatccatc

SEQ ID NO: 7 - DNA, 11 bp, synthetic construct
  source 1..11: mol_type = other DNA
aagcactccgc